(19) United States
(12) Patent Application Publication
(10) Pub. No.: US 2008/0138411 A1
(43) Pub. Date: Jun. 12, 2008

(54) Modified Release Formulations Of Selective Serotonin Re-Uptake Inhibitors

(75) Inventors: Paul Jose Maes, Cuire (FR); Goutam Muhuri, Mississauga (CA)

Correspondence Address: OBLON, SPIVAK, MCCLELLAND MAIER & NEUSTADT, P.C., 1940 DUKE STREET, ALEXANDRIA, VA, 22314, US

(73) Assignee: BIOVAIL LABORATORIES INTERNATIONAL S.R.L, ST. MICHAEL (BB)

(21) Appl. No.: 10/556,492

(22) PCT Filed: Dec. 19, 2003
(86) PCT No.: PCT/CA03/01986

§ 371 Date: Jun. 6, 2007

(30) Foreign Application Priority Data
Dec. 24, 2002 (CA) 2415154

(51) Int. Cl. A61K 9/28; A61K 9/00; A61K 31/4525; A61K 31/343; A61K 31/138

(52) U.S. Cl. 424/468; 424/484; 514/321; 514/469; 514/652

(57) ABSTRACT

The present invention relates to modified release pharmaceutical compositions for oral administration and more particularly to modified release pharmaceutical compositions of a form of at least one selective serotonin re-uptake inhibitor selected from the group consisting of a selective serotonin reuptake inhibitor, racemic mixtures thereof, enantiomers thereof, pharmaceutically-acceptable salts thereof and combinations thereof.

### BACKGROUND OF THE INVENTION

Clinical depression (unipolar depression) is a disturbance of mood that is distinguishable from the usual mood fluctuations of everyday life. There are several approaches to the treatment of depression depending on the severity of the condition and the risks to the patient. Antidepressants are classified into different groups either structurally or depending on which central neurotransmitters they act upon. The older tricyclic and related cyclic antidepressants and the monoamine oxidase inhibitors (MAOIs) have now been joined by the highly selective neuronal erotonin (5HT) reuptake inhibitors (SSRIs) which provide important improvements in adverse effect profile and safety. The mechanism of action of the SSRIs as anti-depressants is presumed to be linked to potentiation of serotonergic activity in the central nervous system resulting from their inhibition of central nervous system (CNS) neuronal reuptake of serotonin. There are currently seven SSRIB available on the market today, namely Fluoxetine HCl, Fluvoxamine maleate, Paroxetine HCl, Sertraline HCl, Venlafaxine HCl, Citalopram HBr and Escitalopram oxalate.

Fluoxetine HCl, which was first described in U.S. Pat. No. 4,314,081, is designated as (±)-N-methyl-3-phenyl-3-[(α,α,α-trifluoro-p-tolyl)oxy)propylamine hydrochloride. Conventional immediate release preparations of Fluoxetine HCl are commercially available in the United States from Eli Lilly and Company under the proprietary name PROZAC® as 10 mg, 20 mg or 40 mg PULVULES®, 10 mg tablets and a 20 mg/5 ml oral solution. Each PULVULE® contains starch, gelatin, silicone, titanium dioxide, iron oxide and other inactive ingredients. The 10 mg and 20 mg PULVULES® also contain FD&C Blue No. 1, and the 40 mg PULVULE® also contains FD&C Blue No. 1 and FD&C Yellow No. 6. Each tablet contains microcrystalline cellulose, magnesium stearate, crospovidone, hydroxypropyl methylcellulose, titanium dioxide, polyethylene glycol, yellow iron oxide, FD&C Blue No. 1 aluminum lake, and polysorbate 80. The oral solution contains alcohol 0.23%, benzoic acid, flavoring agent, glycerin, purified water, and sucrose. The 56^{th} Edition (2002) of the Physician's Desk Reference (PDR), page 1238, states that following a single oral 40 mg dose, peak plasma concentrations of Fluoxetine from 15 to 55 mg/ml are observed after 6 to 8 hours. The PULVULE®, tablet and oral solution dosage forms of Fluoxetine are bioequivalent. Food does not appear to affect the systemic bioavailability of Fluoxetine, although it may delay its absorption by one to two hours, which is probably not clinically significant. Thus, Fluoxetine may be administered with or without food. Such conventional immediate release preparations, however, do not provide a modified release of Fluoxetine HCl.

A delayed release preparation of Fluoxetine HCl is also commercially available in the United States from Ely Lilly and Company under the proprietary name PROZAC® WEEKLY™ as 90 mg delayed release capsules, containing enteric coated pellets of Fluoxetine HCl. The capsules also contain FD&C Yellow #10, FD&C Blue #2, gelatin, hydroxypropyl methylcellulose, hydroxypropyl methylcellulose acetate succinate, sodium lauryl sulfate, sucrose, sugar spheres, talc, titanium dioxide, triethylcitrate and other inactive ingredients. PROZAC® WEEKLY™ capsules contain enteric coated pellets that resist dissolution until reaching a segment of the gastrointestinal tract where the pH exceeds 5.5. The enteric coating delays the onset of absorption of Fluoxetine one to two hours relative to the immediate release formulations. The PROZAC® WEEKLY™ delayed release capsule dosage form of Fluoxetine is bioequivalent to the PULVULE®, tablet and oral solution dosage forms of Fluoxetine. The PROZAC® WEEKLY™ delayed release capsule formulation is disclosed in U.S. Pat. No. 5,910,319.

Fluvoxamine maleate, which was first described in U.S. Pat. No. 4,085,225, is an SSRI belonging to a new chemical series, the 2-aminoethyl oxime ethers of aralkylketones and is designated as 5-methoxy-4′-(trifluoromethyl)valerophenone-(E)-O-(2-aminoethyl)oxime maleate. A conventional immediate release preparation of Fluvoxamine maleate is commercially available in the United States from Solvay Pharmaceuticals, Inc. under the proprietary name LUVOX® as 25 mg, 50 mg and 100 mg tablets for oral administration. Each tablet contains the following inactive ingredients: carnauba wax, hydroxypropyl methylcellulose, mannitol, polyethylene glycol, polysorbate 80, pregelatinized starch (potato), silicon dioxide, sodium stearyl fumarate, starch (corn), and titanium dioxide. The 50 mg and 100 mg tablets also contain synthetic iron oxides. The 56^{th} Edition of the PDR, page 3257, states that the absolute bioavailability of Fluvoxamine maleate is 53%. Oral bioavailability is not significantly affected by food. In a dose proportionality study involving Fluvoxamine maleate at 100, 200 and 300 mg/day for 10 consecutive days in 30 normal volunteers, steady state was achieved after about a week of dosing. Maximum plasma concentrations at steady state occurred within 38 hours of dosing and reached concentrations averaging 88, 283 and 546 ng/ml, respectively. Such a conventional immediate release preparation, however, does not provide a modified release of Fluvoxamine maleate.

Paroxetine HCl, which was first described in U.S. Pat. Nos. 3,912,743, 4,007,196 and 4,721,723, is an orally administered antidepressant in the hemihydrate form with a chemical structure unrelated to other SSRIs or to tricyclic, tetracyclic or other available antidepressant agents. It is the hydrochloride salt of a phenylpiperidine compound identified chemically as (−)-trans-4R-(4′-fluorophenyl)-3S[(3′,4′-methylenedioxyphenoxy)methyl]piperidine hydrochloride (hemihydrate). Conventional immediate release preparations of Paroxetine HCl are commercially available in the United States from GlaxoSmithKline under the proprietary name PAXIL® as 10 mg, 20 mg, 30 mg and 40 mg film coated tablets and as a 10 mg/5 ml suspension for oral administration. Each film coated tablet contains the inactive ingredients dibasic calcium phosphate dihydrate, hydroxypropyl methylcellulose, magnesium stearate, polyethylene glycols, polysorbate 80, sodium starch glycolate, titanium dioxide and one or more of the following: D&C Red No. 30, D&C Yellow No. 10, FD&C Blue No. 2 and FD&C Yellow No. 6. The oral suspension contains the inactive ingredients polacrilin potassium, microcrystalline cellulose, propylene glycol, glycerin, sorbitol, methyl paraben, propyl paraben, sodium citrate dihydrate, citric acid anhydrate, sodium saccharin, flavourings, FD&C Yellow No. 6 and simethicone emulsion, USP. The 56^{th} Edition of the PDR, page 1609 states that in a study in which normal male subjects received 30 mg tablets daily for 30 days, steady state Paroxetine concentrations were achieved by approximately 10 days for most subjects. At steady state, mean values of Cₘₐₓ, Tₘₐₓ, Cₘᵢₙ and t_{1/2} were 61.7 ng/ml (CV 45%), 5.2 hours (CV 10%), 30.7 ng/ml (CV 67%) and 21.0 hours (CV 32%), respectively. The steady state Cₘₐₓ and Cₘᵢₙ values were about 6 and 14 times what would be predicted from single dose studies. Steady state drug exposure based on AUC₀₋₂₄ was about 8 times greater than would have been predicted from single dose data in these subjects. The effects of food on the bioavailability of Paroxetine were studied and subjects were administered a single dose with and without food. AUC was only slightly increased (6%) when drug was administered with food, but the Cₘₐₓ 29% greater, while the time to reach peak plasma concentration decreased from 6.4 hours post dosing to 4.9 hours. Such conventional immediate release preparations, however, do not provide a modified release of Paroxetine HCl.

A controlled release preparation of Paroxetine HCl is also commercially available in the United States from GlaxoSmithKline under the proprietary name PAXIL® CR™ as 12.5 mg, 25 mg and 37.5 mg enteric film coated, controlled release tablets. One layer of the tablet consists of a degradable barrier layer and the other contains the active material in a hydrophilic matrix. Inactive ingredients consist of hydroxypropyl, methylcellulose, polyvinyl pyrolidone, lactose monohydrate, magnesium stearate, colloidal silicon dioxide, glyceryl behenate, methacrylic acid copolymer type C, sodium lauryl sulfate, polysorbate 80, talc, triethyl citrate, and one or more of the following colorants: yellow ferric oxide, red ferric oxide, D&C Red #30, D&C Yellow #6, D&C Yellow #10, FD&C Blue #2. PAXIL® CR™ tablets contain a degradable polymer matrix (GEOMATRIX™, a trademark of Jago Pharma Muttenz, Switzerland) designed to control the dissolution rate of Paroxetine over a period of approximately 4 to 5 hours. In addition to controlling the rate of drug release in vivo, an enteric coat delays the start of drug release until PAXIL® CR™ tablets have left the stomach. In a study in which normal male and female subjects (n=23) received single oral doses of PAXIL® CR™ at 4 dosage strengths (12.5 mg, 25 mg, 37.5 mg and 50 mg), Paroxetine mean Cₘₐₓ and AUC_{0-inf} values at these doses were 2.0, 5.5, 9.0 and 12.5 mg/ml and 121, 261, 338 and 540 ng.hr./ml, respectively. Tₘₐₓ was observed typically between 6 and 10 hours post dose, reflecting a reduction in absorption rate compared with immediate release formulations. The mean elimination half life of Paroxetine was 15 to 20 hours throughout this range of single PAXIL® CR™ doses. The bioavailability of 25 mg PAXIL® CR™ is not affected by food. During repeated administration of PAXIL® CR™ (25 mg once daily), steady state was reached within two weeks (i.e., comparable to immediate release formulations). In a repeat dose study in, which normal male and female subjects (n=23) received PAXIL® CR™ (25 mg daily), mean steady state Cₘₐₓ, Cₘᵢₙ and AUC₀₋₂₄ values were 30 ng/ml, 20 ng/ml and 550 ng.hr./ml, respectively. According to the United States Food and Drug Administration (FDA) Approved Drug Products Publication (the “FDA Orange Book”), U.S. Pat. No. 4,522,123 relates to the PAXIL® CR™ controlled release tablet formulation.

Sertraline HCl, which was first described in U.S. Pat. No. 4,536,518, is chemically unrelated to other SSRIs, tricyclic, tetracyclic, or other available antidepressant agents. Sertraline HCl has the following chemical name: (1S-cis)-4(3,4-dichlorophenyl)-1,2,3,4-tetrahydro-N-methyl-1-naphthalenamine hydrochloride. Conventional immediate release preparations of Sertraline HCl are commercially available in the United States from Pfizer Inc. under the trade name ZOLOFT® as 25 mg, 50 mg and 100 mg scored tablets and as 20 mg/ml oral concentrate. The tablets contain the following inactive ingredients: dibasic calcium phosphate dihydrate, D&C Yellow #10 aluminum lake (in 25 mg tablet), FD&C Blue #1 aluminum lake (in 25 mg tablet), FD&C Red #40 aluminum lake (in 25 mg tablet), FD&C Blue #2 aluminum lake (in 50 mg tablet), hydroxypropyl cellulose, hydroxypropyl methylcellulose, magnesium stearate, microcrystalline cellulose, polyethylene glycol, polysorbate 80, sodium starch glycolate, synthetic yellow iron oxide (in 100 mg tablet), and titanium dioxide. The solution contains the following inactive ingredients: glycerine, alcohol (12%), menthol and butylated hydroxytoluene (BHT). The 56^{th} Edition of the PDR, on page 2751, states that in man, following oral once daily dosing over the range of 50-200 mg for 14 days, mean peak plasma concentrations (Cₘₐₓ) of Sertraline occurred between 4.5-8.4 hours post dosing. The affects of food on the bioavailability of the Sertraline tablet and oral concentrate were studied in subjects administered a single dose with and without food. For the tablet, area under the plasma concentration time curve (AUC) was slightly increased when drug was administered with food but the Cₘₐₓ was 25% greater, while the time to reach peak plasma concentration (Tₘₐₓ) decreased from 8 hours post dosing to 5.5 hours. For the oral concentrate, Tₘₐₓ was slightly prolonged from 5.9 hours to 7.0 hours with food. Such conventional immediate release preparations, however, do not provide a modified release of Sertraline HCl.

Venlafaxine HCl, which was first described in U.S. Pat. No. 4,535,186, is chemically unrelated to tricyclic, tetracyclic, or other available antidepressant agents. It is designated as (R/S)-1-[2-(dimethylamino)-1-(4-methoxyphenyl)ethyl]cyclohexanol hydrochloride or (±)-1-[α-[(dimethylamino)methyl]-p-methoxybenzyl]cyclohexanol hydrochloride. A conventional immediate release preparation of Venlafaxine HCl is commercially available in the United States from Wyeth-Ayerst under the proprietary name EFFEXOR® as 25 mg, 37.5 mg, 50 mg, 75 mg or 100 mg compressed tablets. Inactive ingredients consist of cellulose, iron oxides, lactose, magnesium stearate, and sodium starch glycolate. Such a conventional immediate release preparation does not provide a modified release of Venlafaxine HCl.

An extended release preparation of Venlafaxine HCl is also available from Wyeth-Ayerst under the proprietary name EFFEXOR® XR as 37.5 mg, 75 mg or 150 mg extended release capsules for once a day oral administration. Drug release is controlled by diffusion through the coating membrane on the spheroids and is not pH dependent. Inactive ingredients consist of cellulose, ethylcellulose, gelatin, hydroxypropyl methylcellulose, iron oxide and titanium dioxide. The 37.5 mg capsule also contains D&C Red #28, D&C Yellow #10, and FD&C Blue #1. The 56^{th} Edition of the PDR, page 3499, states that administration of EFFEXOR® XR (150 mg q24 hours) generally resulted in lower Cₘₐₓ (150 ng/ml) and later Tₘₐₓ (5.5 hours) than for immediate release Venlafaxine tablets (Cₘₐₓ for immediate release 75 mg q12 hours was 225 ng/ml; Tₘₐₓ was 2 hours). The FDA Orange Book states that U.S. Pat. Nos. 6,274,171, 6,419,958 and 6,403,120 all relate to the EFFEXOR® XR extended release capsule formulation.

Citalopram HBr, which was first described in U.S. Pat. No. 4,136,193, is a racenic bicyclic phthalane derivative designated as (RS)-1-[3-(dimethylamino)propyl]-1-(4-fluorophenyl)-5-phthalancarbonitrile hydrobromide. Conventional immediate release preparations of Citalopram HBr are commercially available in the United States from Forest Laboratories, Inc. under the proprietary name CELEXA™ as 20 mg and 40 mg film coated tablets and as 2 mg/ml oral solution. The tablets contain the following inactive ingredients: copolyvidone, corn starch, cross-carmellose sodium, glycerin, lactose monohydrate, magnesium stearate, hydroxypropyl methylcellulose, microcrystalline cellulose, polyethylene glycol and titanium dioxide. Iron oxides are used as colouring agents in the 10 mg and 20 mg tablets. The oral solution contains the following inactive ingredients: sorbitol, purified water, propylene glycol methylparaben, natural peppermint flavour and propylparaben. The 56^{th} Edition of the PDR, page 1365, states that following a single oral dose (40 mg tablet) of Citalopram, peak blood levels occur at about 4 hours. The absolute bioavailability of Citalopram was about 80% relative to an intravenous dose and absorption is not affected by food. Such conventional immediate release preparations, however, do not provide a modified release of Citalopram HBr.

Escitalopram oxalate, which was first described in U.S. Pat. No. RE 34712, is the pure S-enantiomer (single isomer) of the racemic bicyclic phthalane derivative of Citalopram. Escitalopram oxalate is designated as S-(+)-1-[3-(dimethyl-amino)propyl]-1-(β-fluorophenyl)-5-phthalancarbonitrile oxalate. A conventional immediate release preparation of Escitalopram oxalate is commercially available in the United States from Forest Laboratories, Inc. under the proprietary name LEXAPRO™ as 10 mg and 20 mg film coated tablets. The tablets contain the following inactive ingredients: talc, croscarmellose sodium, microcrystalline cellulose/colloidal silicon dioxide and magnesium stearate. The film coating contains hydroxypropyl methylcellulose, titanium dioxide and polyethylene glycol. The single and multiple dose pharmacokinetics of Escitalopram are linear and dose proportional in a dose range of 10 to 30 mg per day. Biotransformation of Escitalopram is mainly hepatic with a mean terminal half life of about 27 to 32 hours. With once daily dosing, steady state plasma concentrations are achieved within approximately one week. At steady state, the extent of accumulation of Escitalopram in plasma in young healthy subjects was 2.2 to 2.5 times the plasma concentrations observed after a single dose. Following a single oral dose (20 mg tablet) of Escitalopram, the mean Tₘₐₓ was 5±1.5 hours. Absorption of Escitalopram is not affected by food. The FDA Orange Book states that U.S. Pat. No. RE 34712 relates to the LEXAPRO™ formulation. Such a conventional immediate release preparation, however, does not provide a modified release of Escitalopram oxalate.

### SUMMARY OF THE INVENTION

In accordance with one aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof, and combinations thereof in combination with at least one pharmaceutically-acceptable excipient, wherein the pharmaceutical composition, when orally administered to a patient, induces a statistically significant lower mean fluctuation index in the plasma than an immediate release composition of the form of the at least one SSRI while maintaining bioavailability substantially equivalent to that of the immediate release composition.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof, and combinations thereof in combination with at least one pharmaceutically-acceptable excipient, wherein the pharmaceutical composition, when orally administered to a patient, produces a mean maximum plasma concentration (Cₘₐₓ) of the form of the at least one SSRI that is lower than that produced by an immediate release pharmaceutical composition of the form of the at least one SSRI, and the area under the plasma concentration-time curve (AUC) and the mean minimum plasma concentration (Cₘᵢₙ) are substantially equivalent to that of the immediate release pharmaceutical composition.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof, and combinations thereof in combination with at least one pharmaceutically-acceptable excipient, wherein the pharmaceutical composition, when orally administered to a patient, produces a mean maximum plasma concentration (Cₘₐₓ) of the form of the at least one SSRI and an area under a plasma concentration vs. time curve (AUC) within the range of from about −20% to about +25% of that produced by an immediate release pharmaceutical composition of the form of the at least one SSRI.

In an embodiment of the present invention, the form of the at least one SSRI is Citalopram HBr and the immediate release pharmaceutical composition is the subject of the United States Food and Drug Administration Approved New Drug Application number N20822 or N21046.

In an embodiment of the present invention, the form of the at least one SSRI is Escitalopram oxalate and the immediate release pharmaceutical composition is the subject of the United States Food and Drug Administration Approved New Drug Application number N21323.

In an embodiment of the present invention, the form of the at least one SSRI is Fluoxetine HCl and the immediate release pharmaceutical composition is the subject of the United States Food and Drug Administration ‘Approved New Drug Application’ number N18936, N20101, N20974, or N75755.

In an embodiment of the present invention, the form of the at least one SSRI is Fluvoxamine maleate and the immediate release pharmaceutical composition is the subject of the United States Food and Drug Administration Approved New Drug Application number N20243 or N75888.

In an embodiment of the present invention, the form of the at least one. SSRI is Sertraline HCl and the immediate release pharmaceutical composition is the subject of the United States Food and Drug Administration Approved New Drug Application number N20990 or N19839.

In an embodiment of the present invention, the form of the at least one SSRI is Verlafaxine HCl and the immediate release pharmaceutical composition is the subject of the United States Food and Drug Administration Approved New Drug Application number N20151.

In an embodiment of the present invention, the form of the at least one SSRI is Paroxetine HCl and the immediate release pharmaceutical composition is the subject of the United States Food and Drug Administration Approved New Drug Application number N20710 or N20031.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIS, racemic mixtures thereof, enantiomers thereof, pharmaceutically acceptable salts thereof, and combinations thereof in combination with at least one pharmaceutically-acceptable excipient wherein the pharmaceutical composition exhibits the following in vitro dissolution profile when measured using the USP Paddle Method at 100 rpm in 900 mL of a buffered medium having a pH between about 5.5 and about 7.5 at 37.0±0.5° C.:
(a) between about 0% and about 50% (by wt) of the form of the at least one SSRI is released after about 1 hour;
(b) between about 0% and about 75% (by wt) of the form of the at least one SSRI is released after about 2 hours;
(c) between about 3% and about 95% (by wt) of the form of the at least one SSRI is released after about 4 hours;
(d) between about 10% and about 100% (by wt) of the form of the at least one SSRI is released after about 8 hours;
(e) between about 20% and about 100% (by wt) of the form of the at least one SSRI is released after about 12 hours;
(f) between about 30% and about 100% (by wt) of the form of the at least one SSRI is released after about 16 hours;
(g) between about 50% and about 100% (by wt) of the form of the at least one SSRI is released after about 24 hours; and
(h) in excess of about 80% (by wt) of the form of the at least one SSRI is released after about 36 hours.

In an embodiment of the present invention, the composition exhibits the following in vitro dissolution profile when measured using the USP paddle method at 100 rpm in 900 ml of a buffered medium having a pH from about 5.5 to about 7.5 at 37.0±0.5° C.:
(a) from about 20% to about 50% (by weight) of the form of the at least one SSRI is released after about 1 hour;
(b) from about 40% to about 75% (by weight) of the form of the at least one SSRI is released after about 2 hours;
(c) from about 60% to about 95% (by weight) of the form of the at least one SSRI is released after about 4 hours;
(d) from about 80% to about 100% (by weight) of the form of the at least one SSRI is released after about 8 hours; and
(e) from about 90% to about 100% (by weight) of the form of the at least one SSRI is released after about 12 hours.

In an embodiment of the present invention, the composition exhibits the following in vitro dissolution profile when measured using the USP paddle method at 100 rpm in 900 ml of a buffered medium having a pH from about 5.5 to about 7.5 at 37.0±0.5° C.:
(a) from about 0% to about 50% (by weight) of the form of the at least one SSRI is released after about 1 hour;
(b) from about 0% to about 75% (by weight) of the form of the at least one SSRI is released after about 2 hours;
(c) from about 10% to about 95% (by weight) of the form of the at least one SSRI is released after about 4 hours;
(d) from about 35% to about 100% (by weight) of the form of the at least one SSRI is released after about 8 hours;
(e) from about 55% to about 100% (by weight) of the form of the at least one SSRI is released after about 12 hours;
(f) from about 70% to about 100% (by weight) of the form of the at least one SSRI is released after about 16 hours; and
(g) in excess of about 90% (by weight) of the form of the at least one SSRI is released after about 24 hours.

In an embodiment of the present invention, the composition exhibits the following in vitro dissolution profile when measured using the USP paddle method at 100 rpm in 900 ml of a buffered medium having a pH from about 5.5 to about 7.5 at 37.0±0.5° C.:
(a) from about 0% to about 30% (by weight) of the form of the at least one SSRI is released after about 1 hour;
(b) from about 0% to about 45% (by weight) of the form of the at least one SSRI is released after about 2 hours;
(c) from about 3% to about 55% (by weight) of the form of the at least one SSRI is released after about 4 hours;
(d) from about 10% to about 65% (by weight) of the form of the at least one SSRI is released after about 8 hours;
(e) from about 20% to about 75% (by weight) of the form of the at least one SSRI is released after about 12 hours;
(f) from about 30% to about 88% (by weight) of the form of the at least one SSRI is released after about 16 hours;
(g) from about 50% to about 100% (by weight) of the form of the at least one SSRI is released after about 24 hours; and
(h) in excess of about 80% (by weight) of the form of the at least one SSRI is released after about 36 hours.

In an embodiment of the present invention, the composition exhibits the following in vitro dissolution profile when measured using the USP paddle method at 100 rpm in 900 ml of a buffered medium having a pH from about 5.5 to about 7.5 at 37.0±0.5° C.:
(a) from about 5% to about 50% (by weight) of the form of the at least one SSRI is released after about 1 hour;
(b) from about 10% to about 75% (by weight) of the form of the at least one SSRI is released after about 2 hours;
(c) from about 20% to about 95% (by weight) of the form of the at least one SSRI is released after about 4 hours;
(d) from about 40% to about 100% (by weight) of the form of the at least one SSRI is released after about 8 hours;
(e) more than about 50% (by weight) of the form of the at least one SSRI is released after about 12 hours;
(f) more than about 70% (by weight) of the form of the at least one SSRI is released after about 18 hours; and
(g) more than about 80% (by weight) of the form of the at least one SSRI is released after about 24 hours.

In an embodiment of the present invention, the composition exhibits the following in vitro dissolution profile when measured using the USP paddle method at 100 rpm in 900 ml of a buffered medium having a pH from about 5.5 to about 7.5 at 37.0±0.5° C.:
(a) from about 15% to about 25% (by weight) of the form of the at least one SSRI is released after about 1 hour;
(b) from about 25% to about 35% (by weight) of the form of the at least one SSRI is released after about 2 hours;
(c) from about 30% to about 45% (by weight) of the form of the at least one SSRI is released after about 4 hours;
(d) from about 40% to about 60% (by weight) of the form of the at least one SSRI is released after about 8 hours;
(e) from about 55% to about 70% (by weight) of the form of the at least one SSRI is released after about 12 hours; and
(f) from about 60% to about 75% (by weight) of the form of the at least one SSRI is released after about 16 hours.

In an embodiment of the present invention, the composition exhibits the following in vitro dissolution profile when measured using the USP paddle method at 100 rpm in 900 ml of a buffered medium having a pH from about 5.5 to about 7.5 at 37.0±0.5° C.:
(a) from about 10% to about 30% (by weight) of the form of the at least one SSRI is released after about 1 hour;
(b) from about 46% to about 66% (by weight) of the form of the at least one SSRI is released after about 4 hours;
(c) from about 70% to about 90% (by weight) of the form of the at least one SSRI is released after about 8 hours; and
(d) in excess of about 80% (by weight) of the form of the at least one SSRI is released after about 12 hours.

In an embodiment of the present invention, the pharmaceutical composition exhibits the following in vitro dissolution profile when measured using the USP Paddle Method at 100 rpm in 900 ml of a buffered medium, having a pH between about 5.5 and about 7.5 at 37.0±0.5° C.:
(a) from about 3% to about 20% (by weight) of the form of the at least one SSRI is release after about 1 hour;
(b) from about 7% to about 32% (by weight) of the form of the at least one SSRI is released after about 2 hours;
(c) from about 17% to about 50% (by weight) of the form of the at least one SSRI is released after about 4 hours;
(d) from about 30% to about 75% (by weight) of the form of the at least one SSRI is released after about 8 hours;
(e) from about 55% to about 90% (by weight) of the form of the at least one SSRI is released after about 12 hours;
(f) from about 68% to about 98% (by weight) of the form of the at least one SSRI is released after about 16 hours; and
(g) in excess of about 85% (by weight) of the form of the at least one SSRI is released after about 24 hours.

In an embodiment of the present invention, the pharmaceutical composition exhibits the following in vitro dissolution profile when measured using the USP Paddle Method at 100 rpm in 900 ml of a buffered medium, having a pH between about 5.5 and about 7.5 at 37.0±0.5° C.:
(a) from about 18% to about 30% (by weight) of the form of the at least one SSRI is release after about 1 hour;
(b) from about 28% to about 43% (by weight) of the form of the at least one SSRI is released after about 2 hours;
(c) from about 45% to about 78% (by weight) of the form of the at least one SSRI is released after about 4 hours;
(d) from about 68% to about 98% (by weight) of the form of the at least one SSRI is released after about 8 hours;
(e) from about 82% to about 100% (by weight) of the form of the at least one SSRI is released after about 12 hours;
(f) from about 90% to about 100% (by weight) of the form of the at least one SSRI is released after about 16 hours; and
(g) in excess of about 95% (by weight) of the form of the at least one SSRI is released after about 24 hours.

In an embodiment of the present invention, the pharmaceutical composition exhibits the following in vitro dissolution profile when measured using the USP Paddle Method at 100 rpm in 900 ml of a buffered medium, having a pH from about 5.5 and about 7.5 at 37.0±0.5° C.:
(a) from about 10% to about 22% (by weight) of the form of the at least one SSRI is release after about 1 hour;
(b) from about 16% to about 35% (by weight) of the form of the at least one SSRI is released after about 2 hours;
(c) from about 28% to about 55% (by weight) of the form of the at least one SSRI is released after about 4 hours;
(d) from about 48% to about 80% (by weight) of the form of the at least one SSRI is released after about 8 hours;
(e) from about 62% to about 100% (by weight) of the form of the at least one SSRI is released after about 12 hours;
(f) from about 73% to about 100% (by weight) of the form of the at least one SSRI is released after about 16 hours; and
(g) in excess of about 80% (by weight) of the form of the at least one SSRI is released after about 24 hours.

In an embodiment of the present invention, the pharmaceutical composition exhibits the following in vitro dissolution profile when measured using the USP Paddle Method at 100 rpm in 900 ml of a buffered medium, having a pH from about 5.5 and about 7.5 at 37.0±0.5° C.:
(a) from about 20% to about 24% (by weight) of the form of the at least one SSRI is release after about 1 hour;
(b) from about 30% to about 38% (by weight) of the form of the at least one SSRI is released after about 2 hours;
(c) from about 50% to about 58% (by weight) of the form of the at least one SSRI is released after about 4 hours;
(d) from about 75% to about 85% (by weight) of the form of the at least one SSRI is released after about 8 hours;
(e) from about 90% to about 100% (by weight) of the form of the at least one SSRI is released after about 12 hours; and
(f) in excess of about 90% (by weight) of the form of the at least one SSRI is released after about 16 hours.

In an embodiment of the present invention, the pharmaceutical composition exhibits the following in vitro dissolution profile when measured using the USP Paddle Method at 100 rpm in 900 ml of a buffered medium, having a pH from about 5.5 and about 7.5 at 37.0±0.5° C.:
(a) from about 20% to about 25% (by weight) of the form of the at least one SSRI is release after about 1-hour;
(b) from about 32% to about 38% (by weight) of the form of the at least one SSRI is released after about 2 hours;
(c) from about 50% to about 60% (by weight) of the form of the at least one SSRI is released after about 4 hours;
(d) from about 75% to about 85% (by weight) of the form of the at least one SSRI is released after about 8 hours;
(e) from about 90% to about 100% (by weight) of the form of the at least one SSRI is released after about 12 hours; and
(f) in excess of about 90% (by weight) of the form of the at least one SSRI is released after about 16 hours.

In accordance with another aspect of the present invention, there is provided a modified release pharmaceutical composition for oral administration, suitable for once daily dosing, comprising about 20 mg of a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition when orally administered to a patient, provides a mean maximum plasma concentration (Cₘₐₓ) of the form of the at least one SSRI from about 5.0 ng/ml to about 28.0 ng/ml.

In an embodiment of the present invention, the composition when orally administered to a fasting patient, provides a Cₘₐₓ of the form of the at least one SSRI from about 5.0 ng/ml to about 28.0 ng/ml.

In an embodiment of the present invention, the composition when orally administered to a fasting patient, provides a Cₘₐₓ of the form of the at least one SSRI from about 7.0 ng/ml to about 14.0 ng/ml.

In an embodiment of the present invention, the composition when orally administered to a fasting patient, provides a Cₘₐₓ of the form of the at least one SSRI from about 8.0 ng/ml to about 12.0 ng/ml.

In an embodiment of the present invention, the composition when orally administered to a fasting patient, provides a Cₘₐₓ of the form of the at least one SSRI from about 8.0 ng/ml to about 15.0 ng/ml.

In an embodiment of the present invention, the composition when orally administered to a fasting patient, provides a Cₘₐₓ of the form of the at least one SSRI from about 10.0 ng/ml to about 14.0 ng/ml.

In an embodiment of the present invention, the composition when orally administered to a fasting patient, provides a C of the form of the at least one SSRI from about 13.0 ng/ml to about 21.0 ng/ml.

In an embodiment of the present invention, the composition when orally administered to a fasting patient, provides a Cₘₐₓ of the form of the at least one SSRI from about 15.0 ng/ml to about 19.0 ng/ml.

In an embodiment of the present invention, the composition when orally administered to a fasting patient, provides a Cₘₐₓ of the form of the at least one SSRI from about 5.0 ng/ml to about 14.0 ng/ml.

In an embodiment of the present invention, the composition when orally administered to a fasting patient, provides a Cₘₐₓ of the form of the at least one SSRI from about 9.0 ng/ml to about 13.0 ng/ml.

In an embodiment of the present invention, the composition when orally administered to a fasting patient, provides a Cₘₐₓ of the form of the at least one SSRI from about 5.0 ng/ml to about 16.0 ng/ml.

In an embodiment of the present invention, the composition when orally administered to a fasting patient, provides a Cₘₐₓ of the form of the at least one SSRI from about 12.0 ng/ml to about 19.0 ng/ml.

In an embodiment of the present invention, the composition when orally administered to a fasting patient, provides a Cₘₐₓ of the form of the at least one SSRI from about 13.0 ng/ml to about 17.0 ng/ml.

In an embodiment of the present invention, the composition when orally administered to a fasting patient, provides a Cₘₐₓ of the form of the at least one SSRI from about 14.0 ng/ml to about 21.0 ng/ml.

In an embodiment of the present invention, the composition when orally administered to a fasting patient, provides a Cₘₐₓ of the form of the at least one SSRI from about 14.0 ng/ml to about 18.0 ng/ml.

In an embodiment of the present invention, the composition when orally administered to a fasting patient, provides a Cₘₐₓ of the form of the at least one SSRI from about 13.0 ng/ml to about 25.0 ng/ml.

In an embodiment of the present invention, the composition when orally administered to a fasting patient, provides a Cₘₐₓ of the form of the at least one SSRI from about 16.0 ng/ml to about 20.0 ng/ml.

In an embodiment of the present invention, the composition when orally administered to a fasting patient, provides a Cₘₐₓ of the form of the at least one SSRI from about 6.0 ng/ml to about 28.0 ng/ml.

In an embodiment of the present invention, the composition when orally administered to a fasting patient, provides a Cₘₐₓ of the form of the at least one SSRI from about 15.0 ng/ml to about 19.0 ng/ml.

In an embodiment of the present invention, the composition when orally administered to a fed patient, provides a Cₘₐₓ of the form of the at least one SSRI from about 9.0 ng/ml to about 18.0 ng/ml.

In an embodiment of the present invention, the composition when orally administered to a fed patient, provides a Cₘₐₓ of the form of the at least one SSRI from about 9.0 ng/ml to about 16.0 ng/ml.

In an embodiment of the present invention, the composition when orally administered to a fed patient, provides a Cₘₐₓ of the form of the at least one SSRI from about 11.0 ng/ml to about 15.0 ng/ml.

In an embodiment of the present invention, the composition when orally administered to a fed patient, provides a Cₘₐₓ of the form of the at least one SSRI from about 11.0 to about 18.0 ng/mL.

In an embodiment of the present invention, the composition when orally administered to a fed patient, provides a Cₘₐₓ of the form of the at least one SSRI from about 13.0 ng/ml to about 17.0 ng/ml.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof pharmaceutically acceptable salts thereof and combinations thereof in combination with at least one pharmaceutically-acceptable excipient, wherein the pharmaceutical composition when orally administered to a patient, provides a medicament plasma concentration-time curve wherein the mean maximum plasma concentration (Cₘₐₓ) in a fasted patient divided by Cₘₐₓ in a fed patient ranges from about 0.20 to about 1.55.

In an embodiment of the present invention, the composition, when orally administered to a patient, provides a medicament plasma concentration time curve wherein the Cₘₐₓ in a fasted patient divided by the Cₘₐₓ in a fed patient ranges from about 0.30 to about 1.55.

In an embodiment of the present invention, the composition, when orally administered to a patient, provides a medicament plasma concentration time curve wherein the Cₘₐₓ in a fasted patient divided by the Cₘₐₓ in a fed patient ranges from about 0.50 to about 0.90.

In an embodiment of the present invention, the composition, when orally administered to a patient, provides a plasma concentration-time curve wherein the Cₘₐₓ of the form of the at least one SSRI in a fasted patient divided by the Cₘₐₓ of the form of the at least one SSRI in a fed patient ranges from about 0.25 to about 1.45.

In an embodiment of the present invention, the composition, when orally administered to a patient, provides a plasma concentration-time curve wherein the Cₘₐₓ of the form of the at least one SSRI in a fasted patient divided by the Cₘₐₓ of the form of the at least one SSRI in a fed patient ranges from about 0.45 to about 0.90.

In accordance with another aspect of the present invention, there is provided a modified release pharmaceutical composition for oral administration, suitable for once daily dosing, comprising about 20 mg of a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition when orally administered to a patient, provides a mean maximum plasma concentration (Cₘₐₓ) of the form of the at least one SSRI from about 6.0 ng/ml to about 28.0 ng/ml.

In an embodiment of the present invention, the composition, when orally administered to a fasting patient, provides a Cₘₐₓ of the form of the at least one SSRI ranging from about 6.0 ng/ml to about 28.0 ng/ml.

In an embodiment of the present invention, the composition, when orally administered to a fed patient, provides a Cₘₐₓ of the form of the at least one SSRI ranging from about 15.0 ng/ml to about 19.0 ng/rn1.

In accordance with another aspect of the present invention, there is provided a modified release pharmaceutical composition for oral administration, suitable for once daily dosing, comprising about 40 mg of a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically acceptable excipient, wherein the composition when orally administered to a patient, provides a mean maximum plasma concentration (Cₘₐₓ) of the form of the at least one SSRI from about 20.0 ng/ml to about 36.0 ng/ml.

In an embodiment of the present invention, the composition, when orally administered to a fasting patient, provides a Cₘₐₓ of the form of the at least one SSRI ranging from about 20.0 ng/ml to about 36.0 ng/ml.

In an embodiment of the present invention, the composition, when orally administered to a fasting patient, provides a Cₘₐₓ of the form of the at least one SSRI ranging from about 26.0 ng/ml to about 30.0 ng/ml.

In an embodiment of the present invention, the composition, when orally administered to a fasting patient, provides a Cₘₐₓ of the form of the at least one SSRI ranging from about 23.0 ng/ml to about 36.0 ng/ml.

In an embodiment of the present invention, the composition, when orally administered to a fasting patient, provides a Cₘₐₓ of the form of the at least one SSRI ranging from about 28.0 ng/ml to about 32.0 ng/ml.

In an embodiment of the present invention, the composition, when orally administered to a fasting patient, provides a Cₘₐₓ of the form of the at least one SSRI ranging from about 22.0 ng/ml to about 36.0 ng/ml.

In an embodiment of the present invention, the composition, when orally administered to a fasting patient, provides a Cₘₐₓ of the form of the at least one SSRI ranging from about 27.0 ng/ml to about 31.0 ng/ml.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition when orally administered to a patient, provides a time to mean maximum plasma concentration (Tₘₐₓ) ranging from about 4 to about 22 hours.

In an embodiment of the present invention, the composition, when orally administered to a fasting patient, provides a Tₘₐₓ ranging from about 4 hours to about 22 hours.

In an embodiment of the present invention, the composition, when orally administered to a fasting patient, provides a T=ranging from about 17 hours to about 21 hours.

In an embodiment of the present invention, the composition, when orally administered to a fasting patient, provides a Tₘₐₓ ranging from about 15 hours to about 19 hours.

In an embodiment of the present invention, the composition, when orally administered to a fasting patient, provides a Tₘₐₓ ranging from about 7 hours to about 11 hours.

In an embodiment of the present invention, the composition, when orally administered to a fasting patient, provides a Tₘₐₓ ranging from about 12 hours to about 16 hours.

In an embodiment of the present invention, the composition, when orally administered to a fasting patient, provides a Tₘₐₓ ranging from about 11 hours to about 15 hours.

In an embodiment of the present invention, the composition, when orally administered to a fasting patient, provides a Tₘₐₓ ranging from about 7 hours to about 13 hours.

In an embodiment of the present invention, the composition, when orally administered to a fasting patient, provides a Tₘₐₓ ranging from about 8 hours to about 12 hours.

In an embodiment of the present invention, the composition, when orally administered to a fasting patient, provides a Tₘₐₓ ranging from about 5 hours to about 11 hours.

In an embodiment of the present invention, the composition, when orally administered to a fasting patient, provides a Tₘₐₓ ranging from about 6 hours to about 10 hours.

In an embodiment of the present invention, the composition, when orally administered to a fed patient, provides a Tₘₐₓ ranging from about 14 to about 21 hours.

In an embodiment of the present invention, the composition, when orally administered to a fed patient, provides a Tₘₐₓ ranging from about 17 hours to about 21 hours.

In an embodiment of the present invention, the composition, when orally administered to a fed patient, provides a Tₘₐₓ ranging from about 14 hours to about 18 hours.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition, when orally administered to a patient, provides a plasma concentration-time curve wherein the time to mean maximum plasma concentration (Tₘₐₓ) of the form of the at least one SSRI in a fasted patient divided by the Tₘₐₓ of the form of the at least one SSRI in a fed patient ranges from about 0.50 to about 1.10.

In an embodiment of the present invention, the composition when orally administered to a patient, provides a medicament plasma concentration-time curve wherein the Tₘₐₓ in a fasted patient divided by the Tₘₐₓ in a fed patient ranges from about 0.50 to about 0.95.

In an embodiment of the present invention, the composition when orally administered to a patient, provides a medicament plasma concentration-time curve wherein the Tₘₐₓ in a fasted patient divided by the Tₘₐₓ in a fed patient ranges from about 0.70 to about 0.80.

In an embodiment of the present invention, the composition when orally administered to a patient, provides a medicament plasma concentration-time curve wherein the Tₘₐₓ in a fasted patient divided by the T in a fed patient ranges from about 0.60 to about 1.10.

In an embodiment of the present invention, the composition when orally administered to a patient, provides a medicament plasma concentration-time curve wherein the Tₘₐₓ in a fasted patient divided by the Tₘₐₓ in a fed patient ranges from about 0.80 to about 0.85.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically acceptable excipient, wherein the composition when orally administered to a patient, provides a time to mean maximum plasma concentration (Tₘₐₓ) ranging from about 4 to about 9 hours.

In an embodiment of the present invention, the composition, when orally administered to a fasting patient, provides a Tₘₐₓ ranging from about 4 hours to about 9 hours.

In an embodiment of the present invention, the composition, when orally administered to a fasting patient, provides a Tₘₐₓ ranging from about 5 hours to about 8 hours.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition when orally administered to a patient, provides a time to mean maximum plasma concentration (Tₘₐₓ) ranging from about 5 to about 14 hours.

In an embodiment of the present invention, the composition, when orally administered to a fasting patient, provides a Tₘₐₓ ranging from about 5 hours to about 14 hours.

In an embodiment of the present invention, the composition, when orally administered to a fasting patient, provides a Tₘₐₓ ranging from about 7 hours to about 13 hours.

In an embodiment of the present invention, the composition, when orally administered to a fasting patient, provides a Tₘₐₓ ranging from about 8 hours to about 12 hours.

In an embodiment of the present invention, the composition, when orally administered to a fasting patient, provides a Tₘₐₓ ranging from about 6 hours to about 10 hours.

In an embodiment of the present invention, the composition, when orally administered to a fasting patient, provides a Tₘₐₓ ranging from about 5 hours to about 12 hours.

In accordance with another aspect of the present invention, there is provided a modified release pharmaceutical composition for oral administration, suitable for once daily dosing, comprising about 20 mg of a form of at least one selective serotonin re-uptake inhibitor (SSRI) selected from the group consisting of SSRIS, racemic mixtures thereof, enantiomers thereof, pharmaceutically acceptable salts thereof and combinations thereof in combination with at least one pharmaceutically-acceptable excipient, wherein the composition when orally administered to a patient provides a plasma concentration time curve with an area under the curve from zero to infinity (AUC_{(0-inf)}) ranging from about 400 ng.hr/ml to about 1500 ng.hr/ml.

In an embodiment of the present invention, wherein the composition, when orally administered to a fasting patient, provides a plasma concentration time curve with an AUC_{(0-inf)} ranging from about 400 ng hr/ml to about 1500 ng.hr/ml.

In an embodiment of the present invention, the composition, when orally administered to a fasting patient, provides a plasma concentration time curve with an AUC_{(0-inf)} ranging from about 400 ng.hr/ml to about 800 ng.hr/ml.

In an embodiment of the present invention, the composition, when orally administered to a fasting patient, provides a plasma concentration-time curve of the form of the at least one SSRI with an AUC_{(0-inf)} ranging from about 500 ng.hr/ml to about 900 ng hr/ml.

In an embodiment of the present invention, the composition, when orally administered to a fasting patient, provides a plasma concentration-time curve of the form of the at least one SSRI with an AUC_{(0-inf)} ranging from about 600 ng.hr/ml to about 1000 ng.hr/ml. In an embodiment of the present invention, the composition, when orally administered to a fasting patient, provides a plasma concentration-time curve of the form of the at least one SSRI with an AUC_{(0-inf)} ranging from about 700 ng.hr/ml to about 1100 ng.hr/ml.

In an embodiment of the present invention, the composition, when orally administered to a fasting patient, provides a plasma concentration-time curve of the form of the at least one SSRI with an AUC_{(0-inf)} ranging from about 800 ng.hr/ml to about 1200 ng.hr/ml.

In an embodiment of the present invention, the composition, when orally administered to a fasting patient, provides a plasma concentration-time curve of the form of the at least one SSRI with an AUC_{(0-inf)} ranging from about 900 ng.hr/ml to about 1300 ng.hr/ml.

In an embodiment of the present invention, the composition, when orally administered to a fasting patient, provides a plasma concentration-time curve of the form of the at least one SSRI with an AUC_{(0-inf)} ranging from about 1000 ng.hr/ml to about 1400 ng.hr/ml.

In an embodiment of the present invention, the composition, when orally administered to a fasting patient, provides a plasma concentration-time curve of the form of the at least one SSRI with an AUC_{(0-inf)} ranging from about 1100 ng.hr/ml to about 1500 ng.hr/ml. In an embodiment of the present invention, the composition, when orally administered to a fed patient, provides a plasma concentration-time curve of the form of the at least one SSRI with an AUC_{(0-inf)} ranging from about 600 ng.hr/ml to about 1000 ng.hr/ml.

In an embodiment of the present invention, the composition, when orally administered to a fed patient, provides a plasma concentration-time curve of the form of the at least one SSRI with an AUC_{(0-inf)} ranging from about 700 ng.hr/ml to about 1100 ng.hr/ml.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically acceptable excipient, wherein the pharmaceutical composition, when orally administered to a patient, provides an area under the curve from zero to infinity (AUC_{(0-inf)}) in a fasted patient, divided by AUC_{(0-inf)} in a fed patient ranges from about 0.60 to about 0.80.

In accordance with another aspect of the present invention, there is provided a modified release pharmaceutical composition for oral administration, suitable for once daily dosing, comprising about 20 mg of a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition when orally administered to a patient provides a plasma concentration time curve with an area under the curve (AUC) ranging from about 100 ng.hr/ml to about 1000 ng.hr/ml.

In an embodiment of the present invention, the composition, when orally administered to a fasting patient, provides a plasma concentration-time curve of the form of the at least one SSRI with an AUC ranging from about 100 ng hr/ml to about 1000 ng.hr/ml.

In an embodiment of the present invention, the composition, when orally administered to a fasting patient, provides a plasma concentration-time curve of the form of the at least one SSRI with an AUC ranging from about 200 ng.hr/ml to about 600 ng.hr/ml.

In accordance with another aspect of the present invention, there is provided a modified release pharmaceutical composition for oral administration, suitable for once daily dosing, comprising about 40 mg of a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition when orally administered to a patient provides a plasma concentration time curve with an area under the curve from zero to infinity (AUC_{(0-inf)}) ranging from about 600 ng.hr/ml to about 12000 ng.hr/ml.

In an embodiment of the present invention, the composition, when orally administered to a fasting patient, provides a plasma concentration-time curve of the form of the at least one SSRI with an AUC_{(0-inf)} ranging from about 600 ng.hr/ml to about 12000 ng.hr/ml.

In an embodiment of the present invention, the composition, when orally administered to a fasting patient, provides a plasma concentration-time curve of the form of the at least one SSRI with an AUC_{(0-inf)} ranging from about 600 ng.hr/ml to about 2200 ng.hr/ml. In an embodiment of the present invention, the composition, when orally administered to a fasting patient, provides a plasma concentration-time curve of the form of the at least one SSRI with an AUC_{(0-inf)} ranging from about 1200 ng.hr/ml to about 1600 ng.hr/ml.

In an embodiment of the present invention, the composition, when orally administered to a fasting patient, provides a plasma concentration-time curve of the form of the at least one SSRI with an AUC_{(0-inf)} ranging from about 600 ng.hr/ml to about 1500 ng.hr/ml.

In an embodiment of the present invention, the composition, when orally administered to a fasting patient, provides a plasma concentration-time curve of the form of the at least one SSRI with an AUC_{(0-inf)} ranging from about 800 ng.hr/ml to about 1200 ng.hr/ml.

In an embodiment of the present invention, the composition, when orally administered to a fasting patient, provides a plasma concentration-time curve of the form of the at least one SSRI with an AUC_{(0-inf)} ranging from about 3000 ng.hr/ml to about 12000 ng.hr/ml.

In an embodiment of the present invention, the composition, when orally administered to a fasting patient, provides a plasma concentration-time curve of the form of the at least one SSRI with an AUC_{(0-inf)} ranging from about 5000 ng.hr/ml to about 9000 ng.hr/ml.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof in combinations thereof in combination with at least one pharmaceutically acceptable excipient, wherein the composition, when orally administered to a patient, provides a plasma concentration time curve with an area under the curve from zero to t hours (AUC₍₀₋ₜ₎) ranging from about 400 ng.hr/ml to about 1500 ng.hr/ml.

In an embodiment of the present invention, the composition, when orally administered to a fasting patient, provides a plasma concentration time curve with an AUC₍₀₋ₜ₎ ranging from about 400 ng.hr/ml to about 1500 ng.hr/ml.

In an embodiment of the present invention, the composition, when orally administered to a fasting patient, provides a plasma concentration time curve with an AUC₍₀₋ₜ₎ ranging from about 400 ng.hr/ml to about 800 ng.hr/ml.

In an embodiment of the present invention, the composition, when orally administered to a fasting patient, provides a plasma concentration time curve with an AUC₍₀₋ₜ₎ ranging from about 500 ng.hr/ml to about 900 ng.hr/ml.

In an embodiment of the present invention, the composition, when orally administered to a fasting patient, provides a plasma concentration time curve with an AUC₍₀₋ₜ₎ ranging from about 800 ng.hr/ml to about 1200 ng.hr/ml.

In an embodiment of the present invention, the composition, when orally administered to a fasting patient, provides a plasma concentration time curve with an AUC₍₀₋ₜ₎ ranging from about 600 ng.hr/ml to about 1000 ng.hr/min.

In an embodiment of the present invention, the composition, when orally administered to a fasting patient, provides a plasma concentration time curve with an AUC₍₀₋ₜ₎ ranging from about 700 ng.hr/ml to about 1100 ng.hr/ml.

In an embodiment of the present invention, the composition, when orally administered to a fasting patient, provides a plasma concentration time curve with an AUC₍₀₋ₜ₎ ranging from about 800 ng.hr/ml to about 1200 ng.hr/ml.

In an embodiment of the present invention, the composition, when orally administered to a fasting patient, provides a plasma concentration time curve with an AUC₍₀₋ₜ₎ ranging from about 900 ng.hr/ml to about 1300 ng.hr/ml.

In an embodiment of the present invention, the composition, when orally administered to a fasting patient, provides a plasma concentration time curve with an AUC₍₀₋ₜ₎ ranging from about 1000 ng.hr/ml to about 1400 ng.hr/nl.

In an embodiment of the present invention, the composition, when orally administered to a fasting patient, provides a plasma concentration time curve with an AUC₍₀₋ₜ₎ ranging from about 1100 ng.hr/ml to about 1500 ng.hr/ml.

In an embodiment of the present invention, the composition, when orally administered to a fed patient, provides a plasma concentration time curve with an AUC₍₀₋ₜ₎ ranging from about 600 ng.hr/ml to about 1000 ng.hr/ml.

In an embodiment of the present invention, the composition, when orally administered to a fed patient, provides a plasma concentration time curve with an AUC₍₀₋ₜ₎ ranging from about 700 ng.hr/ml to about 1100 ng.hr/ml.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically acceptable excipient, wherein the pharmaceutical composition, when orally administered to a patient, provides a medicament plasma concentration time curve wherein the area under the curve from zero to t hours (AUC₍₀₋ₜ₎) in a fasted patient divided by the AUC₍₀₋ₜ₎ in a fed patient ranges from about 0.60 to about 0.80.

In accordance with another aspect of the present invention, there is provided a modified release pharmaceutical composition for oral administration, suitable for once daily dosing, comprising about 40 mg of a form of at least one selective serotonin re-uptake inhibitor (SSRI) selected from the group consisting of SSRIs, racemic mixtures thereof, enantiomers thereof, pharmaceutically acceptable salts thereof in combinations thereof in combination with at least one pharmaceutically acceptable excipient, wherein the composition, when orally administered to a patient, provides a plasma concentration time curve with an area under the curve from zero to t hours (AUC₍₀₋ₜ₎) ranging from about 500 ng.hr/ml to about 6500 ng.hr/ml.

In an embodiment of the present invention, the composition, when orally administered to a fasting patient, provides a plasma concentration time curve with an AUC₍₀₋ₜ₎ ranging from about 500 ng.hr/ml to about 6500 ng.hr/ml.

In an embodiment of the present invention, the composition, when orally administered to a fasting patient, provides a plasma concentration time curve with an AUC₍₀₋ₜ₎ ranging from about 500 ng.hr/ml to about 2200 ng.hr/ml.

In an embodiment of the present invention, the composition, when orally administered to a fasting patient, provides a plasma concentration time curve with an AUC₍₀₋ₜ₎ ranging from about 1000 ng.hr/ml to about 1500 ng.hr/ml.

In an embodiment of the present invention, the composition, when orally administered to a fasting patient, provides a plasma concentration time curve with an AUC₍₀₋ₜ₎ ranging from about 500 ng.hr/ml to about 1500 ng.hr/ml.

In an embodiment of the present invention, the composition, when orally administered to a fasting patient, provides a plasma concentration time curve with an AUC₍₀₋ₜ₎ ranging from about 800 ng.hr/ml to about 1200 ng.hr/ml.

In an embodiment of the present invention, the composition, when orally administered to a fasting patient, provides a plasma concentration time curve with an AUC₍₀₋ₜ₎ ranging from about 3000 ng.hr/ml to about 6200 ng.hr/ml.

In an embodiment of the present invention, the composition, when orally administered to a fasting patient, provides a plasma concentration time curve with an AUC₍₀₋ₜ₎ ranging from about 4400 ng.hr/ml to about 4800 ng.hr/ml.

In accordance with another aspect of the present invention, there is provided a modified release pharmaceutical composition for oral administration, suitable for once daily dosing, comprising about 20 mg of a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the composition when orally administered to a patient provides a plasma concentration time curve with an area under the curve from zero to 24 hours (AUC₍₀₋₂₄₎) ranging from about 100 ng.hr/ml to about 500 ng.hr/ml.

In an embodiment of the present invention, the composition, when orally administered to a fasting patient, provides a plasma concentration-time curve of the form of the at least one SSRI with an AUC₍₀₋₂₄₎ ranging from about 100 to about 500 ng.hr/ml.

In an embodiment of the present invention, the composition, when orally administered to a fasting patient, provides a plasma concentration-time curve of the form of the at least one SSRI with an AUC₍₀₋₂₄₎ ranging from about 200 to about 400 ng.hr/ml.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the pharmaceutical composition when orally administered to a patient as a single dose, releases the form of the at least one SSRI in vivo such that the duration over which the plasma level of the form of the at least one SSRI is equal to or greater than 50% of the mean maximum plasma concentration (Cₘₐₓ) is about 20 hours or greater.

In an embodiment of the present invention, the composition, when orally administered to a patient as a single dose, releases the form of the at least one SSRI in vivo such that the duration over which the plasma level of the form of the at least one SSRI is equal to or greater than 50% of the Cₘₐₓ is about 24 hours or greater.

In an embodiment of the present invention, the composition, when orally administered to a patient as a single dose, releases the form of the at least one SSRI in vivo such that the duration over which the plasma level of the form of the at least one SSRI is equal to or greater than 50% of the Cₘₐₓ is about 30 hours or greater.

In an embodiment of the present invention, the composition, when orally administered to a patient as a single dose, releases the form of the at least one SSRI in vivo such that the duration over which the plasma level of the form of the at least one SSRI is equal to or greater than 50% of the Cₘₐₓ is about 40 hours or greater.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the pharmaceutical composition, when orally administered to a patient as a single dose, releases the form of the at least one SSRI in vivo such that the duration over which the plasma level of the form of the at least one SSRI is equal to or greater than 75% of the mean maximum plasma concentration (Cₘₐₓ) is about 6 hours or greater.

In an embodiment of the present invention, the pharmaceutical composition, when orally administered to a patient as a single dose, releases the form of the at least one SSRI in vivo such that the duration over which the plasma level of the form of the at least one SSRI is equal to or greater than 75% of the Cₘₐₓ is about 12 hours or greater.

In an embodiment of the present invention, the pharmaceutical composition, when orally administered to a patient as a single dose, releases the form of the at least one SSRI in vivo such that the duration over which the plasma level of the form of the at least one SSRI is equal to or greater than 75% of the Cₘₐₓ is about 18 hours or greater.

In an embodiment of the present invention, the pharmaceutical composition, when orally administered to a patient as a single dose, releases the form of the at least one SSRI in vivo such that the duration over which the plasma level of the form of the at least one SSRI is equal to or greater than 75% of the Cₘₐₓ is about 30 hours or greater.

In accordance with another aspect of the present invention, there is provided a modified release pharmaceutical composition for oral administration, suitable for once daily dosing, comprising a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically-acceptable excipient, wherein the pharmaceutical composition, when orally administered to a patient releases the form of the at least one SSRI in vivo at steady state such that the plasma level of the form of the at least one SSRI over the 24 hour dosing period is equal to or greater than 50% of the mean maximum plasma concentration (Cₘₐₓ).

In an embodiment of the present invention, the pharmaceutical composition, when orally administered to a patient releases the form of the at least one SSRI in vivo at steady state such that the duration over which the plasma level of the form of the at least one SSRI over the 24 hour dosing period is equal to or greater than 75% of the Cₘₐₓ is about 12 hours or greater.

In accordance with another aspect of the present invention, there is provided a modified release pharmaceutical composition for oral administration, suitable for once daily dosing, comprising a form of at least one selective serotonin reuptake inhibitor selected from the group consisting of SSRIS, racemic mixtures thereof, enantiomers thereof, pharmaceutically acceptable salts thereof and combinations thereof in combination with at least one pharmaceutically acceptable excipient, wherein the pharmaceutical composition, when orally administered to a patient, provides a mean maximum plasma concentration (Cₘₐₓ) which is more than twice the plasma level of said form of said at least one SSRI at about 16 hours after administration of the pharmaceutical composition.

In an embodiment of the present invention, the composition, when orally administered to a patient, provides a Cₘₐₓ which is more than twice the plasma level of said form of said at least one SSRI at about 20 hours after administration of the pharmaceutical composition.

In an embodiment of the present invention, the composition, when orally administered to a patient, provides a Cₘₐₓ which is more than twice the plasma level of said form of said at least one SSRI at about 24 hours after administration of the pharmaceutical composition.

In an embodiment of the present invention, the composition, when orally administered to a patient, provides a Cₘₐₓ which is more than twice the plasma level of said form of said at least one SSRI at about 36 hours after administration of the pharmaceutical composition.

In an embodiment of the present invention, the composition, when orally administered to a patient, provides a Cₘₐₓ which is more than twice the plasma level of said form of said at least one SSRI at about 48 hours after administration of the pharmaceutical composition.

In accordance with another aspect of the present invention, there is provided a method of effectively treating depression in humans, comprising orally administering to a human patient on a once a day basis an oral sustained release dosage form containing a form of at least one selective serotonin re-uptake inhibitor (SSRI) selected from the group consisting of SSRIs, racemic mixtures thereof, enantiomers thereof, pharmaceutically acceptable salts thereof and combinations thereof in combination with at least one pharmaceutically acceptable excipient which upon administration provides a time to maximum plasma concentration (Tₘₐₓ) of said form of said at least one SSRI in about 6 to about 20 hours and a maximum plasma concentration (Cₘₐₓ) which is more than twice the plasma level of said form of said at least one SSRI at about 48 hours after administration of the dosage form, and which dosage form provides effective treatment of depression for about 24 hours or more after administration to the patient. In accordance with another aspect of the present invention there is provided a method of effectively treating depression in a human patient, comprising orally administering to a human patient on a once a day basis an oral sustained release dosage form containing a form of at least one selective serotonin reuptake inhibitor selected from the group consisting of SSRIs, racemic mixtures thereof, enantiomers thereof, pharmaceutically acceptable salts thereof and combinations thereof in combination with at least one pharmaceutically acceptable excipient, which provides a maximum plasma concentration (Cₘₐₓ) which is more than twice the plasma level of said form of said at least one SSRI at about 48 hours after administration of the dosage form, and which provides effective treatment of depression for about 24 hours or more after administration to the patient.

In an embodiment of the present invention, the form of the at least one SSRI is selected from the group consisting of Fluoxetine, Fluvoxamine, Paroxetine, Sertraline, Venlafaxine, Citalopram, racemic mixtures thereof, enantiomers thereof, pharmaceutically acceptable salts thereof and combinations thereof.

In an embodiment of the present invention, the form of the at least SSRI is Citalopram HBr.

In an embodiment of the present invention, the form of the at least SSRI is Escitalopram oxalate.

In an embodiment of the present invention, the form of the at least SSRI is Fluvoxamine maleate.

In an embodiment of the present invention, the form of the at least SSRI is Paroxetine HCl.

In an embodiment of the present invention, the form of the at least SSRI is Sertraline HCl.

In an embodiment of the present invention, the form of the at least SSRI is Venlafaxine HCl.

In an embodiment of the present invention, the form of the at least one SSRI is present in the pharmaceutical composition in an amount effective to treat at least one condition selected from the group consisting of depression, major depressive disorder, obsessive compulsive disorder, panic disorder, social anxiety disorder, generalized anxiety disorder, posttraumatic stress disorder and combinations thereof.

In an embodiment of the present invention, the form of the at least one SSRI is present in the pharmaceutical composition in the range of from about 5 mg to about 1000 mg (calculated as the pharmaceutically-acceptable salt) per dosage unit.

In an embodiment of the present invention the form of the least one SSRI is present in the pharmaceutical composition in an amount in the range of from about 10 mg to about 200 mg (calculated as the pharmaceutically acceptable salt) per dosage unit.

In an embodiment of the present invention, the form of the at least one SSRI is present in the pharmaceutical composition in an amount in the range of from about 10 mg to about 100 mg (calculated as the pharmaceutically acceptable salt) per dosage unit.

In an embodiment of the present invention, the at least one pharmaceutically acceptable excipient is selected from the group comprising at least one release rate controlling pharmaceutically acceptable carrier, at least one diluent, at least one binder, at least one filler, at least one solubility enhancer, at least one bioavailability enhancer, at least one lubricant, at least one solubilizing agent, at least one surface active agent, at least one surfactant, at least one acidifying agent and combinations thereof.

In an embodiment of the present invention, the at least one release rate controlling pharmaceutically acceptable carrier is at least one sustained release pharmaceutically acceptable carrier.

In an embodiment of the present invention, the at least one sustained release pharmaceutically acceptable carrier is at least one solid sustained release pharmaceutically acceptable carrier.

In an embodiment of the present invention, the at least one solid sustained release pharmaceutically acceptable carrier is at least one solid sustained release pharmaceutically-acceptable polymer.

In an embodiment of the present invention, the at least one solid sustained release pharmaceutically-acceptable polymer is selected from the group consisting of at least one hydrophilic water-soluble polymer, at least one hydrophobic water-insoluble polymer and combinations thereof.

Any suitable hydrophilic water-soluble polymer conventional in the pharmaceutical art may be used. Examples of hydrophilic polymers suitable for use in the present invention include, but are not limited to, cellulose derivatives, dextrans, starches, carbohydrates, base polymers, natural or hydrophilic gums, xanthans, alginates, gelatins, polyacrylic acids, polyvinyl alcohol (PVA), polyvinyl pyrrolidone (PVP), carbomers or the like. The hydrophilic polymers can be used individually, as well as in mixtures of two or several hydrophilic polymers. In the case of the cellulose derivatives, the alkyl or hydroxyalkyl cellulose derivatives, the alkyl or hydroxyalkyl cellulose derivatives preferably come into consideration such as example, methyl cellulose, ethylcellulose (EC), hydroxy methylcellulose, hydroxyethyl cellulose (HEC), hydroxypropyl cellulose (HPC), hydroxypropyl methylcellulose (HPMC), methylhydroxy ethylcellulose, methylhydroxy ethylcellulose, methylhydroxy propylcellulose or sodium carboxymethyl cellulose.

Suitable cellulose based hydrophilic polymers may have various degrees of substitution and/or different molecular weights corresponding to a different degree of viscosity of the aqueous solution.

In an embodiment of the present invention, the release rate controlling polymer may be selected from the group consisting of hydroxypropylmethyl cellulose, hydroxyethyl cellulose, ethylcellulose, carbomer and combinations thereof.

The hydroxypropyl methylcellulose (HPMC) used as the release rate controlling polymer in the present invention may suitably be any HPMC conventional in the pharmaceutical art. The HPMC used may suitably be, for example, HPMC substitution types 1828, 2208, 2906 and 2910 as described on page 843 of the 24^{th} Edition (2000) of the United States Pharmacopeia (USP XXIV). The hydroxypropyl methylcellulose used may suitably be, for example, METHOCEL® as supplied by Dow Chemical Company. Similar HPMCs are also available from other suppliers. Preferably, the HPMC used is HPMC 2208, more preferably METHOCEL® K4M Premium CR.

The ethylcellulose (EC) used as the release rate controlling polymer in the present invention may suitably be any EC conventional in the pharmaceutical art. The EC used may suitably be, for example, ETHOCEL® as supplied by Dow Chemical Company. Similar ECs are also available from other suppliers. Preferably, the EC used is EIHOCEL® FP, more preferably ETHOCEL® FP 100.

The carbomer used as the release rate controlling polymer in the present invention may suitably be any carbomer conventional in the pharmaceutical art. The carbomer used may suitably be, for example, carbomer 910, carbomer 934 and 934P, carbomer 941 and carbomer 1342 as described on pages 2426 to 2428 of the 19^{th} Edition (2000) of the United States National Formulary (USNFXIX). The carbomer used may suitably be, for example, CARBOPOL® as supplied by B.F. Goodrich. Similar carbomers are also available from other, suppliers. Preferably, the carbomer used is carbomer 941, more preferably CARBOPOL® 971P.

In an embodiment of the present invention, the modified release pharmaceutical composition may also comprise other pharmaceutically acceptable ingredients which are conventional in the pharmaceutical arts, such as at least one diluent, at least one lubricant, at least one binder, at least one granulating aid, at least one colourant, at least one flavourant, at least one surfactant, at least one pH adjuster, at least one anti-adherent, at least one glidant, at least one disintegrant, at least one solubility enhancer, at least one bioavailability enhancer, at least one solubilizing agent, at least one surface active agent, at least one surfactant and the like and combinations thereof.

In an embodiment of the present invention, the pharmaceutical composition may further comprise at least one diluent. Any suitable diluent conventional in the pharmaceutical art may be used. Examples of diluents suitable for use in the present invention include, but are not limited to, lactose, microcrystalline cellulose, mannit61 and combinations thereof. The lactose used may suitably be lactose anhydrous (direct tabletting) as supplied by Quest International. The microcrystalline cellulose used may suitably bes for example, AVICEL® as supplied by FMC Corporation, preferably AVICEL® PH 101 or AVICEL® PH 102.

In an embodiment of the present invention, the pharmaceutical composition may further comprise at least one binder. Any suitable binder conventional in the pharmaceutical art may be used. An example of a suitable binder for use in the present invention is polyvinyl pyrrolidone, for example KOLLIDON® as supplied by BASF AG, preferably KOLLIDON® 29/32 and/or KOLLIDON® 90F.

In an embodiment of the present invention, the pharmaceutical composition may further comprise at least one lubricant. Any suitable lubricant conventional in the pharmaceutical art may be used. Examples of suitable lubricants for use in the present invention include, but are not limited to, magnesium stearate, stearic acid and combinations thereof.

In an embodiment of the present invention, the pharmaceutical composition may further comprise at least one surfactant. Any surfactant conventional in the pharmaceutical art may be used. Examples of suitable surfactants for use in the present invention include, but are not limited to, a bile salt, sodium lauryl sulphate (SLS), polyoxyethylene/polyoxypropylene block copolymers, polyethylene glycol hydrogenated castor oils, polyethylene glycols, saturated polyglycolized glycerides from hydrogenated vegetable oils, saturated polyglycolized glycerides, water soluble derivatives of natural source vitamins, sucrose stearate, mannitol, mono- and diglycerides. The polyoxyethylene/polyoxypropylene block polymers used as the surfactant in the present invention may suitably be, for example, poloxamers, preferably poloxamer 407 or poloxamer 188 such as LUTROL® F127 or LUTROL® F68, respectively as supplied by BASF AG. The polyethylene ethylene glycol hydrogenated castor oils used as the surfactant in the present invention may suitably be, for example, PEG40 hydrogenated castor oil, such as CREMOPHOR® RH40 as supplied by BASF AG. The polyethylene glycols used as the surfactant in the present invention may suitably be, for example, PEG-3350, PEG-600, PEG8000 such as CARBOWAX® as supplied by Union Carbide and PEG-32 such as LUTROL® E1500 as supplied by BASF AG. The saturated polyglycolized glycerides from hydrogenated vegetable oils used as the surfactant in, the present invention may suitably be, for example, lauroyl macrogol-32 glycerides such as GELUCIRE® 44/14 as supplied by Gattefosse SA. The saturated polyglycolized glycerides used as the surfactant in the present invention may suitably be, for example, stearoyl macrogol-32 glycerides such as GELUCIRE® 50/13 as supplied by Gattefosse SA. The water soluble derivatives of natural source vitamins used as the surfactant in the present invention may suitably be, for example, Vitamin E d-∝-tocopheryl polyethylene glycol. 1000 succinate (IPGS) as supplied by Eastman. The sucrose stearate used as the surfactant in the present invention may suitably be, for example, CRODESTA® F160 as supplied by Croda Inc., The mono- and diglycerides used as the surfactant in the present invention may suitably be, for example, a propylene glycol monoester of medium chain fatty acids such as CAPMUL® PG8 as supplied by Abitec Corporation.

In an embodiment of the present invention, the pharmaceutical composition may further comprise at least one acidifying agent. Any acidifying agent conventional in the pharmaceutical art may be used. An example of a suitable acidifying agent for use in the present invention includes, but is not limited to, L-tartaric acid.

In an embodiment of the present invention, the form of the at least one SSRI may be incorporated into a matrix. The matrix may be any matrix conventional in the pharmaceutical art.

In an embodiment of the present invention the matrix is a modified release matrix that affords modified release of the form of the at least one SSRI over at least a 12 hour period and preferably that affords in vitro dissolution rates and in vivo absorption rates of the form of the at least one SSRI within the ranges specified above.

In an embodiment of the present invention, the matrix is selected from the group consisting of a sustained release matrix and a controlled release matrix.

In an embodiment of the present invention wherein the matrix is a sustained release matrix or a controlled release matrix, the pharmaceutical composition further comprises a modified release coating.

In an embodiment of the present invention, the matrix is a normal release matrix having a coating which provides for modified release of the form of the at least one SSRI.

In an embodiment of the present invention, the pharmaceutical composition further comprises a film coating. Any film coating material conventional in the pharmaceutical art may be used. Preferably, an aqueous film coating is used. The film coating functions to seal all surface pores and to provide a smooth and uniform surface. The film coat is obtained by preferably spray coating film coating dispersions onto the surface of uncoated cores using appropriate coating equipment. Usually these dispersions contain low viscosity hydrophilic polymers such as hydroxypropyl methylcellulose and hydroxypropyl cellulose, and plasticizers such as polyethylene glycol 400. These dispersions are commercially available as OPADRY® from Colorcon, West Point, Pa. Similar film coating dispersions are also available from other suppliers. Preferably the optional seal coating membrane is present in a concentration of about 0-5% W/W of the core. An example of a suitable film coating for use in the present invention is OPADRY® II White, preferably OPADRY® II White Y-22-7719. OPADRY® II White Y-22-7719 consists of hydroxypropylmethyl cellulose, titanium dioxide, polydextrose, triacetin and polyethylene glycol. Similar film coating materials are also available from other suppliers.

In an embodiment of the present invention the pharmaceutical composition further comprises a modified-release film coating. Any modified-release film coating material conventional in the pharmaceutical art may be used. The modified release film coating is applied to pharmaceutical products in order to modify drug release. There are two types of modified-release dosage forms, namely those that are delayed release and those that are extended release. Delayed-release products often are designed to prevent drug release in the upper part of the gastrointestinal (GI) tract. Modified release film coatings used to prepare this type of dosage form are commonly called enteric coatings. Extended-release products are designed to extend drug release over a period of time, a result which can be achieved by the application of a sustained- or controlled-release film coating. Modified release film coating is obtained by preferably spraying modified release film coating dispersions onto the surface of seal coated cores. However, these modified release film coating dispersions can also be coated straight onto the surface of the uncoated cores. In an embodiment of the present invention, the modified release film coating comprises as an aqueous dispersion, preferably with appropriate coating ingredients dispersed therein.

In an embodiment of the present invention, the modified release film coating further comprises plasticizers, film extenders, diffusion enhancers and other excipients such as detackifiers or opacifiers, etc. The hydrophobic polymer is mixed with a film extender/diffusion enhancer to give the hydrophobic polymer some degree of hyrophilicity. The plasticizer is added to reduce the glass transition temperature (T_{g}) of the polymer so that it can be coalesced at a lower temperature (such as 60° C.). The plasticizer also makes the functional coating membrane flexible so that it can stretch to some degree without breaking. Preferably, the ratio of the polymer to film extender in the aqueous polymeric dispersion of the functional coating membrane is from about 0.25-0.75 to 0.99-0.01.

The aqueous dispersions of hydrophobic polymers used as modified release film coatings in the present invention may be used in conjunction with tablets, spheroids (or beads), microspheres, seeds, pellets, ion-exchange resin beads, and other multi-particulate systems in order to obtain a desired controlled-release of the therapeutically active agent. Granules, spheroids, or pellets, etc., prepared in accordance with the present invention can be presented in a tablet, a capsule or in any other suitable dosage form. The tablets of the present invention may be any suitable shape, such as round, oval, biconcave, hemispherical, any polygonal shape such as square, rectangular, and pentagonal, and the like.

In order to obtain a modified-release formulation, it is usually necessary to overcoat the pharmaceutical composition with a sufficient amount of the aqueous dispersion of the hydrophobic polymer, to obtain a weight gain level from about 2 to about 25%, although the overcoat may be lesser or greater depending upon the physical properties of the form of the at least one SSRI and the desired release rate, the inclusion of plasticizer in the aqueous dispersion and the manner of incorporation of the same, for example.

In an embodiment of the present invention, the hydrophobic polymer is selected from the group consisting of ethylcellulose, an acrylic polymer and combinations thereof.

Although ethylcellulose is one preferred hydrophobic polymer which may be used for coating the pharmaceutical composition of the present invention, those skilled in the art will appreciate that other cellulosic polymers, including other alkyl cellulosic polymers, may be substituted for part or all of the ethylcellulose included in the hydrophobic polymer coatings of the present invention.

One commercially-available aqueous dispersion of ethylcellulose is AQUACOAT® (FMC Corp., Philadelphia, Pa., U.S.A.). AQUACOAT® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the AQUACOAT® with a suitable plasticizer prior to use.

Another aqueous dispersion of ethylcellulose is commercially available as SURELEASE® (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly into substrates.

Examples of pharmaceutically-acceptable acrylic polymers suitable for use in the present invention, include but are not limited to acrylic acid and methacrylic acid copolymers, methacrylic acid copolymers, methyl methacrylate copolymers, ethoxyethyl methacrylates, cyan oethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers.

In order to obtain a desirable dissolution profile, it may be necessary to incorporate two or more pharmaceutically-acceptable acrylic polymers having differing physical properties.

In an embodiment of the present invention, the modified-release coating is an enteric coating. An example of a suitable enteric coating material for use in the present invention is a methacrylic acid copolymer. The methacrylic acid copolymers used may suitably be any methacrylic acid copolymer conventional in the pharmaceutical art such as methacrylic acid copolymer Types A, B and C as described on page 2477 to page 2479 of USNFXIX.

In an embodiment of the present invention, the hydrophobic acrylic polymer is a polymer whose permeability is pH dependent, such as anionic polymers synthesized from methacrylic acid and methacrylic acid methyl ester. Such polymers are commercially available, e.g., from Rohm Pharma GmbH under the tradename EUDRAGIT® L and EUDRAGIT® S. The ratio of free carboxyl groups to the esters is said to be 1:1 in EUDRAGIT® L and 1:2 in EUDRAGIT® S. EUDRAGIT® L is insoluble in acids a pure water, but becomes increasingly permeable above pH 5.0 EUDRAGIT® S is similar, except that it becomes increasingly permeable above pH 7. Preferably, the methacrylic acid copolymer is methacrylic acid copolymer Type A and/or Type B, more preferably EUDRAGIT® L100 and/or EUDRAGIT® S100.

In one preferred embodiment, the acrylic coating is an acrylic resin lacquer used in the form of an aqueous dispersion, such as that which is commercially available from Rohm Pharma under the Tradename EUDRAGIT®. In further preferred embodiments, the acrylic coating comprises a mixture of two acrylic resin lacquers commercially available from Rohm Pharma under the Tradenames EUDRAGIT® L100 and EUDRAGIT® S100, respectively.

EUDRAGIT® L100 and EUDRAGIT® S100 are anionic copolymers based on methacrylic acid and methyl methacrylate. The ratio of free carboxyl groups to the ester groups is about 1:1 in EUDRAGIT® L100 and about 1:2 in EUDRAGIT® S100. The average molecular weight is about 135,000. The films are insoluble below pH5 and thus resistant to gastric fluid. By salt formation in the neutral to weakly alkaline medium of intestinal fluid, the films dissolve step-wise at pH values above 5.5.

The EUDRAGIT® L/S dispersions of the present invention may be mixed together in any desired ratio in order to ultimately obtain a controlled-release formulation having a desirable dissolution profile. Desirable controlled-release formulations may be obtained, for instance, from an enteric coating derived from 100% EUDRAGIT® L100, 100% EUDRAGIT® S100, 50% EUDRAGIT® L100 and 50% EUDRAGIT® S100, and 90% EUDRAGIT® L100 and 10% EUDRAGIT® S100. Of course, one skilled in the art will recognize that other acrylic polymers may also be used.

In an embodiment of the present invention wherein the modified release film coating comprises an aqueous dispersion of a hydrophobic polymer, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate, tributyl citrate, dibutyl phthalate, and possibly 1,2-propylene glycol. Other plasticizers which have proved to be suitable for enhancing the elasticity of the films formed from acrylic films such as EUDRAGIT® L/S lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

It has further been found that the addition of a small amount of talc reduces the tendency of the aqueous dispersion to stick during processing, and acts as a polishing agent.

In addition to modifying the dissolution profile by altering the relative amounts of different acrylic resin lacquers, the dissolution profile of the ultimate product may also be modified, for example, by increasing or decreasing the thickness of the retardant coating.

In an embodiment of the present invention, the at least one selective SSRI is selected from the group consisting of Fluoxetine HCl, Fluvoxamine maleate, Paroxetine HCl, Sertraline HCl, Venlafaxine HCl, Citalopram HBr, Escitalopram oxalate and combinations thereof.

In accordance with another aspect of the present invention, there is provided a modified release pharmaceutical composition comprising:

| | Ingredients | % w/w |
|---|---|---|
| (a) | SSRI | 4.0-8.0 |
| (b) | Hydroxypropylmethylcellulose 2208 | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinylpyrrolidone (KOLLIDON ® 29/32) | 0.0-4.0 |
| (f) | Polyvinylpyrrolidone (KOLLIDON ® K90F) | 0.0-4.0 |
| (g) | Magnesium Stearate | 0.0-2.0 |
| (h) | Stearic Acid | 0.0-2.0 |
| (i) | Carbomer 941 (CARBOPOL ® 971P) | 0.0-2.0 |
| (j) | Bile Salt | 0.0-3.0 |
| (k) | Sodium Lauryl Sulphate | 0.0-3.0 |
| (l) | Poloxamer 407 (LUTROL ® F127) | 0.0-6.0 |
| (m) | Poloxamer 188 (LUTROL ® F68) | 0.0-6.0 |
| (n) | PEG-40 hydrogenated castor oil | 0.0-6.0 |
| | (CREMOPHOR ® RH40) | |
| (o) | PEG-3350 | 0.0-6.0 |
| (p) | PEG-600 | 0.0-6.0 |
| (q) | PEG-8000 | 0.0-6.0 |
| (r) | Saturated Polyglycolized Glycerides from | 0.0-6.0 |
| | hydrogenated vegetable oils (GELUCIRE ® 44/14) | |
| (s) | Saturated Polyglycolized Glycerides | 0.0-6.0 |
| | (GELUCIRE ® 50/13) | |
| (t) | Vitamin E TPGS | 0.0-6.0 |
| (u) | PEG-32 (LUTROL ® E1500) | 0.0-6.0 |
| (v) | Sucrose stearate (CRODESTA ® F160) | 0.0-6.0 |
| (w) | Mannitol | 0.0-6.0 |
| (x) | Propylene glycol monoester of medium chain fatty | 0.0-6.0 |
| | acids (CAPMUL ® PG8) | |
| (y) | L-Tartaric Acid | 0.0-6.0 |

In accordance with another aspect of the present invention there is provided a modified release pharmaceutical composition comprising:

| | Ingredients | % w/w |
|---|---|---|
| (a) | SSRI | 4.0-8.0 |
| (b) | Hydroxypropylmethyl cellulose | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinylpyrrolidone (KOLLIDON ® K90F) | 0.5-3.50 |
| (f) | Magnesium Stearate | 0.5-2.0 |

In accordance with another aspect of the present invention there is provided a modified release pharmaceutical composition comprising:

| | Ingredients | % w/w |
|---|---|---|
| (a) | Citalopram HBr | 4.0-8.0 |
| (b) | Hydroxypropylmethylcellulose 2208 | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinylpyrrolidone (KOLLIDON ® 29/32) | 0.0-4.0 |
| (f) | Polyvinylpyrrolidone (KOLLIDON ® K90F) | 0.0-4.0 |
| (g) | Magnesium Stearate | 0.0-2.0 |
| (h) | Stearic Acid | 0.0-2.0 |
| (i) | Carbomer 941 (CARBOPOL ® 971P) | 0.0-2.0 |
| (j) | Bile Salt | 0.0-3.0 |
| (k) | Sodium Lauryl Sulphate | 0.0-3.0 |
| (l) | Poloxamer 407 (LUTROL ® F127) | 0.0-6.0 |
| (m) | Poloxamer 188 (LUTROL ® F68) | 0.0-6.0 |
| (n) | PEG-40 hydrogenated castor oil | 0.0-6.0 |
| | (CREMOPHOR ® RH40) | |
| (o) | PEG-3350 | 0.0-6.0 |
| (p) | PEG-600 | 0.0-6.0 |
| (q) | PEG-8000 | 0.0-6.0 |
| (r) | Saturated Polyglycolized Glycerides from | 0.0-6.0 |
| | hydrogenated vegetable oils (GELUCIRE ® 44/14) | |
| (s) | Saturated Polyglycolized Glycerides | 0.0-6.0 |
| | (GELUCIRE ® 50/13) | |
| (t) | Vitamin E TPGS | 0.0-6.0 |
| (u) | PEG-32 (LUTROL ® E1500) | 0.0-6.0 |
| (v) | Sucrose stearate (CRODESTA ® F160) | 0.0-6.0 |
| (w) | Mannitol | 0.0-6.0 |
| (x) | Propylene glycol monoester of medium chain fatty | 0.0-6.0 |
| | acids (CAPMUL ® PG8) | |
| (y) | L-Tartaric Acid | 0.0-6.0 |

In accordance with another aspect of the present invention there is provided a modified release pharmaceutical composition comprising:

| | Ingredients | % w/w |
|---|---|---|
| (a) | Citalopram HBr | 4.0-8.0 |
| (b) | Hydroxypropylmethyl cellulose | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinylpyrrolidone (KOLLIDON ® K90F) | 0.5-3.50 |
| (f) | Magnesium Stearate | 0.5-2.0 |

In accordance with another aspect of the present invention there is provided a modified release pharmaceutical composition comprising:

| | Ingredients | % w/w |
|---|---|---|
| (a) | Paroxetine HCl | 4.0-8.0 |
| (b) | Hydroxypropylmethyl cellulose | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinylpyrrolidone (KOLLIDON ® K90F) | 0.5-3.50 |
| (f) | Magnesium Stearate | 0.5-2.0 |

In accordance with another aspect of the present invention there is provided a modified release pharmaceutical composition comprising:

| | Ingredients | % w/w |
|---|---|---|
| (a) | Fluoxetine HCl | 4.0-8.0 |
| (b) | Hydroxypropylmethyl cellulose | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinylpyrrolidone (KOLLIDON ® K90F) | 0.5-3.50 |
| (f) | Magnesium Stearate | 0.5-2.0 |

In an embodiment of the present invention, the pharmaceutical composition is a matrix tablet.

In an embodiment of the present invention, a wet granulation method issued in the manufacture of the pharmaceutical composition, followed by drying, milling and blending of lubricants before tabletting.

### BRIEF DESCRIPTION OF THE DRAWINGS

The present invention will be further understood from the following detailed description with reference to the drawings in which:

FIG. 1 is a graph illustrating the in vitro dissolution profiles of 20 mg Citalopram HBr CR Tablets formulated according to three different embodiments of the present invention.

FIG. 2 is a graph illustrating the comparison of the mean plasma Citalopram concentrations (ng/ml) over time after a single dose of one of the three 20 mg Citalopram HBr CR Tablets of FIG. 1 or after a single dose of one 20 mg CELEXA™ tablet under fasting conditions.

FIG. 3 is a graph illustrating the in vitro dissolution profiles of 20 mg Citalopram HBr CR Tablets formulated according to two different embodiments of the present invention.

FIG. 4 is a graph illustrating the mean plasma Citalopram concentrations (ng/ml) over time after a single dose of one of two 20 mg Citalopram HBr CR Tablets of FIG. 3 under fasting or fed conditions.

FIG. 5 is a graph illustrating the in vitro dissolution profiles of 20 mg Citalopram HBr CR Tablets formulated according to three different embodiments of the present invention.

FIG. 6 is a graph illustrating the in vitro dissolution profiles of 20 mg Citalopram HBr CR Tablets formulated according to ten different embodiments of the present invention.

FIG. 7 is a graph illustrating the in vitro dissolution profiles of 20 mg Citalopram HBr 20 mg CR Tablets formulated according to sixteen different embodiments of the present invention.

FIG. 8 is, a graph illustrating the in vitro dissolution profiles of 20 mg Citalopram HBr CR Tablets formulated according to two different embodiments of the present invention.

FIG. 9 is a graph illustrating the in vitro dissolution profiles of 20 mg Citalopram HBr CR Tablets formulated according to two different embodiments of the present invention.

FIG. 10 is a graph illustrating the in vitro dissolution profiles of 20 mg Citalopram HBr CR Tablets formulated according to two different embodiments of the present invention.

FIG. 11 is a graph illustrating the mean plasma Citalopram concentrations (ng/ml) over time after a single dose of one of the two 20 mg Citalopram HBr CR Tablets of FIG. 10 or after a single dose of one 20 mg CELEXA™ tablet under fasting conditions.

FIG. 12 is a graph illustrating the in vitro dissolution profile of 20 mg Citalopram HBr CR Tablets formulated according to one embodiment of the present invention.

FIG. 13 is a graph illustrating the mean plasma Citalopram concentrations (ng/ml) over time after a single dose of the 20 mg Citalopram HBr CR Tablet of FIG. 12 or after a single dose of one 20 mg CELEXA™ tablet under fasting conditions.

FIG. 14 is a graph illustrating the mean plasma Desmethylcitalopram concentrations (ng/ml) over time after a single dose of the 20 mg Citalopram HBr CR Tablet of FIG. 12 or after a single dose of one 20 mg CELEXA™ tablet under fasting conditions.

FIG. 15 is, a graph illustrating the mean plasma Didesmethylcitalopram concentrations (ng/ml) over time after a single dose of the 20 mg Citalopram HBr CR Tablet of FIG. 12 or after a single dose of one 20 mg CELEXA™ tablet under fasting conditions.

FIG. 16 is a graph illustrating the in vitro dissolution profiles of 20 mg Citalopram HBr CR Tablets formulated according to an embodiment of the present invention wherein the Citalopram HBr is provided by two different suppliers.

FIG. 17 is a graph illustrating the in vitro dissolution profile of 20 mg Paroxetine HCl CR Tablets formulated according to an embodiment of the present invention.

FIG. 18 is a graph illustrating the mean plasma Paroxetine concentrations (ng/1 ml) over time after a single dose of the 20 mg Paroxetine HCl CR Tablet of FIG. 17 or after a single dose of one 20 mg CELEXA™ tablet under fasting conditions.

FIG. 19 is a graph illustrating the in vitro dissolution profiles of 40 mg Fluoxetine HCl CR Tablets formulated according to an embodiment of the present invention wherein the Fluoxetine HCl is provided by two different suppliers.

FIG. 20 is a graph illustrating the mean plasma Fluoxetine concentrations (ng/ml) over time after a single dose of one of the 40 mg Fluoxetine HCl CR Tablets of FIG. 19 or after a single dose of one 40 mg PROZAC® PULVULE® under fasting conditions.

FIG. 21 is a graph illustrating the mean plasma Norfluoxetine concentrations (ng/ml) over time after a single dose of one of the 40 mg Fluoxetine HCl CR Tablet of FIG. 19 or after a single dose of one 40 mg. PROZAC® PULVULE® under fasting conditions.

FIG. 22 is a graph illustrating the mean plasma Fluoxetine concentrations (ng/ml) over time after a single dose of one of the 40 mg Fluoxetine HCl CR Tablet of FIG. 19 or after a single dose of one 40 mg PROZAC® PULVULE® under fasting conditions.

FIG. 23 is a graph illustrating the mean plasma Norfluoxetine concentrations (ng/ml) over time after a single dose of one of the 40 mg Fluoxetine HCl CR Tablet of FIG. 19 or after a single dose of one 40 mg PROZAC® PULVULE® under fasting conditions.

FIG. 24 is a graph illustrating the mean plasma Fluoxetine concentrations (ng/ml) over time after a single dose of one of the 40 mg Fluoxetine HCl CR Tablet of FIG. 19 or after a singe dose of one 40 mg PROZAC® PULVULE® under fasting conditions.

FIG. 25 is a graph illustrating the mean plasma Norfluoxetine concentrations (ng/ml) over time after a singe dose of one of the 40 mg Fluoxetine HCl CR Tablet of FIG. 19 or after a single dose of one 40 mg PROZAC® PULVULE® under fasting conditions.

FIG. 26 is a schematic diagram illustrating the manufacture of SSRI CR Tablets according to one embodiment of the present invention.

### DETAILED DESCRIPTION OF THE PREFERRED EMBODIMENTS

Further details of the preferred embodiments of the present invention are illustrated in the following examples which are understood to be non-limiting.

### Example 1

Table 1 provides the composition of four Citalopram HBr Controlled Release (CR) Tablet formulations (Formulations 1, 2, 3 and 4).

TABLE 1
Citalopram HBr CR Tablet Formulations

| | Formulation 1 | Formulation 2 | Formulation 3 | Formulation 4 |
|---|---|---|---|---|
| | (g) | (g) | (g) | (g) |
| CORE INGREDIENTS | | | | |
| Citalopram HBr | 187.6 | 187.5 | 187.5 | 187.5 |
| HPMC2208 (METHOCEL ® | 1380.1 | 1350.1 | 1050.0 | 1350.0 |
| K4M Premium CR) | | | | |
| HEC (NATROSOL ® 250 HHX) | 780.2 | 600.0 | NA | 600.0 |
| Lactose Anhydrous (DT) | 240.1 | 360.0 | 1440.1 | 360.0 |
| MCC (AVICEL ® PH 101) | 210.2 | 240.0 | 240.0 | 240.0 |
| Carbomer 941 (CARBOPOL ® | 112.6 | NA | NA | NA |
| 971P) | | | | |
| EC (ETHOCEL ® 100 FP | NA | 1800.0 | NA | 180.0 |
| Premium) | | | | |
| PVP (KOLLIDON ® 29/32) | 60.1 | 120.5 | 52.5 | 120.5 |
| Magnesium stearate | 30.1 | 30.0 | 30.0 | 30.0 |
| Isopropyl Alcohol* | — | — | — | — |
| COATING INGREDIENTS | | | | |
| Film Coating | | | | |
| OPADRY ® II White Y-22-7719 | 30.0 | 30.0 | 30.0 | NA |
| Enteric Coating | | | | |
| Methacrylic Acid Copolymer, | NA | NA | NA | 35.75 |
| Type A (EUDRAGIT ® L 100) | | | | |
| Methacrylic Acid Copolymer, | NA | NA | NA | 3.9 |
| Type B (EUDRAGIT ® S 100) | | | | |
| Ethanol | NA | NA | NA | 650.0 |
| PEG 600 | NA | NA | NA | 7.8 |
| Talc | NA | NA | NA | 6.5 |
| Titanium dioxide | NA | NA | NA | 1.6 |
| Iron Oxide | NA | NA | NA | 2.6 |

*Isopropyl Alcohol is not considered as part of the theoretical batch size since it is evaporated during drying of the wet granulation

As can be seen from Table 1, Formulations 1, 2 and 3 comprise an OPADRY® film coating, whereas Formulation 4 comprises an EUDRAGIT® enteric coating.

In vitro dissolution studies were conducted on 20 mg Citalopram HBr CR Tablets formulated according to Formulations 1, 2 and 3. The following dissolution conditions were used for all of the in vitro dissolution studies conducted herein for determining the in vitro dissolution profiles of SSRI CR

### Tablets:

### Apparatus

USP Dissolution Apparatus #2 (Paddle)

### Sinker

Three-prong Sinker

### UV/VIS Detector Wavelength

240 nm

### Flow Cell

10 mm

### Dissolution Medium

Degassed Phosphate Buffer pH 6.5±0.05 with 5M HCl or 5M NaOH solution as required

### Dissolution Medium Vvolume

900 ml

### Paddle Speed

100 rpm

### Bath Temperature

37.0±0.5°

### Sample Time

Every Hour for 24 h

Table 2 provides the in vitro dissolution data of 20 mg Citalopram HBr CR Tablets formulated according to Formulations 1, 2 and 3.

TABLE 2
In vitro Dissolution Data of 20 mg Citalopram HBr CR Tablets
Formulated According to Formulations 1, 2 and 3

| | Formulation 1 | Formulation 2 | Formulation 3 |
|---|---|---|---|
| Time (hrs) | (% dissolved) | (% dissolved) | (% dissolved) |
| 0 | 0 | 0 | 0 |
| 1 | 7.04 | 12.77 | 19.03 |
| 2 | 11.42 | 20.6 | 30.5 |
| 3 | 15.2 | 27.06 | 39.94 |
| 4 | 18.68 | 32.73 | 48.14 |
| 5 | 21.98 | 37.97 | 55.83 |
| 6 | 25.14 | 42.86 | 62.67 |
| 7 | 28.24 | 47.54 | 68.84 |
| 8 | 31.21 | 52.02 | 74.21 |
| 9 | 34.11 | 56.27 | 78.84 |
| 10 | 36.95 | 60.35 | 82.73 |
| 11 | 39.8 | 64.3 | 86.11 |
| 12 | 42.58 | 67.94 | 89.1 |
| 13 | 45.45 | 71.41 | 91.71 |
| 14 | 48.35 | 74.54 | 93.82 |
| 15 | 51.21 | 77.24 | 95.69 |
| 16 | 54.1 | 79.64 | 97.02 |
| 17 | 56.73 | 81.74 | 98.06 |
| 18 | 59.15 | 83.62 | 98.95 |
| 19 | 61.54 | 85.54 | 99.78 |
| 20 | 63.84 | 87.06 | 100.32 |
| 21 | 66.06 | 88.94 | 100.93 |
| 22 | 68.22 | 90.07 | 101.41 |
| 23 | 70.27 | 91.53 | 102.55 |
| 24 | 72.26 | 92.73 | 103.37 |

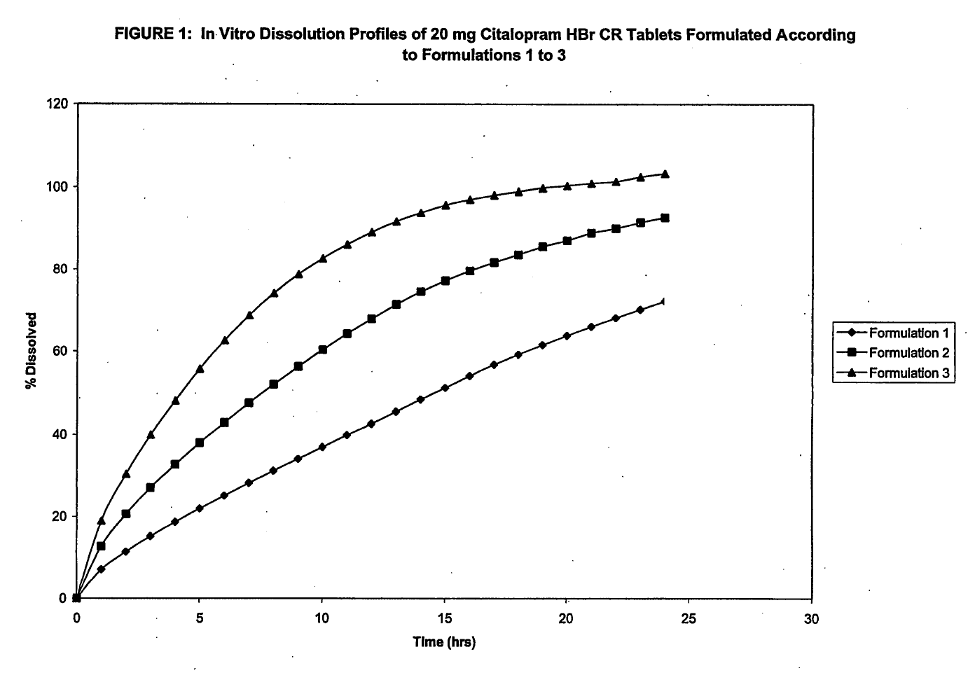

FIG. 1 compares the in vitro dissolution profiles of 20 mg Citalopram HBr CR Tablets formulated according to Formulations 1, 2 and 3. Based on the results of this study, Formulations 1, 2 and 3 were designated as “slow”, “medium” and “fast” formulations, respectively, in comparison to each other.

A pilot, single-dose, open-label, four-way, cross-over study was conducted to evaluate the relative bioavailability of 20 mg Citalopram HBr CR Tablets formulated according to Formulations 1, 2 and 3 versus the reference product, 20 mg CELEXA™ tablets (Lot Number: A304; Expiry Date 04/MR; Lundbeck Canada Inc.), under fasting conditions in normal healthy, non-smoking male volunteers. After a fast of at least 10 hours, each subject received a single dose of one (1) 20 mg Citalopram HBr CR Tablet formulated according to Formulation 1, 2 or 3 with 180 ml of water (Regimens A, B, or C, respectively) or a single dose of one (1) 20 mg CELEXA™ tablet with 180 ml of water (Regimen D). The study periods were separated by a washout period of one (1) week. Blood samples were taken at 0.0 (pre-drug), 1.0, 1.5, 2.0, 2.5, 3.0, 3.5, 4.0, 4.5, 5.0, 5.5, 6.0, 8.0, 10.0, 12.0, 16.0, 24.0, 36.0, 48.0, 72.0, 96.0, 120.0, and 144.0 hours post-drug. Upon completion of the clinical portion of the study, the plasma samples were delivered under frozen conditions to the analytical facility for the analysis of the Citalopram concentrations. Twelve (12) qualified subjects and four (4) alternates were enrolled in the study in two groups. Group I consisted of Subjects #01 through #10, and Group II, consisted of Subjects #11 through #16. Plasma drug concentration, pharmacokinetic and statistical analyses were conducted on eleven (11) subjects.

Table 3 provides the mean plasma Citalopram concentrations (ng/ml) over time after a single dose of one 20 mg Citalopram HBr CR Tablet formulated according to Formulation 1, 2 or 3 or after a single dose of one 20 mg CELEXA™ tablet under fasting conditions.

TABLE 3
Mean Plasma Citalopram Concentrations (n = 11)

| Sample | Formulation 1 | Formulation 2 | Formulation 3 | CELEXA ™ |
|---|---|---|---|---|
| Time | 1 × 20 mg | 1 × 20 mg | 1 × 20 mg | 1 × 20 mg |
| (Hours) | (ng/ml) | (ng/ml) | (ng/ml) | (ng/ml) |
| 0.00 | 0.99 ± 0.97 | 0.86 ± 0.82 | 0.75 ± 0.61 | 0.84 ± 0.78 |
| 1.00 | 1.09 ± 0.95 | 0.93 ± 0.86 | 0.93 ± 0.56 | 4.00 ± 2.86 |
| 1.50 | 1.39 ± 0.95 | 1.20 ± 0.71 | 1.55 ± 0.65 | 10.01 ± 5.01 |
| 2.00 | 1.81 ± 1.13 | 1.80 ± 0.82 | 2.39 ± 0.95 | 14.86 ± 5.18 |
| 2.50 | 2.56 ± 1.55 | 2.44 ± 0.74 | 4.22 ± 1.47 | 18.00 ± 5.98 |
| 3.00 | 3.04 ± 1.52 | 3.17 ± 0.63 | 5.49 ± 1.87 | 20.23 ± 5.22 |
| 3.50 | 3.67 ± 1.68 | 4.28 ± 1.00 | 6.95 ± 2.71 | 20.76 ± 4.13 |
| 4.00 | 4.21 ± 1.60 | 4.99 ± 1.21 | 8.09 ± 2.65 | 21.99 ± 4.10 |
| 4.50 | 4.76 ± 1.78 | 5.55 ± 1.41 | 9.12 ± 2.85 | 21.50 ± 3.39 |
| 5.00 | 5.80 ± 2.24 | 7.17 ± 2.21 | 11.92 ± 3.84 | 21.70 ± 4.50 |
| 5.50 | 6.40 ± 2.37 | 8.50 ± 2.47 | 13.30 ± 2.89 | 22.11 ± 4.12 |
| 6.00 | 7.23 ± 2.88 | 9.10 ± 2.76 | 13.35 ± 2.54 | 20.93 ± 4.57 |
| 8.00 | 8.13 ± 3.00 | 10.16 ± 3.70 | 14.94 ± 3.43 | 21.38 ± 4.74 |
| 10.00 | 9.13 ± 3.11 | 10.56 ± 3.54 | 16.29 ± 3.87 | 19.87 ± 5.37 |
| 12.00 | 9.07 ± 2.79 | 10.52 ± 4.23 | 15.16 ± 3.77 | 18.43 ± 4.17 |
| 16.00 | 8.97 ± 3.51 | 9.78 ± 3.15 | 14.83 ± 4.26 | 15.34 ± 3.04 |
| 24.00 | 8.23 ± 3.35 | 9.40 ± 3.77 | 11.73 ± 3.35 | 12.90 ± 3.36 |
| 36.00 | 7.54 ± 3.92 | 8.39 ± 3.47 | 10.16 ± 3.07 | 10.54 ± 2.29 |
| 48.00 | 6.06 ± 3.08 | 6.00 ± 2.60 | 7.43 ± 1.99 | 8.05 ± 1.73 |
| 72.00 | 4.08 ± 2.23 | 3.63 ± 1.48 | 5.27 ± 2.04 | 5.36 ± 1.82 |
| 96.00 | 2.95 ± 1.60 | 2.86 ± 1.49 | 3.42 ± 1.22 | 3.73 ± 1.57 |
| 120.00 | 1.96 ± 0.93 | 2.09 ± 1.16 | 2.43 ± 1.00 | 2.81 ± 1.30 |
| 144.00 | 1.56 ± 1.09 | 1.34 ± 0.72 | 1.76 ± 0.73 | 2.03 ± 0.92 |

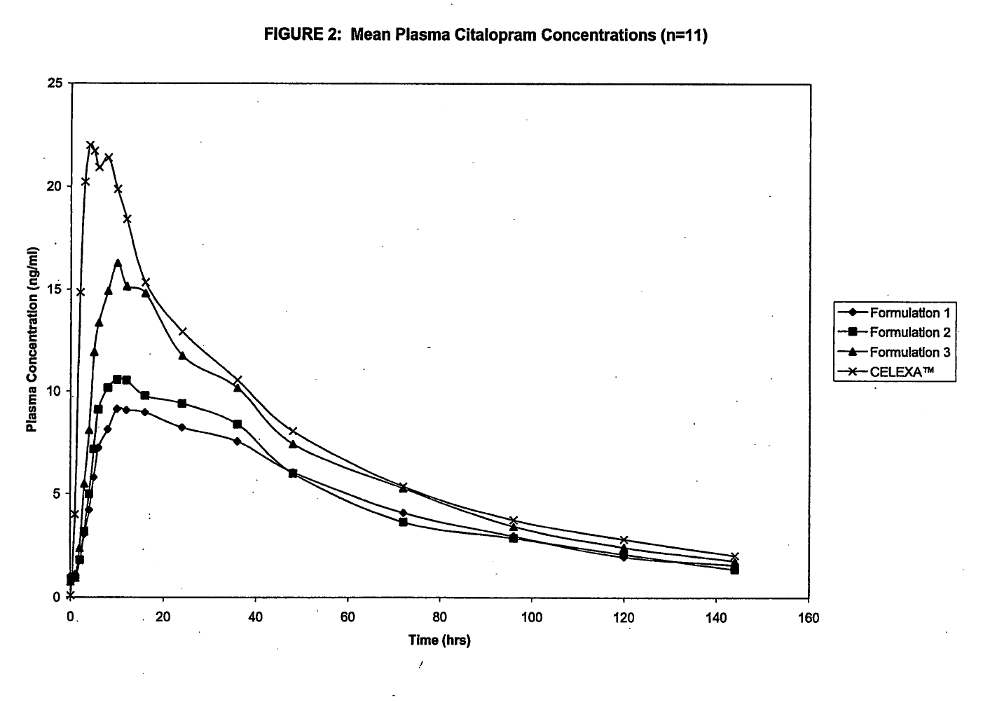

FIG. 2 illustrates the mean plasma Citalopram concentrations (ng/ml) over time after a single dose of one 20 mg Citalopram HBr CR Tablet formulated according to Formulation 1, 2 or 3 or after a single dose of one 20 mg CELEXA™ tablet under fasting conditions.

Table 4 provides the mean pharmacokinetic parameters for plasma Citalopram after a single dose of one 20 mg Citalopram HBr CR Tablet formulated according to Formulation 1, 2 or 3 or after a single dose of one 20 mg CELEXA™ tablet under fasting conditions.

TABLE 4
Mean Pharmacokinetic Parameters for Plasma Citalopram (n = 11)

| | | | | CELEXA ™ |
|---|---|---|---|---|
| | | Formulation 2 | Formulation 3 | (Lot #: A304) |
| | Formulation 1 | 1 × 20 mg | 1 × 20 mg | 1 × 20 mg |
| | 1 × 20 mg | Mean | Mean | Mean |
| Parameter | Mean (% C.V.) | (% C.V.) | (% C.V.) | (% C.V.) |
| AUC_{(O-t)} (ng · hr/ml) | 659.01 (44.39) | 698.17 (40.18) | 907.89 (28.02) | 1059.27 (23.61) |
| AUC_{(O-inf)} | 688.21 (37.66) | 790.02 (42.31) | 1026.34 (29.61) | 1150.03 (24.69) |
| (ng · hr/ml) | | | | |
| Cₘₐₓ (ng/ml) | 10.36 (31.23) | 11.82 (30.41) | 17.21 (18.89) | 24.02 (17.48) |
| Tₘₐₓ (hours) | 19.27 (66.65) | 16.55 (55.92) | 9.14 (33.39) | 5.87 (40.34) |
| t_{1/2} (hours) | 48.71 (19.19) | 45.54 (13.92) | 45.37 (14.07) | 46.01 (9.44) |
| Kₑₗ (hour⁻¹) | 0.015 (19.930) | 0.015 (14.097) | 0.016 (14.627) | 0.015 (9.741) |

In vitro dissolution studies were conducted on 20 mg Citalopram HBr CR Tablets formulated according to Formulations 2 and 4. Table 5 provides the in vitro dissolution data of 20 mg Citalopram HBr CR Tablets formulated according to Formulations 2 and 4.

TABLE 5
In vitro Dissolution Data of 20 mg Citalopram HBr CR Tablets
Formulated According to Formulations 2 and 4

| | Formulation 2 | Formulation 4 |
|---|---|---|
| Time (hrs) | (% dissolved) | (% dissolved) |
| 0 | 0 | 0 |
| 1 | 12.77 | 3.36 |
| 2 | 20.6 | 7.98 |
| 3 | 27.06 | 12.75 |
| 4 | 32.73 | 17.65 |
| 5 | 37.97 | 22.73 |
| 6 | 42.86 | 27.92 |
| 7 | 47.54 | 32.95 |
| 8 | 52.02 | 37.89 |
| 9 | 56.27 | 42.61 |
| 10 | 60.35 | 47.14 |
| 11 | 64.3 | 51.4 |
| 12 | 67.94 | 55.53 |
| 13 | 71.41 | 59.39 |
| 14 | 74.54 | 63.08 |
| 15 | 77.24 | 66.53 |
| 16 | 79.64 | 69.8 |
| 17 | 81.74 | 72.79 |
| 18 | 83.62 | 75.61 |
| 19 | 85.54 | 78.22 |
| 20 | 87.06 | 80.67 |
| 21 | 88.94 | 82.98 |
| 22 | 90.07 | 85.05 |
| 23 | 91.53 | 86.96 |
| 24 | 92.73 | 88.78 |

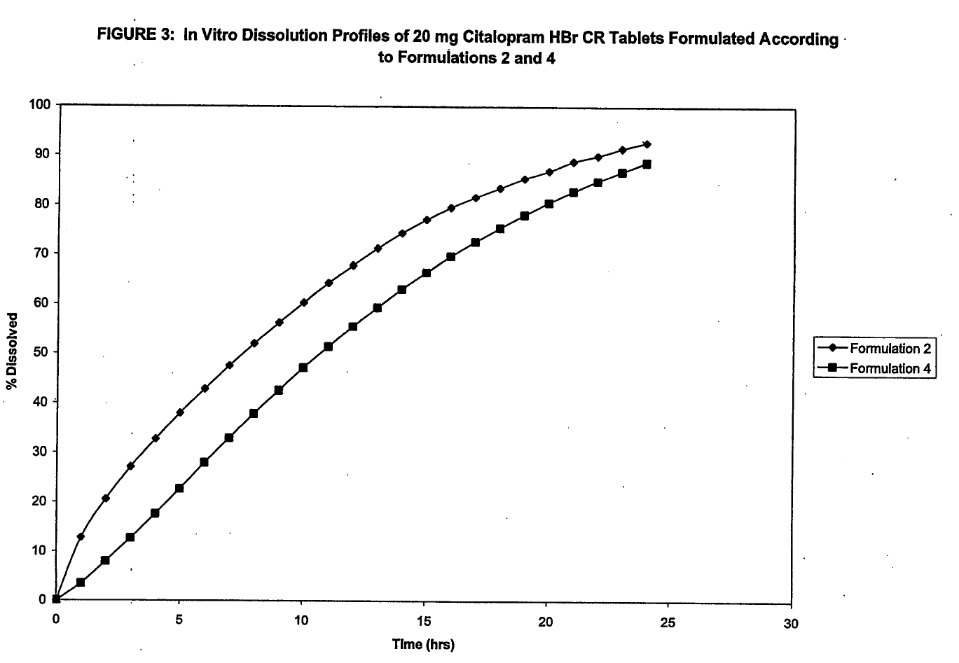

FIG. 3 compares the in vitro dissolution profiles of 20 mg Citalopram HBr CR Tablets formulated according to Formulations 2 and 4.

A pilot, randomized, single-dose, open-label, four-way, cross-over study was conducted to evaluate the relative bioavailability of 20 mg Citalopram HBr 20 mg CR Tablets formulated according to Formulations 2 and 4 under fasted and fed conditions in normal healthy, non-smoking male volunteers. After a fast of at least 10 hours (for Regimens A and C), or five (5) minutes after complete ingestion of a high-fat content breakfast (for Regimens B and D), each subject received a single dose of one (1) 20 mg Citalopram CR Tablet formulated according to Formulation 4 (fasted or fed) with 180 ml of water or a single dose of one (1) 20 mg Citalopram CR Tablet formulated according to Formulation 2 (fasted or fed) with 180 ml of water. There was a washout period of one (1) week between study periods. Following each drug administration, blood samples were collected at 0.0 (pre-drug), 1.0, 1.5, 2.0, 2.5, 3.0, 3.5, 4.0, 4.5, 5.0, 5.5, 6.0, 8.0, 10.0, 12.0, 16.0, 24.0, 36.0, 48.0, 72.0, 96.0, 100.0, and 144.0 hours post-drug. Upon completion of the clinical portion of the study, plasma samples were delivered under frozen conditions to the analytical facility for the analysis of Citalopram concentrations. Twelve (12) subjects and three (3) alternates were entered into the study. Plasma drug concentration, pharmacokinetic and statistical analyses were conducted on the twelve (12) subjects.

Table 6 provides the mean plasma Citalopram concentrations (ng/ml) over time after a single dose of one 20 mg Citalopram HBr CR Tablet formulated according to Formulation 4 or 2 under fasting or fed conditions.

TABLE 6
Mean Plasma Citalopram Concentrations (n = 12)

| | Formulation | Formulation 4 | Formulation 2 | Formulation 2 |
|---|---|---|---|---|
| Sample | 4 (Fasted) | (Fed) | (Fasted) | (Fed) |
| Time | 1 × 20 mg | 1 × 20 mg | 1 × 20 mg | 1 × 20 mg |
| (Hours) | (ng/ml) | (ng/ml) | (ng/ml) | (ng/ml) |
| 0.00 | 0.57 ± 0.58 | 0.49 ± 0.53 | 0.68 ± 0.64 | 0.70 ± 0.66 |
| 1.00 | 0.58 ± 0.57 | 0.54 ± 0.48 | 0.81 ± 0.65 | 0.95 ± 0.84 |
| 1.50 | 0.68 ± 0.61 | 0.58 ± 0.50 | 1.05 ± 0.72 | 1.51 ± 1.17 |
| 2.00 | 0.90 ± 0.55 | 0.75 ± 0.44 | 1.40 ± 0.84 | 2.53 ± 1.65 |
| 2.50 | 1.15 ± 0.67 | 0.97 ± 0.42 | 1.99 ± 0.99 | 3.83 ± 2.74 |
| 3.00 | 1.49 ± 0.74 | 1.29 ± 0.48 | 2.56 ± 1.21 | 4.19 ± 2.69 |
| 3.50 | 1.91 ± 0.79 | 1.57 ± 0.47 | 3.13 ± 1.42 | 5.12 ± 2.97 |
| 4.00 | 2.57 ± 0.86 | 2.03 ± 0.53 | 3.78 ± 1.59 | 6.04 ± 3.45 |
| 4.50 | 3.11 ± 1.12 | 2.39 ± 0.71 | 4.43 ± 1.96 | 6.51 ± 2.83 |
| 5.00 | 4.18 ± 1.42 | 3.61 ± 1.31 | 5.17 ± 2.28 | 8.56 ± 4.41 |
| 5.50 | 5.30 ± 1.76 | 4.65 ± 1.65 | 6.26 ± 3.01 | 9.43 ± 5.67 |
| 6.00 | 5.98 ± 1.72 | 5.42 ± 1.77 | 7.47 ± 3.19 | 10.51 ± 4.95 |
| 8.00 | 7.28 ± 2.08 | 7.54 ± 2.24 | 8.16 ± 3.08 | 11.01 ± 3.37 |
| 10.00 | 8.09 ± 2.50 | 9.15 ± 2.47 | 9.25 ± 3.77 | 12.34 ± 3.54 |
| 12.00 | 8.20 ± 2.85 | 9.95 ± 2.30 | 9.39 ± 4.18 | 12.23 ± 3.30 |
| 16.00 | 8.74 ± 4.03 | 11.58 ± 2.80 | 9.68 ± 4.58 | 13.29 ± 2.86 |
| 24.00 | 7.24 ± 3.22 | 10.68 ± 3.23 | 7.59 ± 3.49 | 11.38 ± 2.91 |
| 36.00 | 6.32 ± 3.22 | 9.30 ± 2.93 | 6.48 ± 3.15 | 9.27 ± 1.85 |
| 48.00 | 4.81 ± 2.68 | 7.14 ± 2.14 | 5.04 ± 2.62 | 7.00 ± 2.16 |
| 72.00 | 2.83 ± 1.61 | 4.43 ± 1.41 | 3.19 ± 1.80 | 4.45 ± 1.19 |
| 96.00 | 1.96 ± 1.15 | 2.95 ± 1.13 | 2.27 ± 1.47 | 2.96 ± 0.92 |
| 120.00 | 1.34 ± 0.93 | 2.10 ± 0.84 | 1.48 ± 1.07 | 2.02 ± 0.69 |
| 144.00 | 0.96 ± 0.71 | 1.42 ± 0.64 | 1.07 ± 0.79 | 1.38 ± 0.62 |

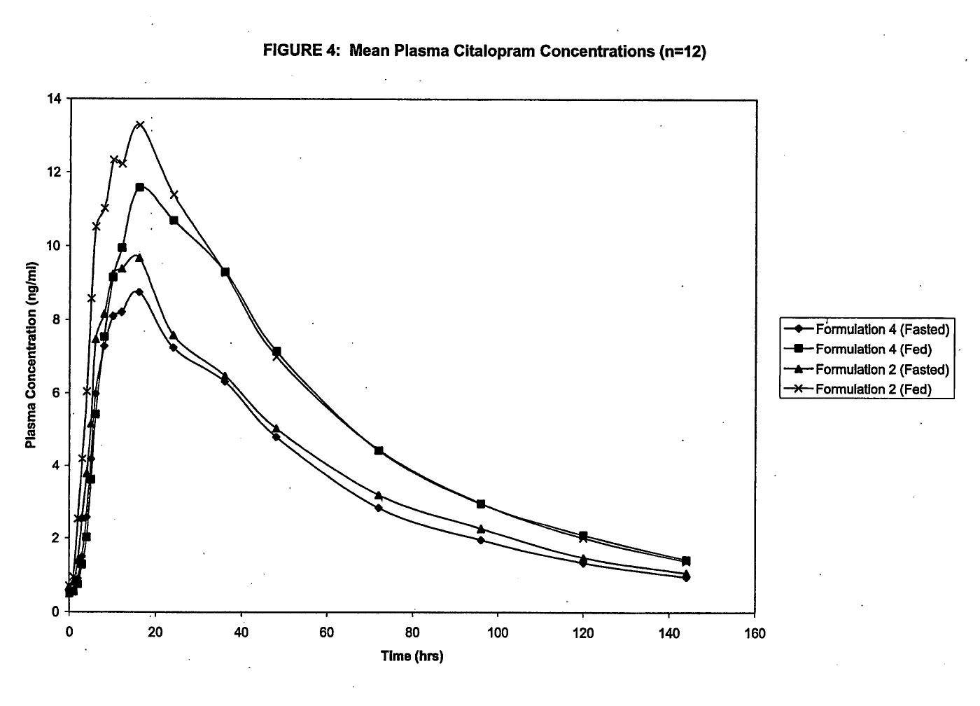

FIG. 4 illustrates the mean plasma Citalopram concentrations (ng/ml) over time after a single dose of one 20 mg Citalopram HBr CR Tablet formulated according to Formulation 4 or 2 under fasting or fed conditions.

Table 7 provides the mean pharmacokinetic parameters for plasma Citalopram after a single dose of one 20 mg Citalopram HBr CR Tablet formulated according to Formulation 4 or 2 under fasting or fed conditions.

TABLE 7
Mean Pharmacokinetic Parameters for Plasma Citalopram (n = 12)

| | Formulation 4 | Formulation 4 | Formulation 2 | Formulation 2 |
|---|---|---|---|---|
| | (Fasted) | (Fed) | (Fasted) | (Fed) |
| | 1 × 20 mg | 1 × 20 mg | 1 × 20 mg | 1 × 20 mg |
| Parameter | Mean (% C.V.) | Mean (% C.V.) | Mean (% C.V.) | Mean (% C.V.) |
| AUC_{(O-t)} | 522.63 (49.28) | 740.52 (26.33) | 569.46 (49.87) | 795.15 (24.02) |
| (ng · hr/ml) | | | | |
| AUC_{(O-inf)} | 591.05 (53.90) | 834.52 (28.88) | 643.50 (53.64) | 891.41 (27.09) |
| (ng · hr/ml) | | | | |
| Cₘₐₓ (ng/ml) | 9.46 (37.17) | 12.83 (21.82) | 10.49 (43.06) | 14.61 (29.78) |
| Tₘₐₓ (hours) | 13.67 (33.51) | 18.83 (37.47) | 12.84 (30.08) | 15.96 (31.28) |
| t_{1/2} (hours) | 43.66 (22.30) | 44.28 (19.61) | 42.74 (20.77) | 43.93 (28.35) |
| Kₑₗ (hour⁻¹) | 0.017 (19.294) | 0.016 (19.92) | 0.017 (21.014) | 0.017 (27.221) |

### Example 2

Table 8 provides the composition of three Citalopram HBr CR Tablet formulations (Formulations 5, 6 and 7).

TABLE 8
Citalopram HBr CR Tablet Formulations

| | Formulation | Formulation | Formulation |
|---|---|---|---|
| Ingredients | 5 (% w/w) | 6 (% w/w) | 7 (% w/w) |
| Citalopram HBr | 6.25 | 6.25 | 6.18 |
| HPMC (METHOCEL ® | 35.00 | 35.00 | 34.59 |
| K4MPrem CR) | | | |
| Lactose Anhydrous (DT) | 48.00 | 48.00 | 47.43 |
| MCC (AVICEL ® PH 101) | 8.00 | 8.00 | 7.90 |
| PVP (KOLLIDON ® 29/32) | 1.75 | 1.75 | 1.93 |
| Magnesium Stearate | 1.00 | NA | 0.99 |
| Stearic Acid | NA | 1.00 | NA |
| Carbomer 941 | NA | NA | 0.99 |
| (CARBOPOL ® 971P) | | | |

As can be seen from Table 8, Formulation 5 comprises magnesium stearate, Formulation 6 comprises stearic acid, and Formulation 7 comprises Carbomer 941.

In vitro dissolution studies were conducted on 20 mg Citalopram HBr CR Tablets formulated according to Formulations 5, 6 and 7. Table 9 provides the in vitro dissolution data of 20 mg Citalopram HBr CR Tablets formulated according to Formulations 5, 6 and 7.

TABLE 9
In vitro Dissolution Data of 20 mg Citalopram HBr CR Tablets
Formulated According to Formulations 5, 6 and 7

| | Formulation 5 | Formulation 6 | Formulation 7 |
|---|---|---|---|
| Time (hrs) | (% dissolved) | (% dissolved) | (% dissolved) |
| 0 | 0 | 0 | 0 |
| 1 | 19.07 | 18.11 | 14.71 |
| 2 | 30.51 | 29.38 | 23.75 |
| 3 | 39.96 | 38.1 | 31.5 |
| 4 | 48.29 | 45.97 | 38.49 |
| 5 | 55.8 | 53.04 | 44.87 |
| 6 | 62.52 | 59.42 | 50.8 |
| 7 | 68.46 | 65.04 | 56.1 |
| 8 | 73.74 | 70.3 | 61.04 |
| 9 | 78.54 | 74.89 | 65.43 |
| 10 | 82.9 | 78.92 | 69.49 |
| 11 | 86.64 | 82.41 | 73.13 |
| 12 | 89.63 | 85.54 | 76.53 |
| 13 | 92.04 | 88.13 | 79.61 |
| 14 | 94.05 | 90.34 | 82.33 |
| 15 | 95.65 | 92.23 | 84.83 |
| 16 | 97.1 | 93.62 | 87.06 |
| 17 | 97.86 | 94.75 | 89.11 |
| 18 | 98.7 | 96 | 90.97 |
| 19 | 99.46 | 96.85 | 92.71 |
| 20 | 99.73 | 97.42 | 94.45 |
| 21 | 99.87 | 97.68 | 95.84 |
| 22 | 100.06 | 98.42 | 97.23 |
| 23 | 100.3 | 99.01 | 98.57 |
| 24 | 100.35 | 100.15 | 99.66 |

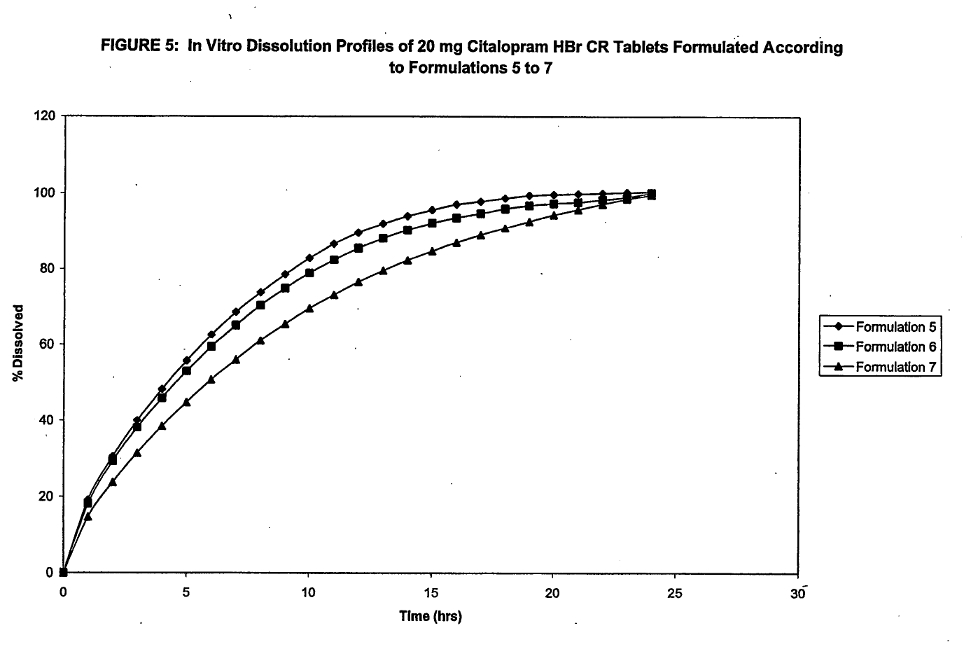

FIG. 5 compares the in vitro dissolution profiles of 20 mg Citalopram HBr CR Tablets formulated according to Formulations 5, 6 and 7.

### Examples 3 to 5

Various further Citalopram HBr CR Tablet formulations were made to test the influence of polymer concentration; the use of surfactant; the use of L-Tartaric acid to improve solubility and absorption; and the use of polyvinyl pyrrolidone (PVP) to improve solubility. Each of these tests is outlined below, with accompanying dissolution data.

### Example 3

Tables 10A and 10B provide the composition of ten Citalopram HBr CR Tablet formulations comprising different concentrations of the polymer hydroxypropyl methylcellulose (METHOCEL® K4M Premium CR) formulated to test the use of polymer and to ascertain the influence of polymer concentration on the in vitro dissolution profiles of the formulations.

TABLE 10A
Citalopram HBr CR Tablet Formulations To Test The Influence
Of Polymer Concentration

| | Formulation No. | | | | |
|---|---|---|---|---|---|
| | 5 | 8 | | 10 | 11 |
| | (% | (% | 9 | (% | (% |
| Ingredient | w/w) | w/w) | (% w/w) | w/w) | w/w) |
| Citalopram HBr | 6.25 | 6.25 | 6.25 | 6.25 | 6.25 |
| HPMC (METHOCEL ® | 35.00 | 30.00 | 20.00 | 15.00 | 10.00 |
| K4MPrem CR) | | | | | |
| Lactose Anhydrous (DT) | 48.00 | 53.00 | 63.00 | 68.00 | 73.00 |
| MCC (AVICEL ® PH 101) | 8.00 | 8.00 | 8.00 | 8.00 | 8.00 |
| PVP (KOLLIDON ® 29/32) | 1.75 | 1.75 | 1.75 | 1.75 | 1.75 |
| Magnesium Stearate | 1.00 | 1.00 | 1.00 | 1.00 | 1.00 |

TABLE 10B
Citalopram HBr CR Tablet Formulations To Test The Influence
Of Polymer Concentration

| | Formulation No. | | | | |
|---|---|---|---|---|---|
| | 12 | | | | 16 |
| | (% | 13 | 14 | 15 | (% |
| Ingredient | w/w) | (% w/w) | (% w/w) | (% w/w) | w/w) |
| Citalopram HBr | 6.25 | 6.25 | 6.25 | 6.25 | 6.25 |
| HPMC | 20.00 | 22.00 | 30.00 | 26.00 | 28.00 |
| (METHOCEL ® | | | | | |
| K4MPrem CR) | | | | | |
| Lactose Anhydrous | 63.00 | 61.00 | 53.00 | 57.00 | 55.00 |
| (DT) | | | | | |
| MCC (AVICEL ® PH | 8.00 | 8.00 | 8.00 | 8.00 | 8.00 |
| 101) | | | | | |
| PVP (KOLLIDON ® | 1.75 | 1.75 | 1.75 | 1.75 | 1.75 |
| 29/32) | | | | | |
| Magnesium Stearate | 1.00 | 1.00 | 1.00 | 1.00 | 1.00 |

In vitro dissolution studies were conducted on 20 mg Citalopram HBr CR Tablets formulated according to Formulations 5 and 8 to 16. Tables 11A and 11B provide the in vitro dissolution data of 20 mg Citalopram HBr CR Tablets formulated according to Formulations 8 to 16.

TABLE 11A
In vitro Dissolution Data of 20 mg Citalopram HBr CR Tablets
Formulated According to Formulations 8 to 11

| Time | Formulation 8 | Formulation 9 | Formulation 10 | Formulation 11 |
|---|---|---|---|---|
| (hrs) | (% dissolved) | (% dissolved) | (% dissolved) | (% dissolved) |
| 0 | 0 | 0 | 0 | 0 |
| 1 | 19 | 20 | 25 | 29 |
| 2 | 30 | 32 | 38 | 42 |
| 3 | 40 | 42 | 50 | 58 |
| 4 | 47 | 50 | 59 | 76 |
| 5 | 54 | 58 | 68 | 94 |
| 6 | 60 | 65 | 76 | 95 |
| 7 | 66 | 71 | 82 | 95 |
| 8 | 71 | 77 | 87 | 96 |
| 9 | 75 | 81 | 93 | 96 |
| 10 | 78 | 85 | 96 | 96 |
| 11 | 82 | 88 | 98 | 96 |
| 12 | 84 | 91 | 99 | 97 |
| 13 | 87 | 93 | 100 | 97 |
| 14 | 88 | 95 | 100 | 97 |
| 15 | 90 | 96 | 100 | |
| 16 | 91 | 96 | 100 | |
| 17 | 92 | 97 | 100 | |
| 18 | 93 | 97 | 100 | |
| 19 | 94 | 98 | | |
| 20 | 94 | 99 | | |
| 21 | 95 | 99 | | |
| 22 | 95 | 98 | | |
| 23 | 96 | 98 | | |
| 24 | 96 | 99 | | |

TABLE 11B
In vitro Dissolution Data of 20 mg Citalopram HBr CR Tablets
Formulated According to Formulations 12 to 16

| | Formulation | Formulation | Formulation | Formulation | Formulation |
|---|---|---|---|---|---|
| Time | 12 | 13 | 14 | 15 | 16 |
| (hrs) | (% dissolved) | (% dissolved) | (% dissolved) | (% dissolved) | (% dissolved) |
| 0 | 0 | 0 | 0 | 0 | 0 |
| 1 | 23 | 23 | 20 | 22 | 21 |
| 2 | 36 | 36 | 32 | 35 | 33 |
| 3 | 47 | 47 | 42 | 45 | 43 |
| 4 | 56 | 56 | 51 | 55 | 51 |
| 5 | 64 | 64 | 59 | 63 | 59 |
| 6 | 72 | 71 | 65 | 69 | 66 |
| 7 | 83 | 78 | 71 | 75 | 72 |
| 8 | 87 | 83 | 76 | 80 | 77 |
| 9 | 91 | 87 | 80 | 86 | 81 |
| 10 | | 91 | 84 | 88 | 84 |
| 11 | | 94 | 87 | 91 | 87 |
| 12 | | 97 | 90 | 93 | 90 |
| 13 | | 99 | 92 | 95 | 92 |
| 14 | | 100 | 94 | 98 | 94 |
| 15 | | 101 | 96 | 99 | 96 |
| 16 | | 102 | 97 | 100 | 97 |
| 17 | | 103 | 98 | 101 | 98 |
| 18 | | | 99 | 102 | 98 |
| 19 | | | 100 | 102 | 98 |

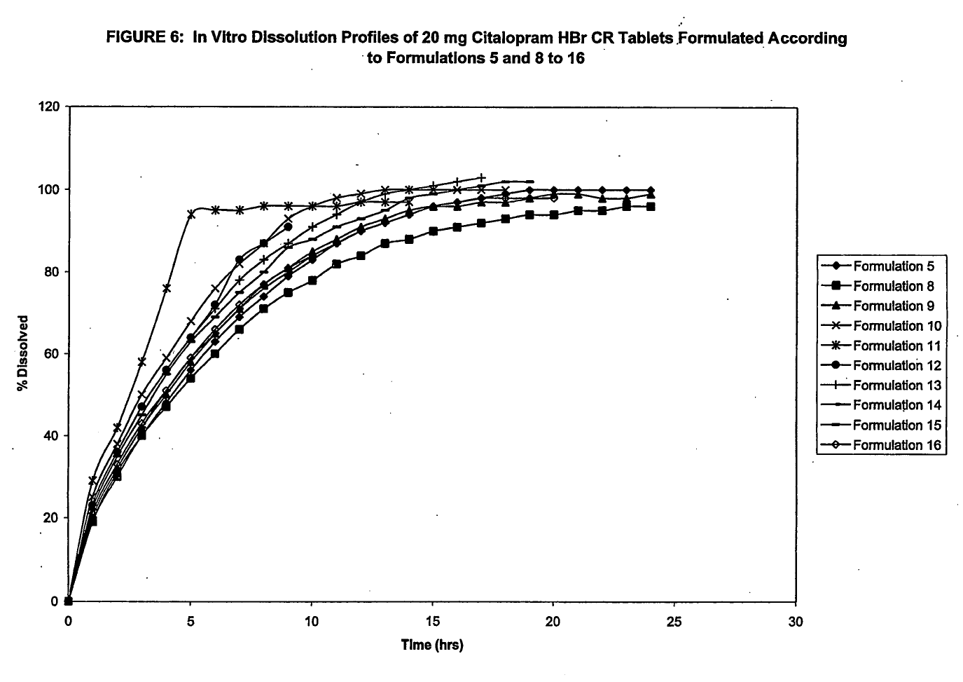

FIG. 6 compares the in vitro dissolution profiles of 20 mg Citalopram HBr CR Tablets formulated according to Formulations 5 and 8 to 16.

### Example 4

Tables 12A, 12B and 12C provide the composition of sixteen Citalopram HBr CR Tablet formulations comprising different surfactants formulated to test the use of surfactant and to ascertain its effect on the in vitro dissolution profiles of the formulations.

TABLE 12A
Citalopram HBr CR Tablet Formulations To Test The Use of Surfactant

| | Formulation No. | | | | | |
|---|---|---|---|---|---|---|
| | 17 | 18 | 19 | 20 | 21 | 22 |
| Ingredient | (% w/w) | (% w/w) | (% w/w) | (% w/w) | (% w/w) | (% w/w) |
| Citalopram HBr | 6.25 | 6.25 | 6.25 | 6.25 | 7.14 | 7.14 |
| HPMC (METHOCEL ® | 35.00 | 35.00 | 30.00 | 30.00 | 30.00 | 30.00 |
| K4MPrem CR) | | | | | | |
| Lactose Anhydrous (DT) | 46.00 | 46.00 | 48.00 | 48.00 | 47.00 | 47.00 |
| MCC (AVICEL ® PH 101) | 8.00 | 8.00 | 8.00 | 8.00 | 8.00 | 8.00 |
| PVP (KOLLIDON ® 29/32) | 1.75 | 1.75 | 1.75 | NA | 2.00 | 2.00 |
| PVP (KOLLIDON ® 90F) | NA | NA | NA | 1.75 | NA | NA |
| Magnesium Stearate | 1.00 | 1.00 | 1.00 | 1.00 | 1.00 | 1.00 |
| Bile Salt | 2.00 | NA | NA | NA | NA | NA |
| SLS | NA | 2.00 | NA | NA | NA | NA |
| Poloxamer 407 | NA | NA | 5.00 | NA | 5.00 | NA |
| (LUTROL ®-F 127) | | | | | | |
| Poloxamer 188 | NA | NA | NA | 5.00 | NA | NA |
| (LUTROL ®-F 68) | | | | | | |
| PEG-40 hydrogenated castor | NA | NA | NA | NA | NA | 5.00 |
| oil (CREMOPHOR ® RH 40) | | | | | | |

TABLE 12B
Citalopram HBr CR Tablet Formulations To Test The Use of Surfactant

| | Formulation No. | | | | | |
|---|---|---|---|---|---|---|
| | 23 | 24 | 25 | 26 | 27 | 28 |
| Ingredient | (% w/w) | (% w/w) | (% w/w) | (% w/w) | (% w/w) | (% w/w) |
| Citalopram HBr | 7.14 | 7.14 | 7.14 | 7.14 | 7.14 | 71.4 |
| HPMC (METHOCEL ® K4MPrem | 30.00 | 30.00 | 30.00 | 30.00 | 30.00 | 30.00 |
| CR) | | | | | | |
| Lactose Anhydrous (DT) | 47.00 | 47.00 | 47.00 | 47.00 | 47.00 | 47.00 |
| MCC (AVICEL ® PH 101) | 8.00 | 8.00 | 8.00 | 8.00 | 8.00 | 8.00 |
| PVP (KOLLIDON ® 90F) | 2.00 | 2.00 | 2.00 | 2.00 | 2.00 | 2.00 |
| Magnesium Stearate | 1.00 | 1.00 | 1.00 | 1.00 | 1.00 | 1.00 |
| PEG-3350 | 5.00 | NA | NA | NA | NA | NA |
| PEG-600 | NA | 5.00 | NA | NA | NA | NA |
| PEG-8000 | NA | NA | 5.00 | NA | NA | NA |
| Saturated polyglycolized | NA | NA | NA | 5.00 | NA | NA |
| glycerides from hydrogenated | | | | | | |
| vegetable oils (GELUCIRE ® | | | | | | |
| 44/14) | | | | | | |
| Saturated polyglycolized | NA | NA | NA | NA | 5.00 | NA |
| glycerides (GELUCIRE ® 50/13) | | | | | | |
| Vitamin E TPGS | NA | NA | NA | NA | NA | 5.00 |

TABLE 12C
Citalopram HBr CR Tablet Formulations To Test The Use of
Surfactant

| | Formulation No. | | | |
|---|---|---|---|---|
| | 29 | 30 | 31 | 32 |
| Ingredient | (% w/w) | (% w/w) | (% w/w) | (% w/w) |
| Citalopram HBr | 7.14 | 7.14 | 7.14 | 7.14 |
| HPMC (METHOCEL ® | 30.00 | 30.00 | 30.00 | 30.00 |
| K4MPrem CR) | | | | |
| Lactose Anhydrous (DT) | 47.00 | 47.00 | 47.00 | 47.00 |
| MCC (AVICEL ® PH | 8.00 | 8.00 | 8.00 | 8.00 |
| 101) | | | | |
| PVP (KOLLIDON ® | 2.00 | 2.00 | 2.00 | 2.00 |
| 90F) | | | | |
| Magnesium Stearate | 1.00 | 1.00 | 1.00 | 1.00 |
| LUTROL ® E 1500 | 5.00 | NA | NA | NA |
| Sucrose stearate | NA | 5.00 | NA | NA |
| (CRODESTA ®-F 160) | | | | |
| Mannitol | NA | NA | 5.00 | NA |
| CAPMUL ® PG8 | NA | NA | NA | 5.00 |

In vitro dissolution studies were conducted on 20 mg Citalopram HBr CR Tablets formulated according to Formulations 17- to 32. Tables 13A, 13B and 13C provide the in vitro dissolution data of 20 mg Citalopram HBr CR Tablets formulated according to Formulations 17 to 32.

TABLE 13A
In vitro Dissolution Data of 20 mg Citalopram HBr CR Tablets
Formulated According to Formulations 17 to 22

| | Formulation | Formulation | Formulation | Formulation | Formulation | Formulation |
|---|---|---|---|---|---|---|
| | 17 | 18 | 19 | 20 | 21 | 22 |
| Time | (% | (% | (% | (% | (% | (% |
| (hrs) | dissolved) | dissolved) | dissolved) | dissolved) | dissolved) | dissolved) |
| 0 | 0 | 0 | 0 | 0 | 0 | 0 |
| 1 | 15 | 11 | 20 | 18 | 21 | 19 |
| 2 | 24 | 18 | 32 | 28 | 33 | 31 |
| 3 | 31 | 24 | 42 | 36 | 43 | 40 |
| 4 | 38 | 30 | 51 | 43 | 52 | 48 |
| 5 | 44 | 35 | 58 | 49 | 59 | 55 |
| 6 | 50 | 40 | 65 | 55 | 65 | 62 |
| 7 | 55 | 45 | 71 | 61 | 71 | 67 |
| 8 | 59 | 50 | 76 | 66 | 76 | 73 |
| 9 | 64 | 54 | 81 | 71 | 80 | 82 |
| 10 | 68 | 58 | 85 | 75 | 84 | |
| 11 | 71 | 61 | 89 | 79 | 87 | |
| 12 | 75 | 64 | 92 | 82 | 90 | |
| 13 | 78 | 67 | 94 | 86 | 92 | |
| 14 | 80 | 70 | 96 | 88 | 94 | |
| 15 | 83 | 73 | 98 | 91 | 96 | |
| 16 | 85 | 75 | 99 | 92 | 97 | |
| 17 | 86 | 78 | 100 | 94 | 98 | |
| 18 | 88 | 80 | 101 | 95 | 99 | |
| 19 | 89 | 82 | 102 | 97 | 100 | |
| 20 | 91 | 83 | 102 | 98 | 100 | |
| 21 | 92 | 85 | 102 | 98 | 101 | |
| 22 | 93 | 87 | 102 | 99 | 101 | |
| 23 | 94 | 88 | 103 | 99 | 101 | |
| 24 | 95 | 89 | | 100 | 102 | |

TABLE 13B
In vitro Dissolution Data of 20 mg Citalopram HBr CR Tablets
Formulated According to Formulations 23 to 28

| | Formulation | Formulation | Formulation | Formulation | Formulation | Formulation |
|---|---|---|---|---|---|---|
| | 23 | 24 | 25 | 26 | 27 | 28 |
| Time | (% | (% | (% | (% | (% | (% |
| (hrs) | dissolved) | dissolved) | dissolved) | dissolved) | dissolved) | dissolved) |
| 0 | 0 | 0 | 0 | 0 | 0 | 0 |
| 1 | 21 | 21 | 20 | 21 | 19 | 19 |
| 2 | 32 | 33 | 31 | 32 | 30 | 29 |
| 3 | 42 | 44 | 41 | 41 | 40 | 38 |
| 4 | 51 | 53 | 50 | 50 | 48 | 46 |
| 5 | 59 | 60 | 57 | | 55 | 53 |
| 6 | 66 | 67 | 64 | | 62 | 59 |
| 7 | 72 | 73 | 71 | | 68 | 63 |
| 8 | 78 | 79 | 76 | | 74 | 69 |
| 9 | 83 | 83 | 81 | | 78 | 73 |
| 10 | 87 | 87 | 85 | | 83 | 77 |
| 11 | 90 | 90 | 88 | | 86 | 81 |
| 12 | 93 | 92 | 91 | | 89 | 84 |
| 13 | 95 | 94 | 93 | | 92 | 87 |
| 14 | 97 | 96 | 95 | | 94 | 90 |
| 15 | 99 | 97 | 97 | | 96 | 92 |
| 16 | 100 | 98 | 98 | | 98 | 94 |
| 17 | 101 | 99 | 99 | | 100 | 96 |
| 18 | 102 | 100 | 100 | | 102 | 98 |
| 19 | 102 | 100 | 100 | | 104 | 99 |
| 20 | 103 | | 100 | | | |

TABLE 13C
In vitro Dissolution Data of 20 mg Citalopram HBr CR Tablets
Formulated According to Formulations 29 to 32

| | | Formulation | Formulation | Formulation |
|---|---|---|---|---|
| Time | Formulation 29 | 30 | 31 | 32 |
| (hrs) | (% dissolved) | (% dissolved) | (% dissolved) | (% dissolved) |
| 0 | 0 | 0 | 0 | 0 |
| 1 | 21 | 17 | 21 | 21 |
| 2 | 33 | 27 | 33 | 32 |
| 3 | 43 | 36 | 43 | 42 |
| 4 | 52 | 43 | 52 | 51 |
| 5 | 60 | 50 | 60 | 59 |
| 6 | 68 | 56 | 66 | 66 |
| 7 | 74 | 61 | 72 | 72 |
| 8 | 80 | 66 | 77 | 77 |
| 9 | 84 | 70 | 82 | 82 |
| 10 | 88 | | 85 | 86 |
| 11 | 91 | | 89 | 89 |
| 12 | 94 | | 91 | 91 |
| 13 | 96 | | 94 | 93 |
| 14 | 97 | | 95 | 95 |
| 15 | 98 | | 97 | 96 |
| 16 | 99 | | 98 | 98 |
| 17 | 100 | | 98 | 99 |
| 18 | 101 | | 99 | 100 |
| 19 | 101 | | 99 | 101 |
| 20 | 101 | | 100 | 102 |

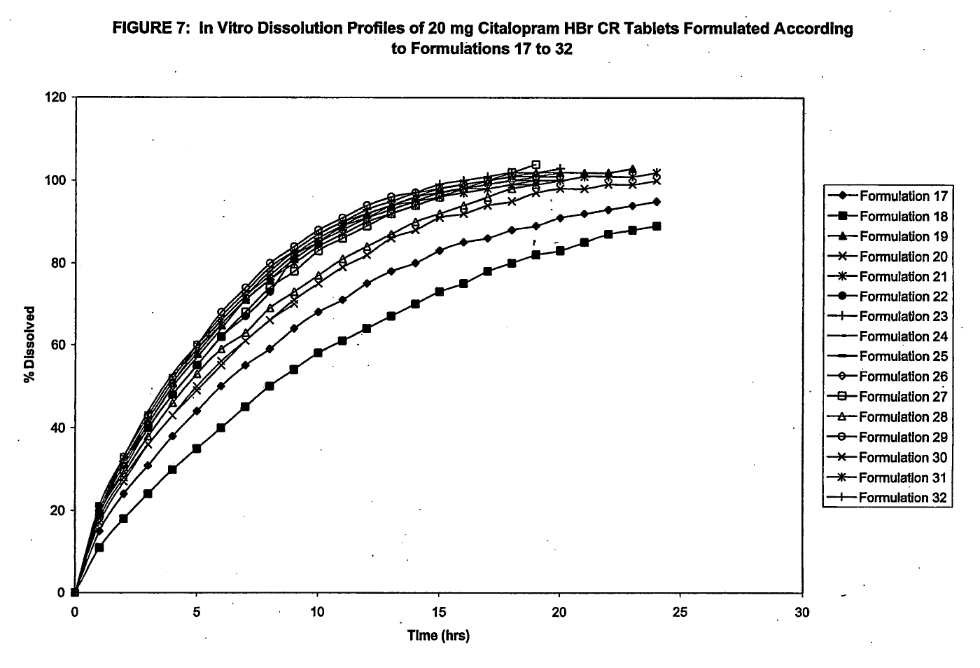

FIG. 7 compares the in vitro dissolution profiles of 20 mg Citalopram HBr CR Tablets formulated according to Formulations 17 to 32.

### Example 5

Table 14 provides the composition of a Citalopram HBr CR Tablet formulation comprising L-Tartaric add formulated to test the use of L-Tartaric acid and to ascertain its effect on the in vitro dissolution profile of the formulation.

TABLE 14
Citalopram HBr CR Tablet Formulation to Test the Use of L-
Tartaric Acid

| | Formulation 33 |
|---|---|
| Ingredient | (% w/w) |
| Citalopram HBr | 6.25 |
| HPMC (METHOCEL ® K4MPrem CR) | 35.00 |
| Lactose Anhydrous (DT) | 43.00 |
| MCC (AVICEL ® PH 101) | 8.00 |
| PVP (KOLLIDON ® 29/32) | 1.75 |
| Magnesium Stearate | 1.00 |
| L-Tartaric Acid | 5 |

In vitro dissolution studies were conducted on 20 mg Citalopram HBr CR Tablets formulated according to Formulations 33. Table 15 provides the in vitro dissolution data of 20 mg Citalopram HBr CR Tablets formulated according to Formulation 33.

TABLE 15
In vitro Dissolution Data of 20 mg Citalopram HBr CR Tablets
Formulated According to Formulation 33

| | Formulation 33 |
|---|---|
| Time (hrs) | (% dissolved) |
| 0 | 0 |
| 1 | 21 |
| 2 | 33 |
| 3 | 43 |
| 4 | 52 |
| 5 | 59 |
| 6 | 66 |
| 7 | 72 |
| 8 | 78 |
| 9 | 82 |
| 10 | 87 |
| 11 | 90 |
| 12 | 93 |
| 13 | 95 |
| 14 | 97 |
| 15 | 99 |
| 16 | 100 |
| 17 | 100 |
| 18 | 101 |
| 19 | 101 |
| 20 | 102 |

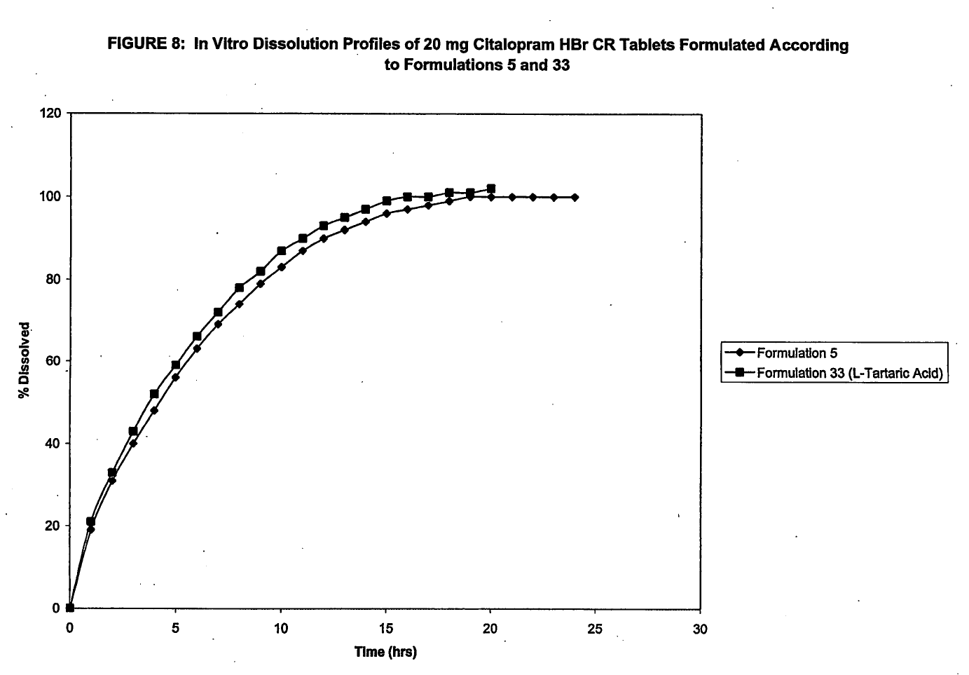

FIG. 8 compares the in vitro dissolution profiles of 20 mg Citalopram HBr Tablets formulated according to Formulations 5 and 33.

### Example 6

Table 16 provides the composition of two Citalopram HBr CR Tablet formulations comprising different concentrations of polyvinyl pyrrolidone (KOLLIDON® 90F) formulated to test an increase in the amount of polyvinyl pyrrolidone concentration and to determine its effect on solubility.

TABLE 16
Citalopram HBr CR Tablet Formulations to Test the Increase in
the Amount of PVP

Formulation No.

| | 34 | 35 |
|---|---|---|
| Ingredient | (% w/w) | (% w/w) |
| Citalopram HBr | 6.25 | 6.25 |
| HPMC (METHOCEL ® K4MPrem | 30.00 | 30.00 |
| CR) | | |
| Lactose Anhydrous (DT) | 53.00 | 53.00 |
| MCC (AVICEL ® PH 101) | 7.25 | 10.25 |
| PVP (KOLLIDON ® 90F) | 2.5 | 3.5 |
| Magnesium Stearate | 1.00 | 1.00 |

In vitro dissolution studies were conducted on 20 mg Citalopram HBr CR Tablets formulated according to Formulations 34 and 35. Table 17 provides the in vitro dissolution data of 20 mg Citalopram HBr CR Tablets formulated according to Formulations 34 and 35.

TABLE 17
In vitro Dissolution Data of 20 mg Citalopram HBr CR Tablets
Formulated According to Formulations 34 and 35

| | Formulation 34 | Formulation 35 |
|---|---|---|
| | (2.5% PVP) | (3.5% PVP) |
| Time (hrs) | (% dissolved) | (% dissolved) |
| 0 | 0 | 0 |
| 1 | 21 | 21 |
| 2 | 33 | 33 |
| 3 | 43 | 42 |
| 4 | 52 | 51 |
| 5 | 60 | 59 |
| 6 | 66 | 65 |
| 7 | 72 | 71 |
| 8 | 77 | 76 |
| 9 | 82 | 80 |
| 10 | 85 | 84 |
| 11 | 89 | 87 |
| 12 | 91 | 90 |
| 13 | 94 | 92 |
| 14 | 96 | 94 |
| 15 | 97 | 96 |
| 16 | 98 | 97 |
| 17 | 99 | 98 |
| 18 | 100 | 99 |
| 19 | 100 | 99 |
| 20 | 101 | 99 |
| 21 | | 100 |

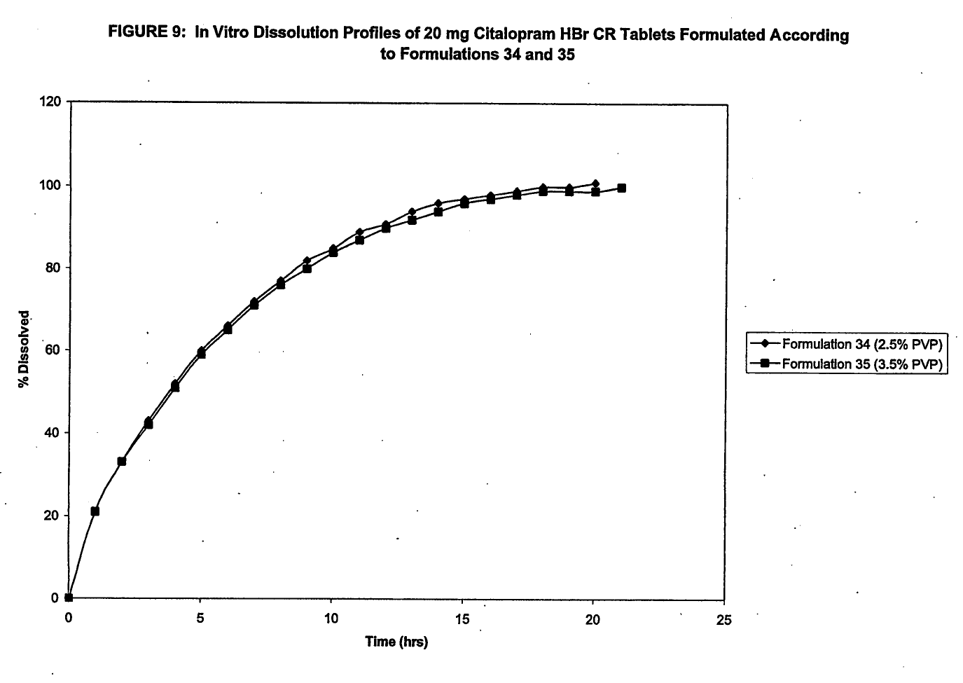

FIG. 9 compares the in vitro dissolution profiles of 20 mg Citalopram HBr CR Tablets formulated according to Formulations 34 and 35.

### Example 7

Table 18 provides the composition of two Citalopram HBr CR Tablet formulations; one formulated with Poloxamer 407 (PLURONIC® F127) (Formulation 36) and one formulated with PEG8000 (Formulation 37).

TABLE 18
Citalopram HBr CR Tablet Formulations to Compare the Effect of
Poloxamer 407 vs. PEG-8000

| | Formulation 36 | Formulation 37 |
|---|---|---|
| Core Ingredients | (g) | (g) |
| Citalopram HBr | 187.5 | 187.5 |
| HPMC (METHOCEL ® | 900.0 | 780.0 |
| PremK4M CR) | | |
| Lactose Anhydrous (DT) | 1440.0 | 1560.0 |
| MCC (AVICEL ® PH 101) | 217.5 | 217.5 |
| Poloxamer 407 | 45.0 | NA |
| (PLURONIC ® F127) | | |
| PEG 8000 | NA | 150.0 |
| PVP (KOLLIDON ® 90F) | 75.0 | 75.0 |
| Magnesium stearate | 30.0 | 30.0 |
| Water* | — | — |
| ISP* | — | — |
| Coating Ingredients | | |
| OPADRY ® II White Y-22- | 60 | 60 |
| 7719 | | |

*Alcohol and Water are not considered as part of the theoretical batch size since they are evaporated during drying of the wet granulation.

In vitro dissolution studies were conducted on 20 mg Citalopram HBr CR Tablets formulated according to Formulations 36 and 37. Table 19 provides the in vitro dissolution data of 20 mg Citalopram HBr CR Tablets formulated according to Formulations 36 and 37.

TABLE 19
In vitro Dissolution Data of 20 mg Citalopram HBr CR Tablets
Formulated According to Formulations 36 and 37

| | Formulation 36 | Formulation 37 |
|---|---|---|
| Time (Hrs) | (Poloxamer 407) | (PEG-8000) |
| 0 | 0 | 0 |
| 1 | 18 | 20 |
| 2 | 28 | 31 |
| 3 | 36 | 41 |
| 4 | 43 | 50 |
| 5 | 49 | 57 |
| 6 | 55 | 64 |
| 7 | 61 | 71 |
| 8 | 66 | 76 |
| 9 | 71 | 81 |
| 10 | 75 | 85 |
| 11 | 79 | 88 |
| 12 | 82 | 91 |
| 13 | 86 | 93 |
| 14 | 88 | 95 |
| 15 | 91 | 97 |
| 16 | 92 | 98 |
| 17 | 94 | 99 |
| 18 | 95 | 100 |
| 19 | 97 | 100 |
| 20 | 98 | 100 |
| 21 | 98 | |
| 22 | 99 | |
| 23 | 99 | |
| 24 | 100 | |

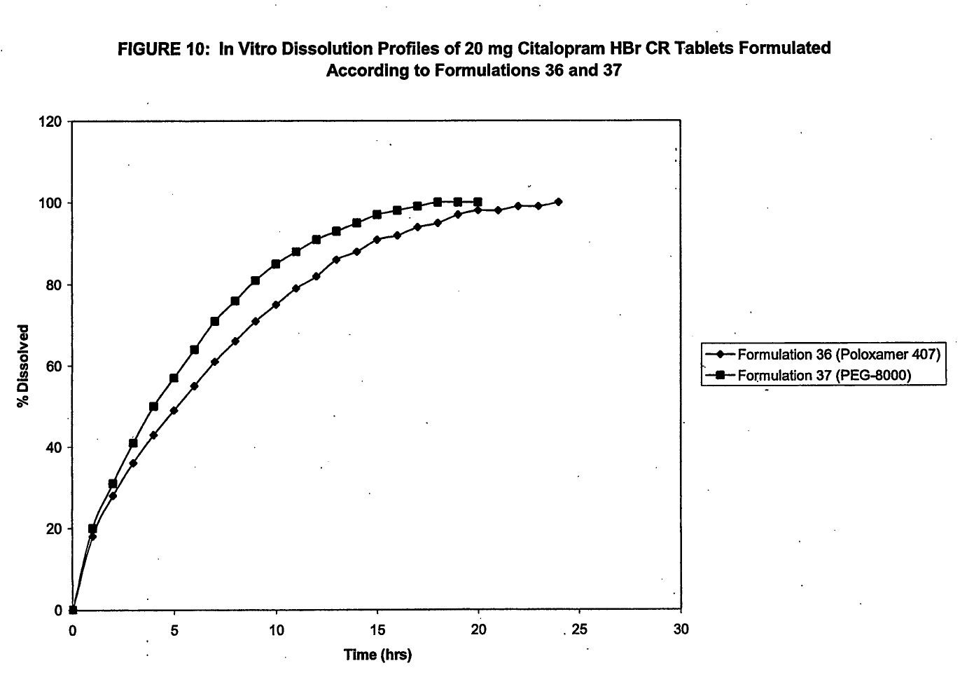

FIG. 10 compares the in vitro dissolution profiles of 20 mg Citalopram HBr CR Tablets formulated according to Formulations 36 and 37.

A randomized, three-way, single-dose, fasting, cross-over study was conducted to evaluate the bioavailability of 20 mg Citalopram HBr CR Tablets formulated according to Formulations 36 and 37 versus the reference product, 20 mg CELEXA™ tablets (Lundbeck Canada Inc.—Lot #: A304; Expiry Date: 04/MR) under fasting conditions in normal, healthy, non-smoking male volunteers. There was a one-(1) week washout period between the three study periods. Following each drug administration, blood samples were taken at 0.0 (pre-drug), 1.0, 9.0, 3.0, 3.5, 4.0, 4.5, 5.0, 5.5, 6.0, 8.0, 10.0, 12.0, 16.0, 24.0, 36.0, 48.0, 72.0, 96.0, 120.0 and 144.0 hours post-drug administration. Upon completion of the clinical portion of the study, all plasma samples were transferred to the analytical facility for analysis. Fifteen (15) qualified subjects and three (3) alternates were entered into the study. Laboratory analysis, and pharmacokinetic and statistical analyses were conducted on the first fifteen (15) evaluable subjects in a balanced group who completed the study.

Table 20 provides the mean plasma Citalopram concentrations (ng/ml) over time after a single dose of one 20 mg Citalopram HBr Tablet formulated according to Formulation 36 or 37 or after a single dose of one 20 mg CELEXA™ tablet under fasting conditions.

TABLE 20
Mean Plasma Citalopram Concentrations (n = 15)

| | Formulation 36 | Formulation 37 | CELEXA ™ |
|---|---|---|---|
| Sample Time | 1 × 20 mg | 1 × 20 mg | 1 × 20 mg |
| (Hours) | (ng/ml) | (ng/ml) | (ng/ml) |
| 0.00 | 0.74 ± 0.69 | 0.77 ± 0.92 | 0.51 ± 0.41 |
| 1.00 | 0.87 ± 0.76 | 0.91 ± 0.96 | 3.16 ± 2.36 |
| 2.00 | 2.18 ± 0.97 | 2.30 ± 1.10 | 11.77 ± 3.69 |
| 3.00 | 4.66 ± 1.78 | 4.78 ± 1.95 | 16.94 ± 3.43 |
| 3.50 | 5.85 ± 1.69 | 6.38 ± 2.40 | 16.89 ± 2.98 |
| 4.00 | 7.67 ± 2.80 | 7.78 ± 3.03 | 17.81 ± 3.30 |
| 4.50 | 8.75 ± 2.65 | 8.99 ± 3.20 | 18.29 ± 2.81 |
| 5.00 | 10.93 ± 2.99 | 11.44 ± 4.65 | 18.82 ± 3.30 |
| 5.50 | 12.45 ± 4.23 | 13.76 ± 5.18 | 18.87 ± 2.78 |
| 6.00 | 13.45 ± 3.94 | 14.56 ± 4.47 | 19.22 ± 2.70 |
| 8.00 | 14.72 ± 3.36 | 15.47 ± 3.42 | 17.88 ± 2.76 |
| 10.00 | 14.20 ± 3.07 | 15.30 ± 3.25 | 16.59 ± 2.88 |
| 12.00 | 14.41 ± 3.26 | 15.32 ± 3.56 | 15.69 ± 2.62 |
| 16.00 | 12.79 ± 2.81 | 13.60 ± 3.20 | 13.51 ± 1.66 |
| 24.00 | 10.14 ± 2.80 | 10.62 ± 2.97 | 11.04 ± 1.78 |
| 36.00 | 8.76 ± 2.95 | 9.29 ± 3.02 | 9.14 ± 1.77 |
| 48.00 | 6.69 ± 2.38 | 7.26 ± 2.61 | 7.15 ± 1.67 |
| 72.00 | 4.19 ± 1.74 | 4.35 ± 1.55 | 4.49 ± 1.19 |
| 96.00 | 2.82 ± 1.42 | 2.98 ± 1.33 | 3.05 ± 0.98 |
| 120.00 | 1.99 ± 1.13 | 1.92 ± 0.99 | 2.11 ± 0.81 |
| 144.00 | 1.43 ± 0.94 | 1.47 ± 0.94 | 1.52 ± 0.67 |

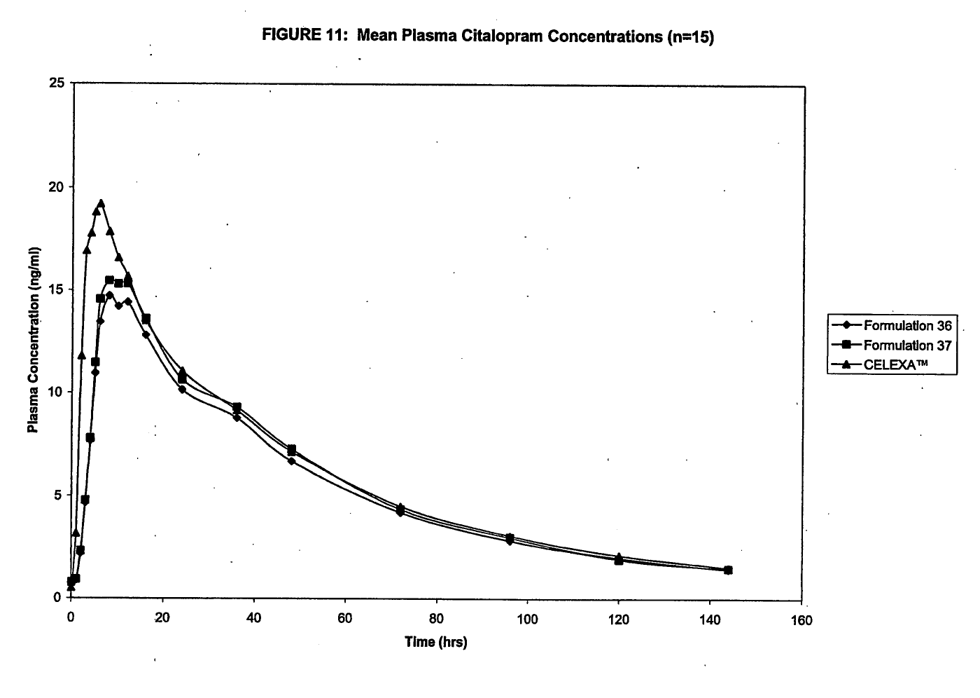

FIG. 11 illustrates the mean plasma Citalopram concentrations (ng/ml) over time after a single dose of one 20 mg Citalopram HBr CR Tablet formulated according to Formulation 36 or 37 or after a single dose of one product 20 mg CELEXA™ tablet under fasting conditions.

Table 21 provides the mean pharmacokinetic parameters for plasma Citalopram after a single dose of one 20 mg Citalopram HBr CR Tablet formulated according to Formulation 36 or 37 or after a single dose of one 20 mg CELEXA™ tablet under fasting conditions.

TABLE 21
Mean Pharmacokinetic Parameters for Plasma Citalopram (n = 15)

| | Geometric Mean | | |
|---|---|---|---|
| | Arithmetic Mean (% C.V.) | | |
| | Formulation 36 | Formulation 37 | CELEXA ™ |
| Parameter | 1 × 20 mg | 1 × 20 mg | 1 × 20 mg |
| AUC_{(O - t hours)} | 748.98 | 799.78 | 883.21 |
| (ng · hr/ml) | 785.59 (32.94) | 830.67 (31.78) | 896.80 (18.48) |
| AUC_{(O - infinity)} | 832.37 | 890.39 | 984.00 |
| (ng · hr/ml) | 886.63 (39.08) | 942.57 (40.05) | 1007.02 (22.89) |
| Cₘₐₓ (ng/ml) | 15.12 | 16.22 | 19.93 |
| | 15.42 (20.08) | 16.62 (24.62) | 20.13 (14.28) |
| Tₘₐₓ (hours)** | 10.00 (2.26) | 8.00 (2.26) | 5.50 (1.98) |
| t_{1/2} (hours)* | 43.71 (20.63) | 45.68 (28.77) | 46.11 (28.60) |
| Kₑₗ (hour⁻¹)* | 0.016 (18.240) | 0.016 (21.295) | 0.016 (21.932) |

*These are arithmetic means (%.C.V.)
**This is median (±SD)

### Example 8

Table 22 provides the composition of a Citalopram HBr CR Tablet formulation (Formulation 38) based on the ingredients in Formulation 5 with adjustments to the amounts of the rate controlling polymer, HPMC and Lactose Anhydrous (DT).

TABLE 22
Citalopram HBr CR Tablet Formulation

| | Formulation 38 |
|---|---|
| Ingredients | (% w/w) |
| Citalopram HBr | 6.31 |
| HPMC (METHOCEL ® K4MPrem CR) | 27.31 |
| Lactose Anhydrous (DT) | 56.56 |
| MCC (AVICEL ® PH 101) | 8.08 |
| PVP (KOLLIDON ® 29/32) | NA |
| PVP (KOLLIDON ® K90F) | 1.77 |
| Magnesium Stearate | 0.99 |
| Isopropyl Alcohol* | — |

*Evaporated

### Example 9

Table 23 provides the composition of a Citalopram HBr CR Tablet formulation (Formulation 39) based on the ingredients in Formulation 38 with only minor adjustment in the amounts of HPMC and Lactose Anhydrous (DT).

TABLE 23
Citalopram HBr CR Tablet Formulation

| | Formulation 39 |
|---|---|
| Ingredients | (% w/w) |
| Citalopram HBr | 6.31 |
| HPMC (METHOCEL ® K4MPrem CR) | 26.51 |
| Lactose Anhydrous (DT) | 57.31 |
| MCC (AVICEL ® PH 101) | 8.00 |
| PVP (KOLLIDON ® K90F) | 1.77 |
| Magnesium Stearate | 0.99 |
| Isopropyl Alcohol | * |

In vitro dissolution studies were conducted on 20 mg Citalopram HBr CR Tablets formulated according to Formulation 39. Table 24 provides the in vitro dissolution data of 20 mg Citalopram HBr CR Tablets formulated according to Formulation 39.

TABLE 24
Citalopram HBr CR Tablet Formulation

| | Formulation 39 |
|---|---|
| Time (Hrs) | (% dissolved) |
| 0 | 0 |
| 1 | 23.17 |
| 2 | 35.81 |
| 3 | 46.01 |
| 4 | 54.94 |
| 5 | 62.74 |
| 6 | 69.62 |
| 7 | 75.53 |
| 8 | 80.66 |
| 9 | 85.04 |
| 10 | 88.75 |
| 11 | 92.03 |
| 12 | 94.76 |
| 13 | 97.07 |
| 14 | 98.82 |
| 15 | 100.22 |
| 16 | 101.46 |
| 17 | 102.55 |
| 18 | 103.44 |
| 19 | 104.36 |
| 20 | 105.23 |

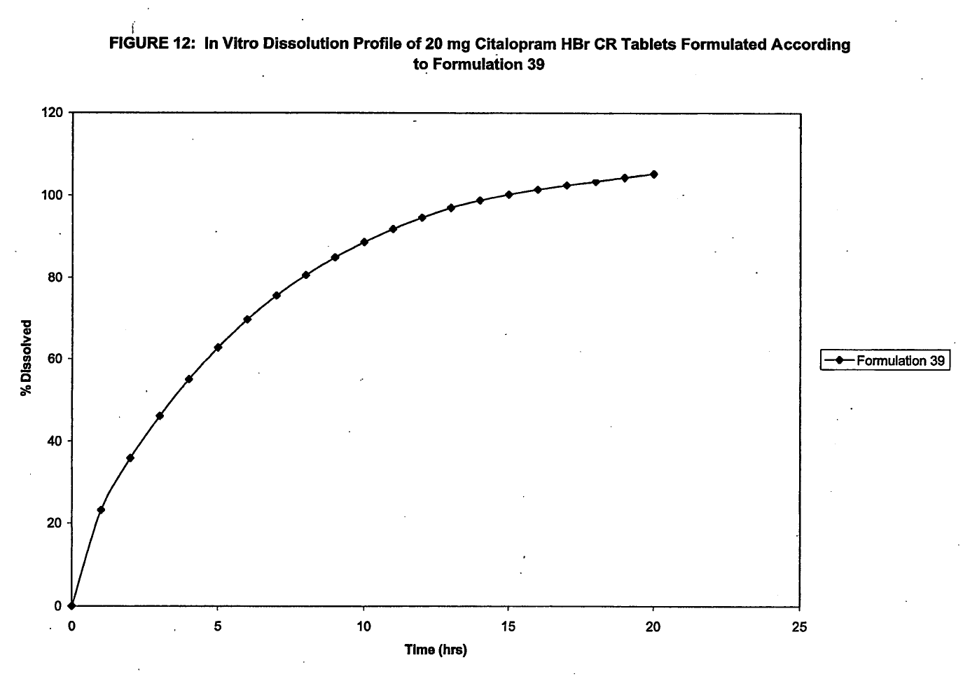

FIG. 12 illustrates the in vitro dissolution profile of 20 mg Citalopram HBr CR Tablets formulated according to Formulation 39.

A randomized, two-way, single-dose, blinded, cross-over design study was conducted to evaluate the bioavailability of 20 mg Citalopram HBr CR Tablets formulated according, to Formulation 39 relative to 20 mg CELEXA™ tablets under fasting conditions in normal, healthy, non-smoking male volunteers. The study periods were separated by a four—(4) week washout period. Blood sampling for drug content analysis was carried out at 0.0 (pre-drug), 1.0, 1.5, 2.0, 2.5, 3.0, 4.0, 5.0, 6.0, 7.0, 8.0, 10.0, 12.0, 14.0, 16.0, 20.0, 24.0, 36.0, 48.0, 72.0, 96.0, 120.0, 144.0, 168.0, 192.0, 216.0, 240.0 hours post-drug administration. Upon completion of the clinical portion of the study, all plasma samples were delivered to the analytical facility for the determination of the concentrations of Citalopram and its metabolites, Desmethylcitalopram (DCT) and Didesmethylcitalopram (DDCT) in the samples. Sixty-two (62) qualified subjects were entered into the study. Pharmacokinetic and statistical analyses were conducted on fifty (50) evaluable subjects that completed the study.

Table 25 provides the mean plasma Citalopram concentrations (ng/ml) over time after a single dose of one 20 mg Citalopram HBr CR Tablet formulated according to Formulation 39 or after a single dose of one 20 mg CELEXA™ tablet under fasting conditions.

TABLE 25
Mean Plasma Citalopram Concentrations (n = 50)

| | Formulation 39 | CELEXA ™ |
|---|---|---|
| | 1 × 20 mg | 1 × 20 mg |
| Time (hrs) | (ng/ml) | (ng/ml) |
| 0 | 0.00 ± 0.00 | 0.00 ± 0.00 |
| 1 | 0.21 ± 0.24 | 2.59 ± 2.13 |
| 1.5 | 0.91 ± 0.56 | 7.98 ± 5.00 |
| 2 | 2.04 ± 1.10 | 12.55 ± 5.37 |
| 2.5 | 3.46 ± 1.47 | 15.83 ± 5.32 |
| 3 | 5.00 ± 1.79 | 17.62 ± 5.08 |
| 4 | 8.82 ± 3.35 | 19.50 ± 4.54 |
| 5 | 12.98 ± 3.96 | 19.98 ± 4.01 |
| 6 | 16.17 ± 4.86 | 20.15 ± 4.06 |
| 7 | 17.57 ± 4.83 | 19.63 ± 3.92 |
| 8 | 17.60 ± 4.73 | 18.96 ± 3.74 |
| 10 | 17.07 ± 4.41 | 17.62 ± 3.66 |
| 12 | 16.35 ± 4.13 | 16.09 ± 3.43 |
| 14 | 15.73 ± 4.02 | 15.76 ± 3.28 |
| 16 | 14.27 ± 3.86 | 14.62 ± 3.39 |
| 20 | 12.94 ± 3.34 | 13.29 ± 3.01 |
| 24 | 11.73 ± 3.11 | 11.74 ± 2.81 |
| 36 | 9.98 ± 3.07 | 9.95 ± 2.64 |
| 48 | 8.09 ± 2.59 | 8.05 ± 2.29 |
| 72 | 5.11 ± 1.97 | 5.08 ± 2.08 |
| 96 | 3.53 ± 1.66 | 3.51 ± 1.65 |
| 120 | 2.53 ± 1.34 | 2.46 ± 1.46 |
| 144 | 1.78 ± 1.01 | 1.80 ± 1.23 |
| 168 | 1.31 ± 0.85 | 1.27 ± 0.86 |
| 192 | 0.95 ± 0.67 | 0.94 ± 0.70 |
| 216 | 0.71 ± 0.58 | 0.67 ± 0.57 |
| 240 | 0.50 ± 0.46 | 0.51 ± 0.48 |

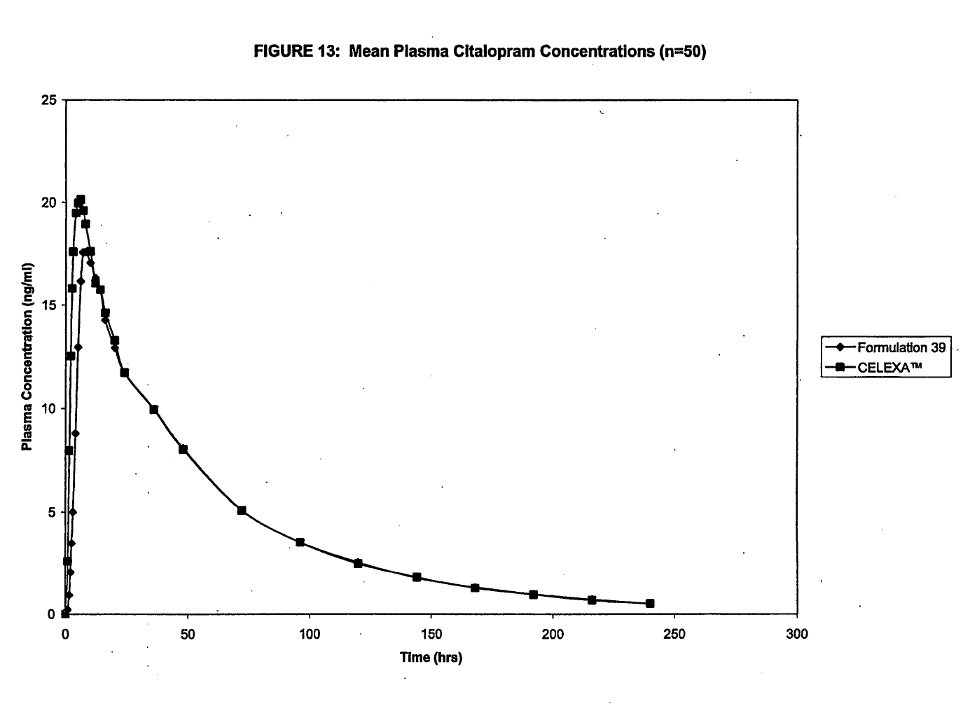

FIG. 13 illustrates the mean plasma Citalopram concentrations (ng/ml) over time after a single dose of one 20 mg Citalopram HBr CR Tablet formulated according to Formulation 39 or after a single dose of one 20 mg CELEXA™ tablet under fasting conditions.

Table 26 provides the mean pharmacokinetic parameters for plasma Citalopram after a single dose of one 20 mg Citalopram HBr CR Tablet formulated according to Formulation 39 or after a single dose of one 20 mg CELEXA™ tablet.

TABLE 26
Mean Pharmacokinetic Parameters for Plasma Citalopram (n = 50)

| | Geometric Mean | | Ratio of |
|---|---|---|---|
| | Arithmetic Mean (±SD) | | Geometric |
| | Formulation 39 | CELEXA ™ | Means (%) |
| Parameter | 1 × 20 mg | 1 × 20 mg | Test/Reference |
| AUC_{(O-t hours)} | 953.60 | 1015.36 | 94% |
| (ng · hr/ml) | 1016.14 ± 353.93 | 1066.22 ± 341.50 | |
| AUC_{(0-24 hours)} | 295.89 | 354.25 | 84% |
| (ng · hr/ml) | 305.41 ± 76.53 | 360.43 ± 73.33 | |
| AUC_{(O-infinity)} | 1002.55 | 1056.69 | 94% |
| (ng · hr/ml) | 1073.17 ± 388.23 | 1115.12 ± 381.10 | |
| Cₘₐₓ (ng/ml) | 17.96 | 21.22 | 85% |
| | 18.56 ± 4.64 | 21.64 ± 4.19 | |
| Tₘₐₓ (hours)** | 8.00 ± 1.81 | 6.00 ± 2.46 | N/A |
| t½ (hours)* | 52.63 ± 11.25 | 53.23 ± 11.21 | N/A |
| Kₑₗ (hour⁻¹)* | 0.014 ± 0.003 | 0.014 ± 0.003 | N/A |

*These are arithmetic means (±SD)
**This is median (±SD)

Table 27 provides the comparison of plasma Citalopram data after a single dose of one 20 mg Citalopram HBr CR Tablet formulated according to Formulation 39 versus after a single dose of one 20 mg CELEXA™ tablet under fasting conditions (Uncorrected).

TABLE 27
Comparison of Plasma Citalopram Data (Formulation 39 vs.
CELEXA ™ - Uncorrected)

| | AUC_{(0-t hours)} | AUC_{(O-infinity)} | Cₘₐₓ |
|---|---|---|---|
| 90% Geometric C.I.¹ | 90%-97% | 91%-98% | 81%-88% |
| Ratio of Means² | 94% | 94% | 85% |
| 95% Geometric C.I.³ | 90%-98% | 90%-98% | 80%-89% |
| Westlake's C.L.⁴ | 7% | 7% | 17% |

¹90% Geometric Confidence interval using log-transformed data and CELEXA ™ (Lundbeck Canada Inc.) as the reference.
²Calculated using geometric means according to the formula: e^{(Formulation 39) * (CELEXA ™)} × 100%.
³95% Geometric Confidence interval using log-transformed data and CELEXA ™ (Lundbeck Canada Inc.) as the reference.
⁴Confidence Limit using Westlake's method and CELEXA ™ (Lundbeck Canada Inc.) as the reference.

Table 28 provides the comparison of plasma Citalopram data after a single dose of one 20 mg Citalopram HBr CR Tablet formulated according to Formulation 39 versus after a single dose of one 20 mg CELEXA™ tablet under fasting conditions (Potency Corrected).

TABLE 28
Comparison of Plasma Citalopram Data (Formulation 39 vs.
CELEXA ™ - Potency Corrected)

| | |
|---|---|
| AUC_{(0-t hours)} | Cₘₐₓ |

| | | |
|---|---|---|
| 90% Geometric C.I.¹ | 86%-92% | 77%-84% |
| Ratio of Means² | 89% | 80% |

¹90% Geometric Confidence interval using log-transformed data and CELEXA ™ (Lundbeck Canada Inc.) as the reference.
²Calculated using geometric means according to the formula: e^{(Formulation 39) * (CELEXA ™)} × 100%.

Table 29 provides the mean plasma Desmethylcitalopram concentrations (ng/ml) over time after a single dose of one 20 mg Citalopram HBr CR Tablet formulated according to Formulation 39 or after a single dose of one 20 mg CELEXA™ tablet under fasting conditions.

TABLE 29
Mean Plasma Desmethylcitalopram Concentrations (n = 50)

| | Formulation 39 | CELEXA ™ |
|---|---|---|
| | 1 × 20 mg | 1 × 20 mg |
| Time (hrs) | (ng/ml) | (ng/ml) |
| 0 | 0 | 0 |
| 1 | 0.02 | 0.34 |
| 1.5 | 0.09 | 0.8 |
| 2 | 0.2 | 1.19 |
| 2.5 | 0.32 | 1.39 |
| 3 | 0.45 | 1.51 |
| 4 | 0.76 | 1.75 |
| 5 | 1.13 | 1.92 |
| 6 | 1.36 | 2.07 |
| 7 | 1.62 | 2.22 |
| 8 | 1.74 | 2.32 |
| 10 | 2.01 | 2.52 |
| 12 | 2.1 | 2.47 |
| 14 | 2.17 | 2.55 |
| 16 | 2.15 | 2.5 |
| 20 | 2.16 | 2.56 |
| 24 | 2.08 | 2.37 |
| 36 | 2.37 | 2.57 |
| 48 | 2.34 | 2.48 |
| 72 | 1.98 | 1.98 |
| 96 | 1.61 | 1.64 |
| 120 | 1.28 | 1.27 |
| 144 | 1.01 | 0.99 |
| 168 | 0.77 | 0.76 |
| 192 | 0.61 | 0.61 |
| 216 | 0.49 | 0.48 |
| 240 | 0.39 | 0.38 |

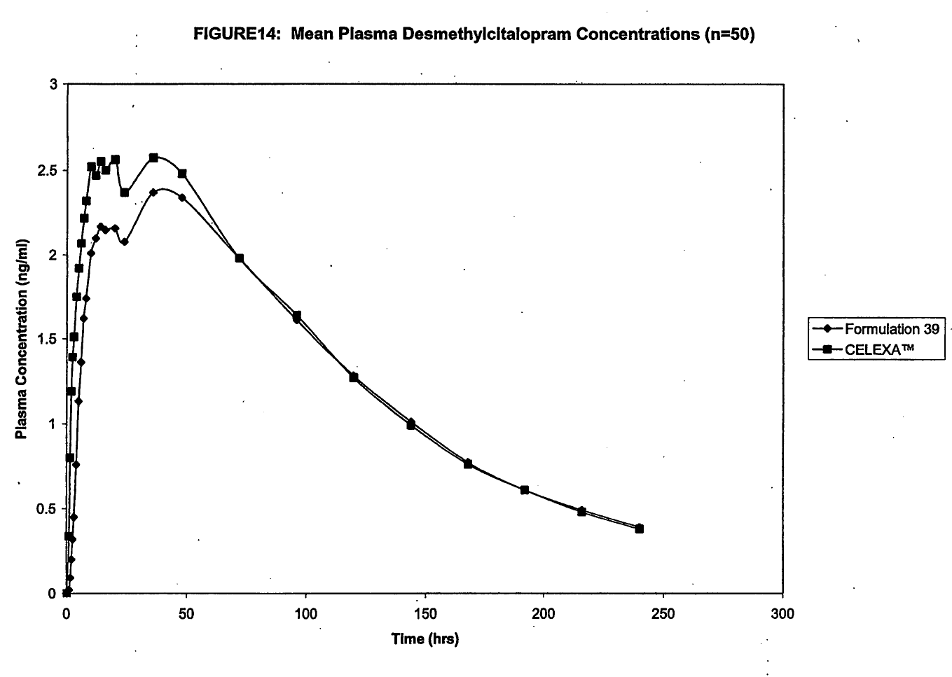

FIG. 14 illustrates the mean plasma Desmethylcitalopram concentrations (ng/ml) over time after a single dose of one 20 mg Citalopram HBr CR Tablet formulated according to Formulation 39 or after a single dose of one 20 mg CELEXA™ tablet under fasting conditions.

Table 30 provides the mean pharmacokinetic parameters for plasma Desmethylcitalopram after a single dose of one 20 mg Citalopram HBr CR Tablet formulated according to Formulation 39 or after a single dose of one 20 mg CELEXA™ tablet under fasting conditions.

TABLE 30
Mean Pharmacokinetic Parameters for Plasma
Desmethylcitalopram (n = 50)

| | Geometric Mean | | Ratio of |
|---|---|---|---|
| | Arithmetic Mean (±SD) | | Geometric |
| | Formulation 39 | CELEXA ™ | Means (%) |
| Parameter | 1 × 20 mg | 1 × 20 mg | Test/Reference |
| AUC_{(O-t hours)} (ng | 299.08 | 319.39 | 94% |
| hr/ml) | 310.24 ± 82.79 | 328.90 ± 78.49 | |
| AUC_{(0-24 hours)} | 298.87 | 320.54 | 77% |
| (ng · hr/ml) | 310.24 ± 82.79 | 328.90 ± 78.49 | |
| AUC_{(O-infinity)} | 339.75 | 359.42 | 94% |
| (ng · hr/ml | 356.61 ± 110.33 | 373.26 ± 104.14 | |
| Cₘₐₓ (ng/ml) | 2.46 | 2.76 | 89% |
| | 2.55 ± 0.65 | 2.86 ± 0.75 | |
| Tₘₐₓ (hours)** | 36.00 ± 14.72 | 20.00 ± 19.03 | N/A |
| t_{1/2} (hours)* | 70.20 ± 24.99 | 69.01 ± 28.32 | N/A |
| Kₑₗ (hour⁻¹)* | 0.011 ± 0.003 | 0.011 ± 0.003 | N/A |

*These are arithmetic means (±SD)
**This is median (±SD)

Table 31 provides the comparison of plasma Desmethylcitalopram data after, a single dose of one 20 mg Citalopram HBr CR Tablet formulated according to Formulation 39 versus after a single dose of one. 20 mg CELEXA™ tablet under fasting conditions (Uncorrected).

TABLE 31
Comparison of Plasma Desmethylcitalopram Data (Formulation
39 vs. CELEXA ™ - Uncorrected)

| | AUC_{(0-t hours)} | AUC_{(O-infinity)} | Cₘₐₓ |
|---|---|---|---|
| 90% Geometric C.I.¹ | 90%-97% | 91%-98% | 85%-93% |
| Ratio of Means² | 94% | 94% | 89% |
| 95% Geometric C.I.³ | 90%-98% | 90%-99% | 85%-93% |
| Westlake's C.L.⁴ | 8% | 7% | 14% |

¹90% Geometric Confidence interval using log-transformed data and CELEXA ™ (Lundbeck Canada Inc.) as the reference.
²Calculated using geometric means according to the formula: e^{(Formulation 39) * (CELEXA ™)} × 100%.
³95% Geometric Confidence interval using log-transformed data and CELEXA ™ (Lundbeck Canada Inc.) as the reference.
⁴Confidence Limit using Westlake's method and CELEXA ™ (Lundbeck Canada Inc.) as the reference.

Table 32 provides the comparison of plasma Desmethylcitalopram data after a single dose of one 20 mg Citalopram HBr CR Tablet formulated according to Formulation 39 versus after a single dose of one 20 mg CELEXA™ tablet under fasting conditions (Potency Corrected).

TABLE 32
Comparison of Plasma Desmethylcitalopram Data (Formulation
39 vs. CELEXA ™ - Potency Corrected)

| | AUC_{(0-t hours)} | Cₘₐₓ |
|---|---|---|
| 90% Geometric C.I.¹ | 86%-92% | 81%-88% |
| Ratio of Means² | 89% | 84% |

¹90% Geometric Confidence interval using log-transformed data and CELEXA ™ (Lundbeck Canada Inc.) as the reference.
²Calculated using geometric means according to the formula: e^{(Formulation 39) * (CELEXA ™)} × 100%.

Table 33 provides the mean plasma Didesmethylcitalopram concentrations (ng/ml) over time after a single dose of one 20 mg Citalopram HBr CR Tablet formulated according to Formulation 39 or after a single dose of one 20 mg CELEXA™ tablet under fasting conditions.

TABLE 33
Mean Plasma Didesmethylcitalopram Concentrations (n = 50)

| | Formulation 39 | CELEXA ™ |
|---|---|---|
| | 1 × 20 mg | 1 × 20 mg |
| Time (hrs) | (ng/ml) | (ng/ml) |
| 0 | 0 | 0 |
| 1 | 0 | 0.01 |
| 1.5 | 0 | 0.03 |
| 2 | 0 | 0.05 |
| 2.5 | 0 | 0.07 |
| 3 | 0.04 | 0.09 |
| 4 | 0.03 | 0.11 |
| 5 | 0.06 | 0.12 |
| 6 | 0.09 | 0.14 |
| 7 | 0.11 | 0.16 |
| 8 | 0.13 | 0.19 |
| 10 | 0.18 | 0.24 |
| 12 | 0.21 | 0.24 |
| 14 | 0.23 | 0.32 |
| 16 | 0.25 | 0.31 |
| 20 | 0.27 | 0.35 |
| 24 | 0.29 | 0.35 |
| 36 | 0.39 | 0.46 |
| 48 | 0.46 | 0.51 |
| 72 | 0.49 | 0.5 |
| 96 | 0.48 | 0.48 |
| 120 | 0.43 | 0.44 |
| 144 | 0.38 | 0.37 |
| 168 | 0.31 | 0.3 |
| 192 | 0.27 | 0.25 |
| 216 | 0.22 | 0.21 |
| 240 | 0.18 | 0.17 |

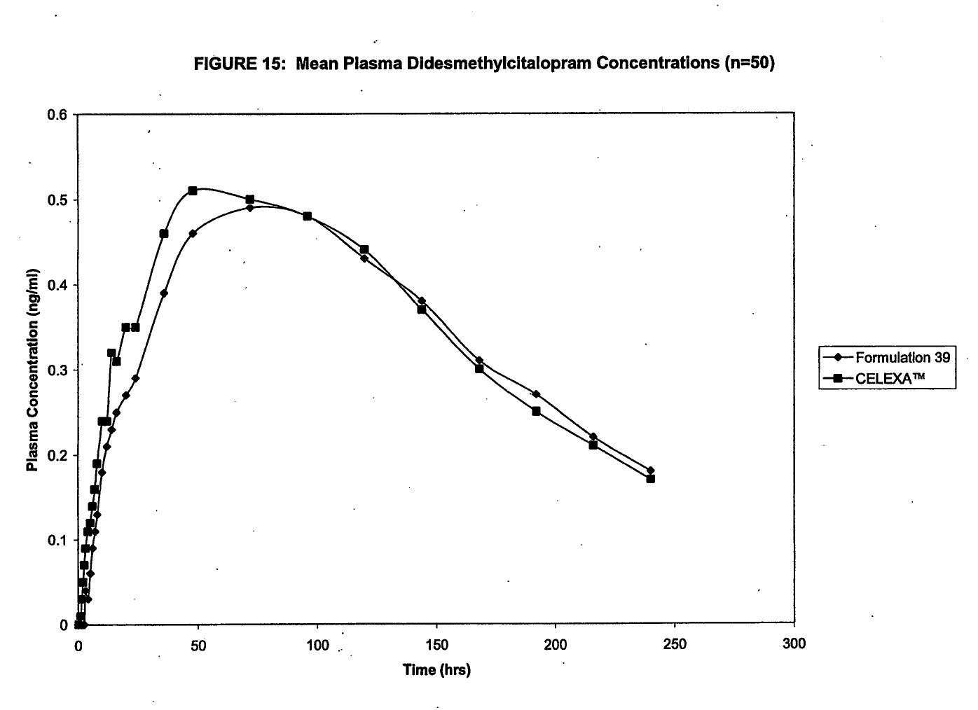

FIG. 15 illustrates the mean plasma Didesmethylcitalopram concentrations (ng/ml) over time after a single dose of one 20 mg Citalopram HBr CR Tablet formulated according to Formulation 39 or after a single dose of one 20 mg CELEXA™ tablet under fasting conditions.

Table 34 provides the mean pharmacokinetic parameters for plasma Didesmethylcitalopram after a single dose of one 20 mg Citalopram HBr CR Tablet formulated according to Formulation 39 or after a single dose of one 20 mg CELEXA™ tablet under fasting conditions.

TABLE 34
Mean Pharmacokinetic Parameters for Plasma Didesmethylcitalopram
(n = 50)

| | Geometric Mean | | Ratio of |
|---|---|---|---|
| | Arithmetic Mean (±SD) | | Geometric |
| | Formulation 39 | CELEXA ™ | Means (%) |
| Parameter | 1 × 20 mg | 1 × 20 mg | Test/Reference |
| AUC_{(O-t hours)} | 74.43 | 64.67 | 99% |
| (ng hr/ml) | 82.96 ± 45.63 | 85.22 ± 49.10 | |
| AUC_{(0-24 hours)} | 3.49 | 3.63 | 80% |
| (ng · hr/ml) | 4.17 ± 2.66 | 5.48 ± 3.83 | |
| AUC_{(O-infinity)} | 111.92 | 103.74 | 98% |
| (ng · hr/ml) | 356.61 ± 110.33 | 373.26 ± 104.14 | |
| Cₘₐₓ (ng/ml) | 0.50 | 0.49 | 99% |
| | 0.57 ± 0.32 | 0.60 ± 0.37 | |
| Tₘₐₓ (hours)** | 84.00 ± 37.03 | 72.00 ± 35.04 | N/A |
| t_{1/2} (hours)* | 84.90 ± 29.41 | 97.78 ± 60.73 | N/A |
| Kₑₗ (hour⁻¹) | 0.009 ± 0.003 | 0.009 ± 0.003 | N/A |

*These are arithmetic means (±SD)
**This is median (±SD)

Table 35 provides the comparison of plasma Didesmethylcitalopram data after a single dose of one 20 mg Citalopram HBr CR Tablet formulated according to Formulation 39 versus after a single dose of one 20 mg CELEXA™ tablet under fasting conditions (Uncorrected).

TABLE 35
Comparison of Plasma Didesmethylcitalopram Data
(Formulation 39 vs. CELEXA ™ - Uncorrected)

| | AUC_{(0-t hours)} | AUC_{(0-infinity)} | Cₘₐₓ |
|---|---|---|---|
| 90% Geometric C.I.¹ | 94%-103% | 89%-108% | 93%-105% |
| Ratio of Means² | 99% | 98% | 99% |
| 95% Geometric C.I.³ | 93% 104% | 87%-110% | 91%-107% |
| Westlake's C.L.⁴ | 7% | 10% | 10% |

¹90% Geometric Confidence interval using log-transformed data and CELEXA ™ (Lundbeck Canada Inc.) as the reference.
²Calculated using geometric means according to the formula: e^{(Formulation 39) * (CELEXA ™)} × 100%.
³95% Geometric Confidence interval using log-transformed data and CELEXA ™ (Lundbeck Canada Inc.) as the reference.
⁴Confidence Limit using Westlake's method and CELEXA ™ (Lundbeck Canada Inc.) as the reference.

Table 36 provides the comparison of plasma Didesmethylcitalopram data after a single dose of one 20 mg Citalopram HBr CR Tablet formulated according to Formulation 39 versus after a single dose of one 20 mg CELEXA™ tablet under fasting conditions (Potency Corrected).

TABLE 36
Comparison of Plasma Didesmethylcitalopram Data
(Formulation 39 vs. CELEXA ™ - Potency Corrected)

| | AUC_{(0-t hours)} | Cₘₐₓ |
|---|---|---|
| 90% Geometric C.I.¹ | 89%-98% | 88%-100% |
| Ratio of Means² | 94% | 94% |

¹90% Geometric Confidence interval using log-transformed data and CELEXA ™ (Lundbeck Canada Inc.) as the reference.
²Calculated using geometric means according to the formula: e^{(Formulation 39) * (CELEXA ™)} × 100%.

Based on the 20 mg Citalopram HBr CR Tablet formulated according to Formulation 39 to the 20 mg CELEXA™ tablet comparison of Citalopram and its metabolites, Desmethylcitalopram (DCT) and Didesmethylcitalopram (DDCT) plasma data, the 90% geometric confidence intervals for AUC₍₀₋ₜ₎, AUC_{(0-infinity)} and Cₘₐₓ were found to be within the 80%-125% range and, therefore, the 20 mg Citalopram HBr CR Tablets formulated according to Formulation 39 are bioequivalent to the 20 mg CELEXA™ tablets. Also, based on the general linear model for T there is a significant difference between the 20 mg Citalopram HBr CR Tablets formulated according to Formulation 39 and the 20 mg CELEXA™ tablets (Lundbeck Canada Inc., Lot Number: A 304; Expiry Dam: 04/MR) under fasting conditions.

The potency correction for the actual drug strengths had the effect of shifting the uncorrected data to lower values, without affecting the conclusions for bioequivalence, in the measured data. That is, for the AUC₍₀₋ₜ₎, the 90% geometric confidence intervals (C.I.),were shifted from 900%-97% to 86%-92%, and the relative ratio of the means was shifted from 94% to 89%. Similarly, for the Cₘₐₓ parameter, the relative ratio of the means was reduced from 85% to 80%. Thus, the potency corrected data still meets the standards for bioequivalence for both the measured and potency corrected data.

### Example 10

Citalopram HBr was provided by two different suppliers (Supplier 1 and Supplier 2). Particle size measurements were conducted on Citalopram HBr provided by Supplier 1 and Supplier 2. Tables 37A and 37B provide the particle size measurement data of Citalopram HBr provided by Supplier 1 and Supplier 2, respectively.

TABLE 37A
Particle Size Measurement Data of Citalopram HBr Provided by
Supplier 1
Condition

| | | | | | |
|---|---|---|---|---|---|
| T % | 97.7% | Dist. Form: | Std. | Sampling times: | 1 [symbol] |
| (He—Ne): | | | | | |
| Trigger: | NO | R.R. Index: | 1.64-0.00i | | |
| Shaker: | 80 | Air P.: | HIGH | Vacuum: | [symbol] |

Format

| | | | | | |
|---|---|---|---|---|---|
| Dist. Base: | Volume | Scalling: | Auto | Axis: | LogX − Linear Y |

Data

| | | | | | |
|---|---|---|---|---|---|
| Median: | 7.781□m | SP. Area: | 10290 cm²/cm² | S.D.: | 4.704□m |
| Mode: | 8,253□m | Mean: | 8.569□m | | |
| C.V.: | 54.90% | | | | |

Span: (D 20.0-D 10.0)/D50 = 0.172

| | | | | | |
|---|---|---|---|---|---|
| Dia.on % | (90.0%): | 14.874□m | % on Dia. | (1.000□m): | 0. [symbol] |
| Dia.on % | (70.0%): | 10.295□m | % on Dia. | (5.000□m): | 23. [symbol] |
| Dia.on % | (35.0%): | 6.212□m | % on Dia. | (10.000□m): | 68. [symbol] |
| Dia.on % | (20.0%): | 4.606□m | % on Dia. | (15.000□m): | 90. [symbol] |
| Dia.on % | (10.0%): | 3.267□m | % on Dia. | (20.000□m): | 97. [symbol] |

[symbol]

indicates data missing or illegible when filed

TABLE 37B
Particle Size Measurement Data of Citalopram HBr Provided by
Supplier 2
System Details

Dispersed in air
1%-12% Obscuration, 1000 sweeps (100, 1 000 mm lenses)

| | | |
|---|---|---|
| Range Lens: 100 mm | Beam Length: 10.00 mm | Sampler: MS64 |
| Obscuration: 6.1% | | |
| Presentation: 2RHA | [Particle R.I = (1.4500, 0.1000); | Dispersant R.I. = 1.000)] |
| Analysis Model: Polydisperse | | Residual: |
| Modifications: Active - | Blended with Record 3; | |

Result Statistics

| | | |
|---|---|---|
| Distribution Type: Volume | Concentration = 0.0005% Vol | Uniformity = 3.518E+01 |

D (v. 0.1) = 1.62 □mMedian, D (v, 0.5) = 7.27 □m D (V, 0.9) = 1191.10 □m

| | |
|---|---|
| Mean, D [4, 3] = 259.27 □m | Span = 1.637E+02 |

Table 38 provides the composition of two Citalopram HBr CR Tablet formulations wherein the Citalopram HBr is provided by two different suppliers [Formulation 40 (Supplier 1) and Formulation 41 (Supplier 2)].

TABLE 38
Citalopram HBr CR Tablet Formulations

| | Formulation 40 | Formulation 41 (Supplier |
|---|---|---|
| | (Supplier 1) | 2) |
| Ingredients | (% w/w) | (% w/w) |
| Citalopram HBr | 6.25 | 6.25 |
| HPMC (K4MPrem CR) | 26.25 | 26.25 |
| Lactose Anhydrous | 56.75 | 56.75 |
| MCC | 8.00 | 8.00 |
| PVP (90F) | 1.75 | 1.75 |
| Magnesium Stearate | 1.00 | 1.00 |

In vitro dissolution studies were conducted on 20 mg Citalopram HBr CR Tablets formulated according Formulations 40 and 41. Table 39 provides the in vitro dissolution data of 20 mg Citalopram HBr CR Tablets formulated according to Formulations 40 and 41.

TABLE 39
In vitro Dissolution Data of 20 mg Citalopram HBr Tablets
Formulated According to Formulations 40 and 41

| | | Formulation 41 (Supplier |
|---|---|---|
| | Formulation 40 (Supplier 1) | 2) |
| Time (hrs) | (% dissolved) | (% dissolved) |
| 0 | 0 | 0 |
| 1 | 21.46 | 16.8 |
| 2 | 34.47 | 30.6 |
| 3 | 45.28 | 40.2 |
| 4 | 54.69 | 49.6 |
| 5 | 63.11 | 57.3 |
| 6 | 70.44 | 64.4 |
| 7 | 76.49 | 71 |
| 8 | 81.94 | 77.3 |
| 9 | 86.59 | 82.6 |
| 10 | 90.43 | 87.1 |
| 11 | 93.66 | 91 |
| 12 | 96.33 | 94.4 |
| 13 | 98.54 | 97.2 |
| 14 | 100.51 | 99.4 |
| 15 | 103.68 | 101.1 |
| 16 | 104.46 | 102.5 |
| 17 | 104.04 | 103.8 |
| 18 | 104.33 | 104.3 |
| 19 | 104.49 | 104.2 |
| 20 | 104.92 | 105.2 |

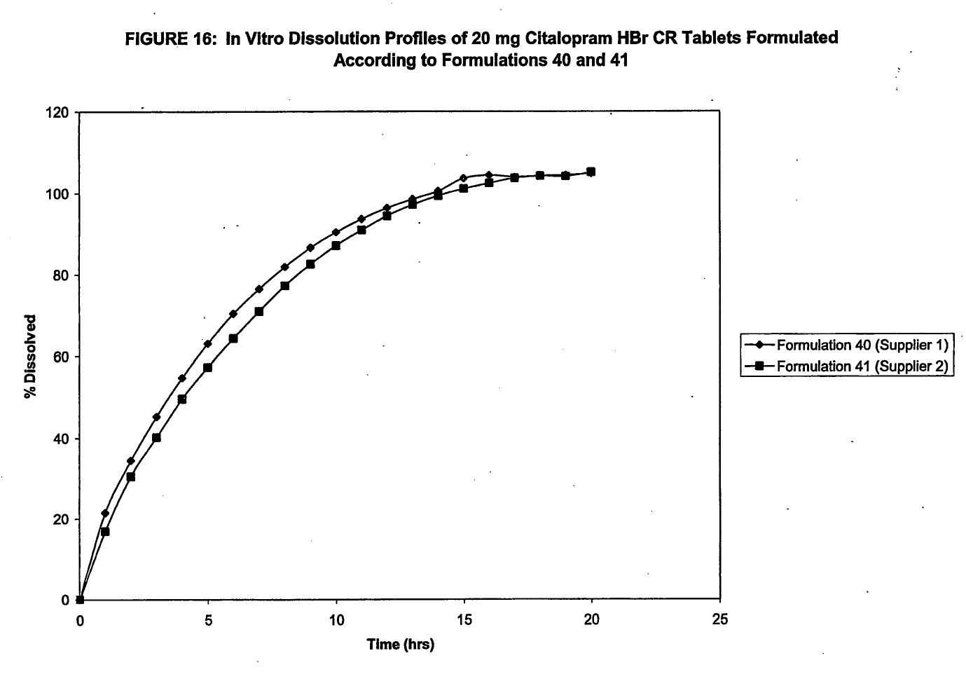

FIG. 16 compares the in vitro dissolution profiles of 20 mg Citalopram HBr CR Tablets formulated according to Formulations 40 and 41.

### Example 11

Particle size measurements were conducted on Paroxetine HCl. Table 40 provides the particle size measurement data of Paroxetine HCl.

TABLE 40
Particle Size Measurement Data of Paroxetine HCl
System Details

| | | |
|---|---|---|
| Dispersed in air | | |
| 1%-12% Obscuration, 1 000 sweeps | | |
| Range Lens: 100 mm | Beam Length: 10.00 mm | Sampler: MS64 |
| Obscuration: 6.9% | | |
| Presentation: 2RHA | [Particle R.I = (1.4500, 0.1000); | Dispersant R.I. = 1.000)] |
| Analysis Model: Polydisperse | | Residual: |
| Modifications: None | | |

Result Statistics

| | | |
|---|---|---|
| Distribution Type: Volume | Concentration = 0.0005% Vol | Uniformity = 1.980E+00 |
| D (v. 0.1) = 0.85 □mMedian, | D (v, 0.5) = 4.44 □m | D (v, 0.9) = 16.56 □m |
| Mean, D [4, 3] = 11.02 □m | Span = 3.541E+00 | |

Table 41 provides the composition of one Paroxetine HCl CR Tablet formulation (Formulation 42).

TABLE 41
Paroxetine HCl CR Tablet Formulation

| | Formulation 42 |
|---|---|
| Ingredients | (% w/w) |
| Paroxetine HCl | 5.6 |
| HPMC (K4MPrem CR) | 26.43 |
| Lactose Anhydrous | 57.14 |
| MCC | 8.05 |
| PVP (90F) | 1.76 |
| Magnesium Stearate | 1.02 |

In vitro dissolution studies were conducted on 20 mg Paroxetine HCl CR Tablets formulated according to Formulation 42. Table 42 provides the in vitro dissolution data of 20 mg Paroxetine HCl CR Tablets formulated according to Formulation 42.

TABLE 42
In vitro Dissolution Data of 20 mg Paroxetine HCl CR Tablets
Formulated According to Formulation 42

| | Formulation 42 |
|---|---|
| Time (Hrs) | (% dissolved) |
| 0 | 0 |
| 1 | 12.8 |
| 2 | 22.59 |
| 3 | 31.59 |
| 4 | 39.47 |
| 5 | 46.8 |
| 6 | 53.6 |
| 7 | 59.9 |
| 8 | 65.66 |
| 9 | 71.12 |
| 10 | 76.17 |
| 11 | 80.97 |
| 12 | 85.58 |
| 13 | 90.08 |
| 14 | 94.38 |
| 15 | 98.56 |
| 16 | 102.49 |

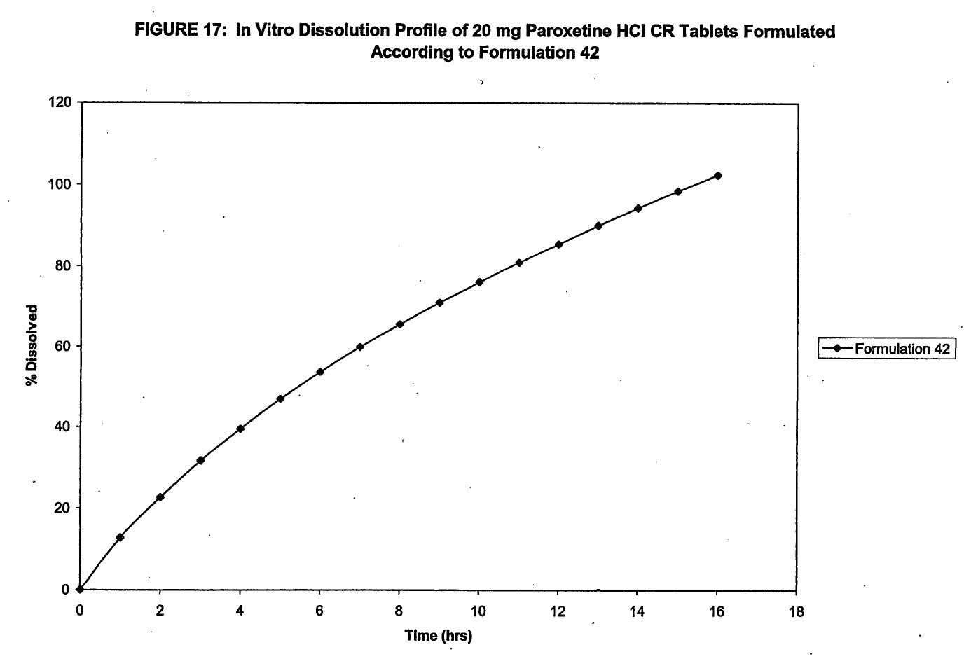

FIG. 17 provides the in vitro dissolution profile of 20 mg Paroxetine HCl CR Tablets formulated according to Formulation 42.

A randomized, two-way, single-does, open-label, cross-over pilot study was conducted to compare the rate and extent of absorption of the test product, 20 mg Paroxetine HCl CR Tablets versus the reference product, 20 mg PAXIL® Tablets (GlaxoSmithKline) under fasting conditions. The two study periods were separated by a three-week washout period. Twenty-four (24) blood samples were drawn for drug content analysis at 0.0 (pre-drug), 1.0, 2.0, 3.0, 4.0, 4.5, 5.0, 5.5, 6.0, 6.5, 7.0, 8.0, 10.0, 12.0, 14.0, 16.0, 24.0, 36.0, 48.0, 60.0, 72.0, 96.0, and 120.0 hours post-drug administration during each study period. Upon completion of the clinical portion of the study, all plasma samples were delivered to the analytical facility for the determination Paroxetine concentrations. Thirteen (13) qualified subjects were entered into the study. Pharmacokinetic and statistical analyses were conducted on the first twelve (12) evaluable subjects that completed the study.

Table 43 provides the mean plasma Paroxetine concentrations (ng/ml) over time after a single dose of one 20 mg Paroxetine HCl CR Tablet formulated according to Formulation 42 or after a single dose of one 20 mg PAXIL® tablet under fasting conditions.

TABLE 43
Mean Plasma Paroxetine Concentrations (n = 12)

| | Formulation 42 | PAXIL ® Tablet |
|---|---|---|
| Time | 1 × 20 mg | 1 × 20 mg |
| (hours) | (ng/ml) | (ng/ml) |
| 1 | 1.79 | 1.27 |
| 2 | 4.47 | 3.03 |
| 3 | 7.56 | 5.86 |
| 4 | 10.50 | 8.68 |
| 4.5 | 10.75 | 9.51 |
| 5 | 14.19 | 12.28 |
| 5.5 | 13.61 | 14.06 |
| 6 | 15.37 | 14.00 |
| 6.5 | 14.93 | 15.21 |
| 7 | 14.33 | 13.78 |
| 8 | 13.92 | 13.53 |
| 10 | 11.87 | 11.75 |
| 12 | 10.29 | 9.33 |
| 14 | 9.65 | 8.96 |
| 16 | 8.53 | 8.14 |
| 24 | 7.14 | 6.33 |
| 36 | 3.98 | 4.22 |
| 48 | 2.84 | 2.52 |
| 60 | 2.09 | 2.08 |
| 72 | 1.69 | 1.57 |
| 96 | 1.29 | 1.08 |
| 120 | 0.89 | 0.93 |

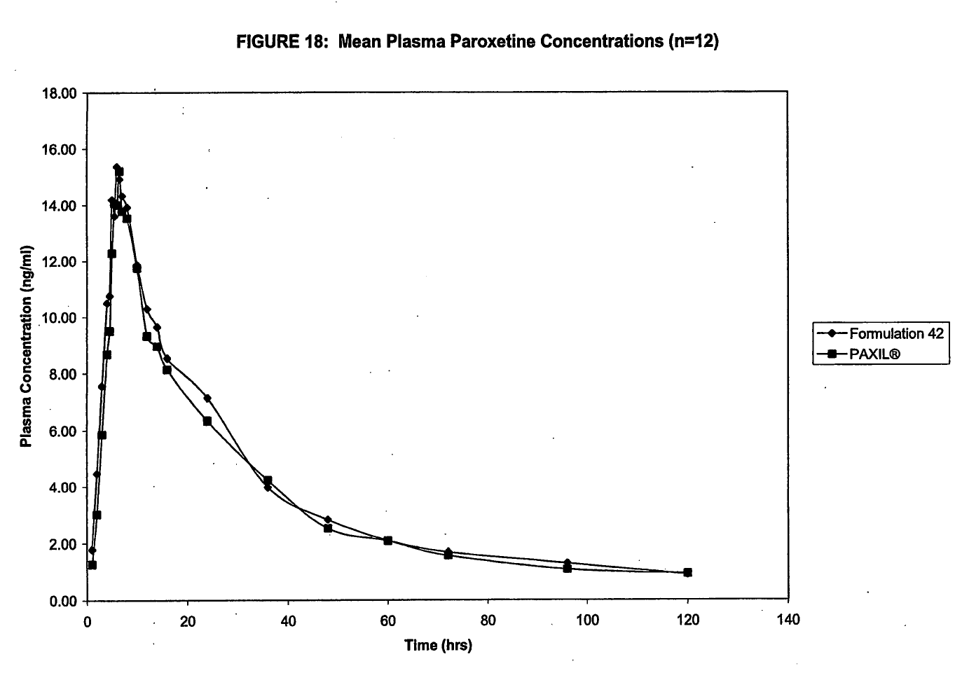

FIG. 18 illustrates the mean plasma Paroxetine concentrations (ng/ml) over time after a single-dose of one 20 mg Paroxetine HCl CR Tablet formulated according to Formulation 42 or after a single dose of one 20 mg PAXIL® tablet under fasting conditions.

Table 44 provides the mean pharmacokinetic parameters for plasma Paroxetine after a single dose of one 20 mg Paroxetine HCl CR Tablet formulated according to Formulation 42 or after a single dose of one 20 mg PAXIL® tablet under fasting conditions.

TABLE 44
Mean Pharmacokinetic Parameters for Plasma Paroxetine (n = 12)

| | | | | | | | Formulation | |
|---|---|---|---|---|---|---|---|---|
| | | | | | | | 42/ | |
| | Formulation 42 | | | PAXIL ® Tablet | | | PAXIL ® | |
| | 1 × 20 mg | | | 1 × 20 mg | | | Ratio | |
| | AUC | Cₘₐₓ | Tₘₐₓ | AUC | Cₘₐₓ | Tₘₐₓ | AUC | Cₘₐₓ |
| Mean | 440.50 | 16.61 | 6.13 | 421.60 | 16.39 | 6.58 | 1.08 | 1.11 |
| Std. Dev. | 567.89 | 10.52 | 1.23 | 540.40 | 11.50 | 1.33 | 0.27 | 0.29 |
| CV (%) | 128.92 | 63.31 | 20.03 | 128.18 | 70.17 | 20.18 | 25.24 | 25.88 |
| GeoMean | 249.14 | 13.44 | 6.01 | 237.52 | 12.48 | 6.48 | 1.05 | 1.08 |

Formulation 42/PAXIL ®

| | | | 90% |
|---|---|---|---|
| | Arith.Mean | GeoMean | C.I. |
| AUC | 1.04 | 1.05 | 91-121 |
| Cₘₐₓ | 1.01 | 1.08 | 94-123 |

Based on the 20 mg Paroxetine HCl CR Tablet formulated according to Formulation 42 to the 20 mg PAXIL® Tablet comparison of plasma data for Paroxetine for the twelve (12) evaluable subjects, the 90% geometric confidence intervals for AUC and Cₘₐₓ were found to be within the 80%-125% range. Therefore the 20 mg Paroxetine HCl CR Tablet formulated according to Formulation 42 are bioequivalent to the 20 mg PAXIL® Tablet.

### Example 12

Fluoxetine HCl was provided by two different suppliers (Supplier 1 and Supplier 2). Particle size measurements were conducted on Fluoxetine HCl provided by Supplier 1 and Supplier 2. Tables 45A and 45B provide the particle size measurement data of Fluoxetine HCl provided by Supplier 1 and Supplier 2, respectively.

TABLE 45A
Particle Size Measurement Data of Fluoxetine HCl Provided by
Supplier 1
Condition

| | | | | | |
|---|---|---|---|---|---|
| T % | 97.0% | Dist. Form: | Std. | Sampling times: | 1 [symbol] |
| (He—Ne): | | | | | |
| Trigger: | NO | R.R. Index: | 1.64-0.00i | | |
| Shaker: | 170 | Air P.: | LOW | Vacuum: | [symbol] |

Format

| | | | | | |
|---|---|---|---|---|---|
| Dist. Base: | Volume | Scalling: | Auto | Axis: | LogX − Linear Y |

Data

| | | | | | |
|---|---|---|---|---|---|
| Median: | 19.672□m | SP. Area: | 3861 cm²/cm² | S.D.: | 73.374□m |
| Mode: | 18.600□m | Mean: | 45.505□m | | |
| C.V.: | 161.24% | | | | |

Span: (D 20.0-D 10.0)/D50 = 0.158

| | | | | | |
|---|---|---|---|---|---|
| Dia.on % | (90.0%): | 118.199□m | % on Dia. | (2.000□m): | 0. [symbol] |
| Dia.on % | (70.0%): | 29.4885□m | % on Dia. | (10.000□m): | 16. [symbol] |
| Dia.on % | (35.0%): | 15.114□m | % on Dia. | (20.000□m): | 51. [symbol] |
| Dia.on % | (20.0%): | 10.941□m | % on Dia. | (100.000□m): | 88. [symbol] |
| Dia.on % | (10.0%): | 7.877□m | % on Dia. | (550.000□m): | 99. [symbol] |

[symbol]

indicates data missing or illegible when filed

TABLE 45B
Particle Size Measurement Data of Fluoxetine HCl Provided by
Supplier 2
Condition

| | | | | | |
|---|---|---|---|---|---|
| T % | 97.2% | Dist. Form: | Std. | Sampling times: | 1 [symbol] |
| (He—Ne): | | | | | |
| Trigger: | NO | R.R. Index: | 1.64-0.00i | | |
| Shaker: | 170 | Air P.: | HIGH | Vacuum: | [symbol] |

Format

| | | | | | |
|---|---|---|---|---|---|
| Dist. Base: | Volume | Scaling: | Auto | Axis: | LogX − Linear Y |

Data

| | | | | | |
|---|---|---|---|---|---|
| Median: | 20.638□m | SP. Area: | 5004 cm²/cm² | S.D.: | 124.996□m |
| Mode: | 18.612□m | Mean: | 86.383□m | | |
| C.V.: | 144.70% | | | | |

Span: (D 20.0-D 10.0)/D50 = 0.178

| | | | | | |
|---|---|---|---|---|---|
| Dia.on % | (90.0%): | 296.388□m | % on Dia. | (2.000□m): | 2. [symbol] |
| Dia.on % | (70.0%): | 42.5575□m | % on Dia. | (10.000□m): | 21. [symbol] |
| Dia.on % | (35.0%): | 14.546□m | % on Dia. | (20.000□m): | 48. [symbol] |
| Dia.on % | (20.0%): | 9.337□m | % on Dia. | (100.000□m): | 74. [symbol] |
| Dia.on % | (10.0%): | 5.668□m | % on Dia. | (580.000□m): | 99. [symbol] |

[symbol]

indicates data missing or illegible when filed

Table 46 provides the composition of two Fluoxetine HCl CR Tablet formulations, wherein the Fluoxetine HCl is provided by two different suppliers [Formulation 43 (Supplier 1) and Formulation 44 (Supplier 2)].

TABLE 46
Fluoxetine HCl CR Tablet Formulations

| | Formulation 43 | Formulation 44 |
|---|---|---|
| | (Supplier 1) | (Supplier 2) |
| Ingredients | (% w/w) | (% w/w) |
| Fluoxetine | 5.64 | 5.64 |
| HPMC (K4M Prem CR) | 26.42 | 26.42 |
| Lactose Anhydrous | 57.12 | 57.12 |
| MCC | 8.05 | 8.05 |
| PVP | 1.76 | 1.76 |
| Magnesium Stearate | 1.01 | 1.01 |

In vitro dissolution studies were conducted on 40 mg Fluoxetine HCl CR Tablets formulated according to Formulations 43 and 44. Table 47 provides the in vitro dissolution data of 40 mg Fluoxetine HCl CR Tablets formulated according to Formulations 43 and 44.

TABLE 47
In vitro Dissolution Data of 20 mg Fluoxetine HCl CR Tablets
Formulated According to Formulations 43 and 44

| | Formulation 43 | Formulation 44 |
|---|---|---|
| | (Supplier 1) | (Supplier 2) |
| Time (hrs) | (% dissolved) | (% dissolved) |
| 0 | 0 | 0 |
| 1 | 16.44 | 15.32 |
| 2 | 28.35 | 27.36 |
| 3 | 38.4 | 37.37 |
| 4 | 46.85 | 45.93 |
| 5 | 54.35 | 53.38 |
| 6 | 61.12 | 59.99 |
| 7 | 67.14 | 65.92 |
| 8 | 72.49 | 71.23 |
| 9 | 77.44 | 76.11 |
| 10 | 82.01 | 80.65 |
| 11 | 86.31 | 84.83 |
| 12 | 90.37 | 88.71 |
| 13 | 94.07 | 92.21 |
| 14 | 97.36 | 95.19 |
| 15 | 99.97 | 97.8 |
| 16 | 102.01 | 99.89 |
| 17 | 103.64 | 101.35 |
| 18 | 104.96 | 102.77 |
| 19 | 106.42 | 103.97 |

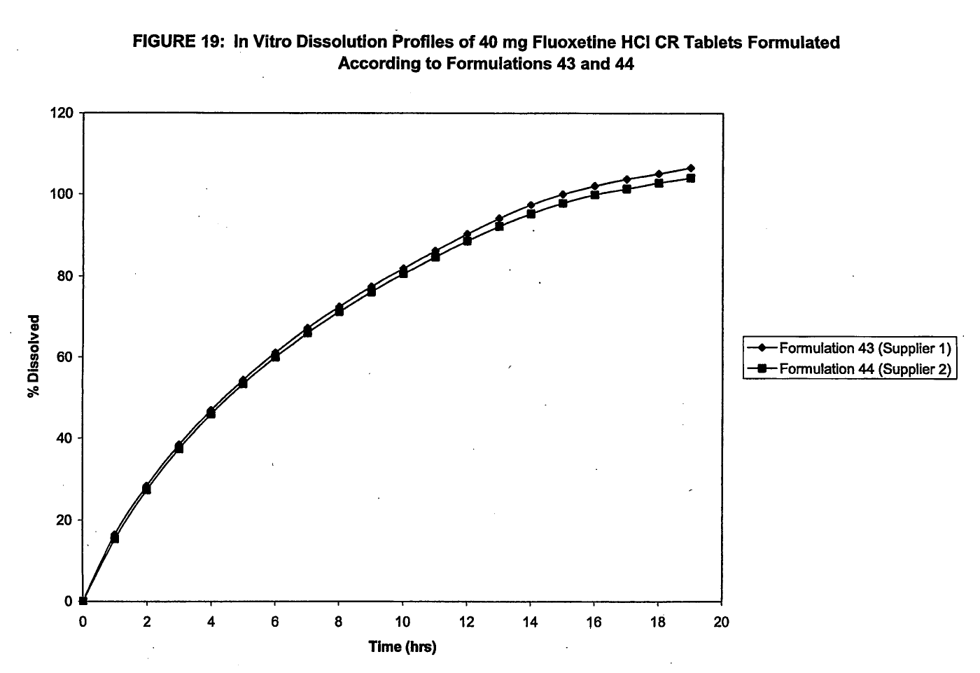

FIG. 19 compares the in vitro dissolution profiles of 40 mg Fluoxetine HCl CR Tablets formulated according to Formulations 43 and 44.

A randomized, two-way, single-dose, open-label, cross-over pilot study was conducted to compare the rate and extent of absorption of the test product, 40 mg Fluoxetine HCl CR Tablets versus the reference product, 40 mg PROZAC® PULVULES® (Eli Lilly and Company for Dista; Lot Number: 3MA11; Expiry Date: Dec. 1, 2001) under fasting conditions. The two study periods were separated by a three-week washout period. Twenty-four (24) blood samples were drawn for drug content analysis at 0.0 (pre-drug), 1.0, 2.0, 3.0, 4.0, 5.0, 6.0, 7.0, 8.0, 9.0, 10.0, 12.0, 14.0, 16.0, 24.0, 36.0, 48.0, 60.0, 72.0, 84.0, 96.0, 120.0, 168.0, and 288.0 hours post-drug administration during each study period. Upon completion of the clinical portion of the study, all plasma samples were delivered to the analytical facility for the determination of Fluoxetine and Norfluoxetine concentrations. Sixteen (16) qualified subjects were entered into the study. Pharmacokinetic and statistical analyses were conducted on first twelve (12) evaluable subjects that completed the study.

Table 48 provides the mean plasma Fluoxetine concentrations (ng/ml) over time after a single dose of one 40 mg Fluoxetine HCl CR Tablet formulated according to Formulation 43 or after a single dose of one 40 mg PROZAC® PULVULE® under fasting conditions.

TABLE 48
Mean Plasma Fluoxetine Concentrations (n = 12)

| | Formulation 43 | PROZAC ® PULVULE ® |
|---|---|---|
| | 1 × 40 mg | 1 × 40 mg |
| SAMPLE TIME | (ng/ml) | (ng/ml) |
| (hours) | MEAN VALUE ± SD | MEAN VALUE ± SD |
| 0.00 | 0.00 ± 0.00 | 0.00 ± 0.00 |
| 1.00 | 0.49 ± 0.35 | 0.96 ± 0.68 |
| 2.00 | 2.63 ± 1.78 | 6.06 ± 4.67 |
| 3.00 | 7.55 ± 4.07 | 12.20 ± 5.90 |
| 4.00 | 12.99 ± 7.20 | 18.12 ± 8.68 |
| 5.00 | 18.67 ± 8.57 | 23.20 ± 8.32 |
| 6.00 | 22.45 ± 8.55 | 26.43 ± 6.95 |
| 7.00 | 23.73 ± 7.22 | 27.11 ± 5.75 |
| 8.00 | 24.70 ± 7.83 | 28.49 ± 7.16 |
| 9.00 | 25.05 ± 7.35 | 29.62 ± 7.39 |
| 10.00 | 26.43 ± 8.09 | 26.70 ± 7.82 |
| 12.00 | 25.93 ± 6.90 | 25.79 ± 7.07 |
| 14.00 | 24.51 ± 7.21 | 25.87 ± 7.65 |
| 16.00 | 22.65 ± 7.60 | 23.22 ± 7.91 |
| 24.00 | 16.54 ± 5.73 | 17.44 ± 5.90 |
| 36.00 | 14.47 ± 5.88 | 15.24 ± 5.79 |
| 48.00 | 10.59 ± 4.79 | 11.28 ± 5.14 |
| 60.00 | 7.92 ± 4.59 | 8.68 ± 3.98 |
| 72.00 | 6.03 ± 3.21 | 7.14 ± 3.90 |
| 84.00 | 5.58 ± 3.26 | 5.57 ± 3.16 |
| 96.00 | 4.42 ± 2.70 | 4.66 ± 2.91 |
| 120.00 | 2.83 ± 2.14 | 3.01 ± 2.13 |
| 168.00 | 1.21 ± 1.21 | 1.27 ± 1.15 |
| 288.00 | 0.16 ± 0.32 | 0.17 ± 0.27 |

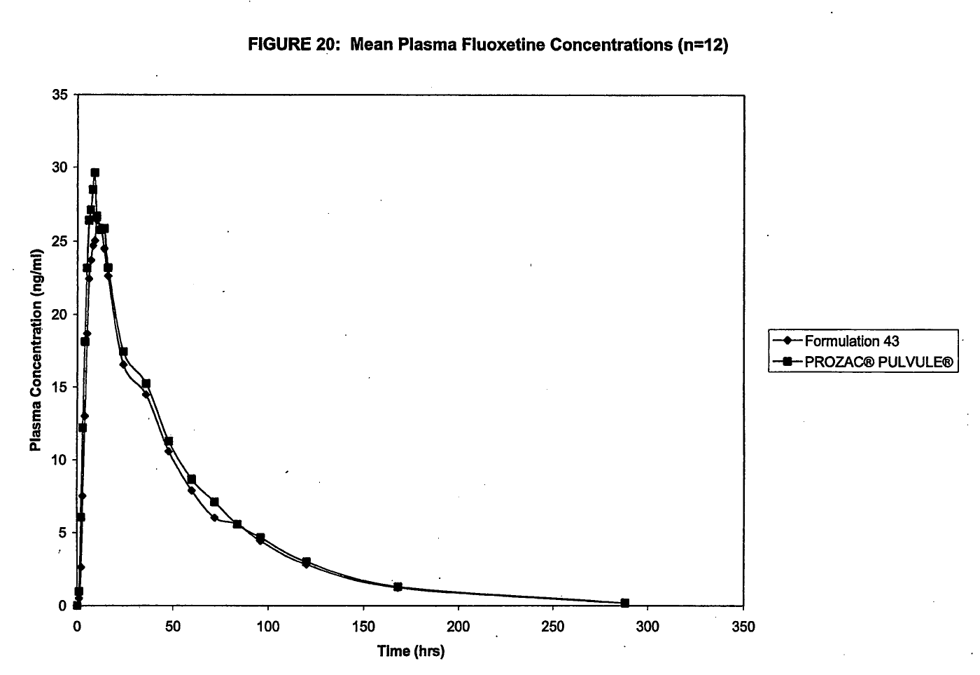

FIG. 20 illustrates the mean plasma Fluoxetine concentrations (ng/ml) over time after a single dose of one 40 mg Fluoxetine HCl CR Tablet formulated according to Formulation 43 or after a single dose of one 40 mg PROZAC® PULVULE® under fasting conditions.

Table 49 provides the mean pharmacokinetic parameters for plasma Fluoxetine after a single dose of one 40 mg Fluoxetine HCl CR Tablet formulated according to Formulation 43 or after a single dose of one 40 mg. PROZAC® PULVULE® under fasting conditions.

TABLE 49
Mean Pharmacokinetic Parameters for Plasma Fluoxetine (n = 12)

| | Formulation 43 | PROZAC ® PULVULE ® |
|---|---|---|
| | (Fasting) | (Fasting) |
| | 1 × 40 mg | 1 × 40 mg |
| Parameter | Mean ± SD | Mean ± SD |
| AUC₍₀₋ₜ₎ (ng · hr/ml) | 1385.34 ± 633.25 | 1475.84 ± 634.93 |
| AUC_{(0-inf)} (ng · hr/ml) | 1412.00 ± 647.76 | 1502.66 ± 638.92 |
| Cₘₐₓ (ng/ml) | 28.32 ± 7.84 | 30.55 ± 7.46 |
| Tₘₐₓ (hours) | 10.00 ± 2.30 | 8.00 ± 1.41 |
| t_{1/2} (hours) | 36.13 ± 9.00 | 35.91 ± 9.04 |
| Kₑₗ (hour⁻¹) | 00.20 ± 0.004 | 0.020 ± 0.005 |

Table 50 provides the comparison of plasma Fluoxetine data after a single dose of one 40 mg Fluoxetine HCl CR Tablet formulated according to Formulation 43 versus after a single dose of one 40 mg PROZAC® PULVULE® under fasting conditions.

TABLE 50
Comparison of Plasma Fluoxetine Data
(Formulation 43 vs. PROZAC ® PULVULE ®)

| | AUC₍₀₋ₜ₎ | AUC_{(0-inf)} | Cₘₐₓ |
|---|---|---|---|
| 90% Geometric C.I.¹ | 87%-100% | 87%-99% | 88%-95% |
| Ratio of Means² | 93% | 93% | 92% |
| CV³ | 9.14% | 8.73% | 5.18% |

¹90% Geometric Confidence Interval using log-transformed data and PROZAC ® PULVULES ® as the reference calculated based on two-way Formulation 43 vs. PROZAC ® PULVULES ® comparison.
²Calculated using geometric means according to the formula: e^{(Formulation 43 - PROZAC ® PULVULES ®)} × 100% calculated based on two-way Formulation 43 vs. PROZAC ® PULVULES ® comparison.
³Intra-subject coefficient of variation for log-transformed pharmacokinetic parameter, calculated based on two-way Formulation 43 vs. PROZAC ® PULVULES ® comparison.

Table 51 provides the mean plasma Norfluoxetine concentrations (ng/ml) over time after a single dose of one 40 mg Fluoxetine HCl CR Tablet formulated according to Formulation 43 or after a single dose of one 40 mg PROZAC® PULVULE® under fasting conditions.

TABLE 51
Mean Plasma Norfluoxetine Concentrations (n = 12)

| | Formulation 43 | PROZAC ® PULVULE ® |
|---|---|---|
| | (1 × 40 mg) | (1 × 40 mg) |
| SAMPLE TIME | (ng/ml) | (ng/ml) |
| (hours) | MEAN VALUE ± SD | MEAN VALUE ± SD |
| 0.00 | 1.85 ± 3.71 | 1.43 ± 1.78 |
| 1.00 | 1.92 ± 4.06 | 1.50 ± 1.61 |
| 2.00 | 2.04 ± 3.27 | 2.76 ± 1.80 |
| 3.00 | 3.36 ± 3.39 | 4.66 ± 2.33 |
| 4.00 | 5.02 ± 3.50 | 6.10 ± 2.66 |
| 5.00 | 7.39 ± 4.05 | 7.94 ± 3.16 |
| 6.00 | 8.79 ± 3.67 | 9.82 ± 3.44 |
| 7.00 | 10.77 ± 3.10 | 10.65 ± 4.01 |
| 8.00 | 11.56 ± 4.03 | 12.57 ± 4.53 |
| 9.00 | 11.84 ± 4.20 | 13.24 ± 4.25 |
| 10.00 | 13.53 ± 5.34 | 12.62 ± 4.05 |
| 12.00 | 13.93 ± 3.64 | 15.15 ± 4.85 |
| 14.00 | 15.41 ± 4.75 | 16.68 ± 5.05 |
| 16.00 | 16.02 ± 5.24 | 16.72 ± 5.40 |
| 24.00 | 14.98 ± 4.03 | 15.26 ± 4.53 |
| 36.00 | 19.77 ± 4.48 | 20.78 ± 4.58 |
| 48.00 | 19.91 ± 5.09 | 20.33 ± 5.22 |
| 60.00 | 19.58 ± 4.53 | 21.59 ± 4.98 |
| 72.00 | 21.03 ± 6.06 | 21.57 ± 5.11 |
| 84.00 | 21.53 ± 6.08 | 20.51 ± 3.80 |
| 96.00 | 20.12 ± 5.33 | 20.04 ± 4.93 |
| 120.00 | 18.91 ± 5.85 | 20.03 ± 6.16 |
| 168.00 | 16.08 ± 6.92 | 16.37 ± 5.16 |
| 288.00 | 10.23 ± 6.72 | 10.49 ± 4.80 |

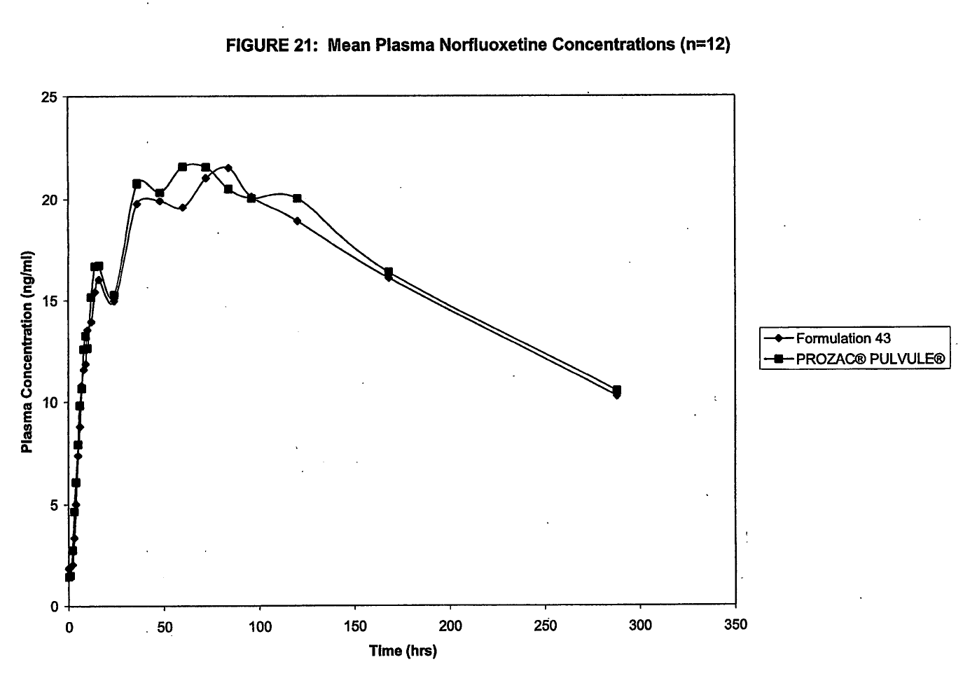

FIG. 21 illustrates the mean plasma Norfluoxetine concentrations (ng/ml) over time after a single dose of one 40 mg Fluoxetine HCl CR Tablet formulated according to Formulation 43 or after a single dose of one 40 mg PROZAC®PULVULE® formulation under fasting conditions.

Table 52 provides the mean pharmacokinetic parameters for plasma Norfluoxetine after a single dose of one 40 mg Fluoxetine HCl CR Tablet formulated according to Formulation 43 or after a single dose of one 40 mg PROZAC® PULVULE® under fasting conditions.

TABLE 52
Mean Pharmacokinetic Parameters for Plasma Norfluoxetine (n = 12)

| | Formulation 43 | PROZAC ® PULVULE ® |
|---|---|---|
| | (Fasting) | (Fasting) |
| | 1 × 40 mg | 1 × 40 mg |
| Parameter | Mean ± SD | Mean ± SD |
| AUC₍₀₋ₜ₎ (ng · hr/ml) | 4673.09 ± 1549.55 | 4586.11 ± 1122.06 |
| AUC_{(0-inf)} (ng · hr/ml) | 7843.69 ± 3837.59 | 6619.57 ± 2390.69 |
| Cₘₐₓ (ng/ml) | 23.91 ± 5.79 | 25.01 ± 5.06 |
| Tₘₐₓ (hours) | 84.06 ± 19.90 | 84.04 ± 39.30 |
| t_{1/2} (hours) | 189.63 ± 87.67 | 172.25 ± 62.06 |
| Kₑₗ (hour⁻¹) | 0.005 ± 0.003 | 0.005 ± 0.002 |

Table 53 provides the comparison of plasma Norfluoxetine data after a single dose of one 40 mg Fluoxetine HCl CR Tablet formulated according to Formulation 43 versus after a single dose of one 40 mg PROZAC® PULVULE® under fasting conditions.

TABLE 53
Comparison of Plasma Fluoxetine Data
(Formulation 43 vs. PROZAC ® PULVULE ®)

| | AUC₍₀₋ₜ₎ | AUC_{(0-inf)} | Cₘₐₓ |
|---|---|---|---|
| 90% Geometric C.I.¹ | 89%-112% | 77%-101% | 87%-103% |
| Ratio of Means² | 100% | 88% | 95% |
| CV³ | 16.09% | 8.75% | 11.47% |

¹90% Geometric Confidence Interval using log-transformed data and PROZAC ® PULVULES ® as the reference calculated based on two-way Formulation 43 vs. PROZAC ® PULVULES ® comparison.
²Calculated using geometric means according to the formula: e^{(Formulation 43 - PROZAC ® PULVULES ®)} × 100% calculated based on two-way Formulation 43 vs. PROZAC ® PULVULES ® comparison.
³Intra-subject coefficient of variation for log-transformed pharmacokinetic parameter, calculated based on two-way Formulation 43 vs. PROZAC ® PULVULES ® comparison.

Based on the 40 mg plasma data for Fluoxetine for the twelve (12) evaluable subjects, the 40 mg Fluoxetine HCl CR Tablet formulated according to Formulation 43 to the 40 mg PROZAC® PULVULE®, the 90% geometric confidence intervals for AUC₍₀₋ₜ₎, AUC_{(0-inf)}, and Cₘₐₓ were found to be within the 80%-125% range.

Based on the 40 mg Fluoxetine HCl CR Tablet formulated according to Formulation 43 to the 40 mg PROZAC® PULVULE® comparison of plasma data for Norfluoxetine for the twelve (12) evaluable subjects, the 90% geometric confidence intervals for AUC₍₀₋ₜ₎ and Cₘₐₓ were found to be within the 80%-125% range. Therefore, the 40 mg Fluoxetine HCl CR Tablets formulated according to Formulation 43 are bioequivalent to the 40 mg PROZAC® PULVULES®.

A randomized pilot, two-way, single dose, open-label, cross over study was conducted to compare the rate and extent of absorption of the test product, 40 mg Fluoxetine HCl CR Tablets versus the reference product, 40 mg PROZAC® PULVULES® (By: Eli Lilly and Company For: Dista; Lot Number: 3MA11M; Expiry Date: Dec. 1, 2001) under fasting conditions. The two study periods were separated by a three-week washout period. Twenty-four (24) blood samples were drawn for drug content analysis on Day 1 at 0.0 (pre-drug), 1.0, 2.0, 3.0, 4.0, 5.0, 6.0, 7.0, 8.0, 9.0, 10.0, 12.0, 14.0 and 16.0 hours post-drug administration; on Day 2 at 24.0 and 36.0 hours post-drug administration; on Day 3 at 48.0 and 60.0 hours post-drug administration; on Day 4 at 72.0 and 84.0 hours post-drug administration; on Day 5 at 96.0 hours post-drug administration; on Day 6 at 120.0 hours post-drug administration; on Day 8 at 168.0 hours post-drug administration; and on Day 13 at 288.0 hours post-drug administration. Upon completion of the clinical portion of the study, all plasma samples were delivered to the analytical facility for the determination the concentrations of Fluoxetine and its metabolite, Norfluoxetine. Eleven (11) qualified subjects and four (4) alternates were entered into the study. Pharmacokinetic and statistical analyses were conducted on twelve (12) evaluable subjects that completed the study.

Table 54 provides the mean plasma Fluoxetine concentrations (ng/ml) over time after a single dose of one 40 mg Fluoxetine HCl CR Tablet formulated according to Formulation 43 or after a single dose of one 40 mg PROZAC® PULVULE® under fasting conditions.

TABLE 54
Mean Plasma Fluoxetine Concentrations (n = 12)

| | Formulation 43 | PROZAC ® PULVULE ® |
|---|---|---|
| | (1 × 40 mg) | (1 × 40 mg) |
| SAMPLE TIME | (ng/ml) | (ng/ml) |
| (hours) | MEAN VALUE ± SD | MEAN VALUE ± SD |
| 0.00 | 0.00 ± 0.00 | 0.00 ± 0.00 |
| 1.00 | 1.06 ± 0.80 | 1.28 ± 1.06 |
| 2.00 | 3.88 ± 1.98 | 4.68 ± 3.00 |
| 3.00 | 10.01 ± 5.38 | 10.89 ± 4.96 |
| 4.00 | 14.06 ± 5.87 | 14.63 ± 5.93 |
| 5.00 | 17.79 ± 5.78 | 18.11 ± 7.77 |
| 6.00 | 21.42 ± 4.70 | 18.94 ± 7.48 |
| 7.00 | 22.83 ± 4.92 | 20.00 ± 7.52 |
| 8.00 | 22.99 ± 5.11 | 23.16 ± 5.74 |
| 9.00 | 23.75 ± 5.36 | 21.84 ± 4.14 |
| 10.00 | 21.76 ± 3.95 | 21.26 ± 4.14 |
| 12.00 | 21.76 ± 4.96 | 22.01 ± 5.50 |
| 14.00 | 19.41 ± 5.13 | 19.85 ± 4.52 |
| 16.00 | 17.06 ± 3.43 | 17.03 ± 4.70 |
| 24.00 | 13.13 ± 2.99 | 12.87 ± 3.66 |
| 36.00 | 11.28 ± 3.33 | 11.25 ± 3.38 |
| 48.00 | 7.61 ± 2.28 | 7.85 ± 2.27 |
| 60.00 | 6.44 ± 2.40 | 6.10 ± 1.90 |
| 72.00 | 4.68 ± 2.06 | 4.28 ± 1.61 |
| 84.00 | 3.86 ± 1.60 | 3.57 ± 1.33 |
| 96.00 | 2.68 ± 1.13 | 2.32 ± 0.97 |
| 120.00 | 1.66 ± 0.74 | 1.56 ± 0.66 |
| 168.00 | 0.62 ± 0.35 | 0.45 ± 0.35 |
| 288.00 | 0.00 ± 0.00 | 0.01 ± 0.05 |

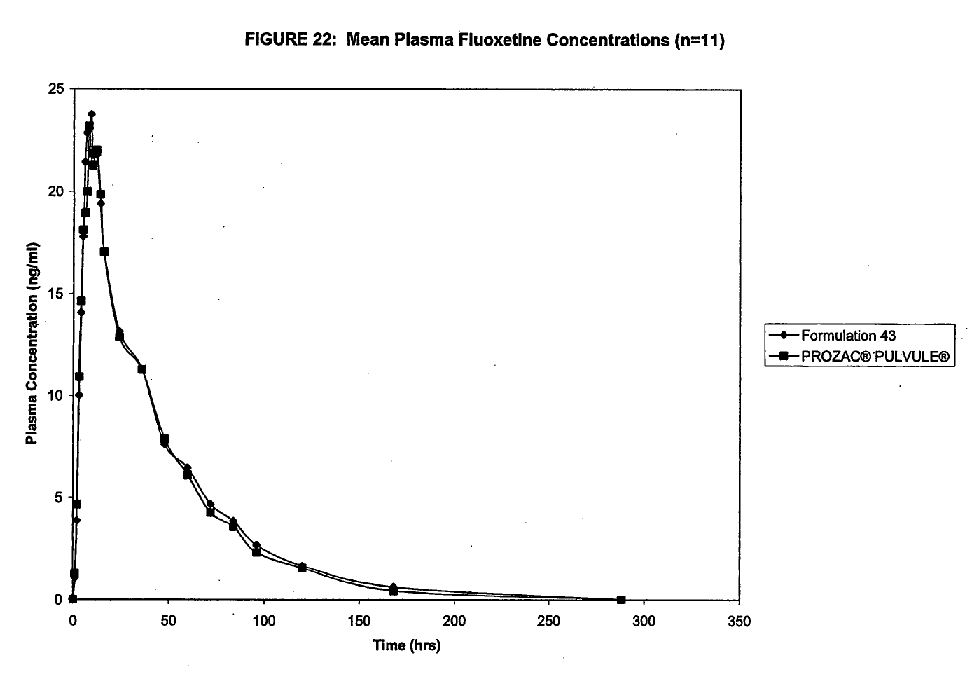

FIG. 22 illustrates the mean plasma Fluoxetine concentrations (ng/ml) over time after a single dose of one 40 mg Fluoxetine HCl CR Tablet formulated according to Formulation 43 or after a single dose of one 40 mg PROZAC®PULVULE®.

Table 55 provides the mean pharmacokinetic parameters for plasma Fluoxetine after a single dose of one 40 mg Fluoxetine HCl CR Tablet formulated according to Formulation 43 or after a single dose of one 40 mg PROZAC® PULVULE® under fasting conditions.

TABLE 55
Mean Pharmacokinetic Parameters for Plasma Fluoxetine (n = 12)

| | Formulation 43 | PROZAC ® PULVULE ® |
|---|---|---|
| | (Fasting) | (Fasting) |
| | 1 × 40 mg | 1 × 40 mg |
| Parameter | Mean ± SD | Mean ± SD |
| AUC₍₀₋ₜ₎ (ng · hr/ml | 1000.20 ± 277.33 | 981.17 ± 240.34 |
| AUC_{(0-inf)} (ng · hr/ml) | 1031.25 ± 289.90 | 1008.24 ± 245.60 |
| Cₘₐₓ (ng/ml) | 25.86 ± 5.90 | 24.42 ± 5.39 |
| Tₘₐₓ (hours) | 8.17 ± 1.53 | 8.58 ± 2.35 |
| t_{1/2} (hours) | 31.37 ± 4.87 | 31.74 ± 7.29 |
| Kₑₗ (hour⁻¹) | 0.023 ± 0.004 | 0.023 ± 0.004 |

Table 56 provides the comparison of plasma Fluoxetine data after a single dose of one 40 mg Fluoxetine HCl CR Tablet formulated according to Formulation 43 versus after a single dose of one 40 mg PROZAC® PULVULE® under fasting conditions.

TABLE 56
Comparison of Plasma Fluoxetine Data
(Formulation 43 vs. PROZAC ® PULVULE ®)

| | AUC₍₀₋ₜ₎ | AUC_{(0-inf)} | Cₘₐₓ |
|---|---|---|---|
| 90% Geometric C.I.¹ | 96%-106% | 96%-106% | 100%-112% |
| Ratio of Means² | 101% | 101% | 106% |
| CV³ | 6.88% | 6.79% | 7.70% |

¹90% Geometric Confidence Interval using log-transformed data and PROZAC ® PULVULES ® as the reference calculated based on two-way Formulation 43 vs. PROZAC ® PULVULES ® comparison.
²Calculated using geometric means according to the formula: e^{(Formulation 43 - PROZAC ® PULVULES ®)} × 100% calculated based on two-way Formulation 43 vs. PROZAC ® PULVULES ® comparison.
³Intra-subject coefficient of variation for log-transformed pharmacokinetic parameter, calculated based on two-way Formulation 43 vs. PROZAC ® PULVULES ® comparison.

Table 57 provides the mean plasma Norfluoxetine concentrations
(ng/ml) over time after a single dose of one 40 mg Fluoxetine HCl CR Tablet formulated according to Formulation 43 or after a single dose of one 40 mg PROZAC® PULVULE® under fasting conditions.

TABLE 57
Mean Plasma Norfluoxetine Concentrations (n = 12)

| | Formulation 43 | PROZAC ® PULVULE ® |
|---|---|---|
| | (1 × 40 mg) | (1 × 40 mg) |
| SAMPLE TIME | (ng/ml) | (ng/ml) |
| (hours) | MEAN VALUE ± SD | MEAN VALUE ± SD |
| 0.00 | 0.78 ± 1.29 | 0.72 ± 0.91 |
| 1.00 | 1.05 ± 1.35 | 0.84 ± 0.88 |
| 2.00 | 2.13 ± 1.66 | 1.84 ± 0.96 |
| 3.00 | 3.60 ± 1.88 | 3.51 ± 1.30 |
| 4.00 | 4.92 ± 2.09 | 5.05 ± 1.27 |
| 5.00 | 6.83 ± 2.38 | 6.71 ± 2.76 |
| 6.00 | 8.78 ± 2.43 | 8.25 ± 3.15 |
| 7.00 | 9.74 ± 2.78 | 9.87 ± 3.65 |
| 8.00 | 11.37 ± 2.48 | 11.49 ± 2.12 |
| 9.00 | 12.25 ± 2.62 | 12.42 ± 2.26 |
| 10.00 | 12.52 ± 2.78 | 12.38 ± 2.08 |
| 12.00 | 13.76 ± 3.16 | 15.72 ± 5.15 |
| 14.00 | 13.76 ± 3.39 | 14.38 ± 3.08 |
| 16.00 | 13.81 ± 3.15 | 14.57 ± 3.61 |
| 24.00 | 13.14 ± 2.74 | 14.71 ± 4.19 |
| 36.00 | 19.26 ± 4.33 | 20.81 ± 5.12 |
| 48.00 | 16.46 ± 2.67 | 18.56 ± 2.84 |
| 60.00 | 18.79 ± 3.84 | 20.59 ± 6.84 |
| 72.00 | 17.12 ± 4.10 | 16.50 ± 2.39 |
| 84.00 | 17.90 ± 2.93 | 19.43 ± 2.94 |
| 96.00 | 15.85 ± 3.31 | 15.13 ± 3.83 |
| 120.00 | 14.65 ± 3.07 | 14.84 ± 4.35 |
| 168.00 | 12.81 ± 3.56 | 11.50 ± 3.15 |
| 288.00 | 6.17 ± 2.25 | 7.23 ± 3.56 |

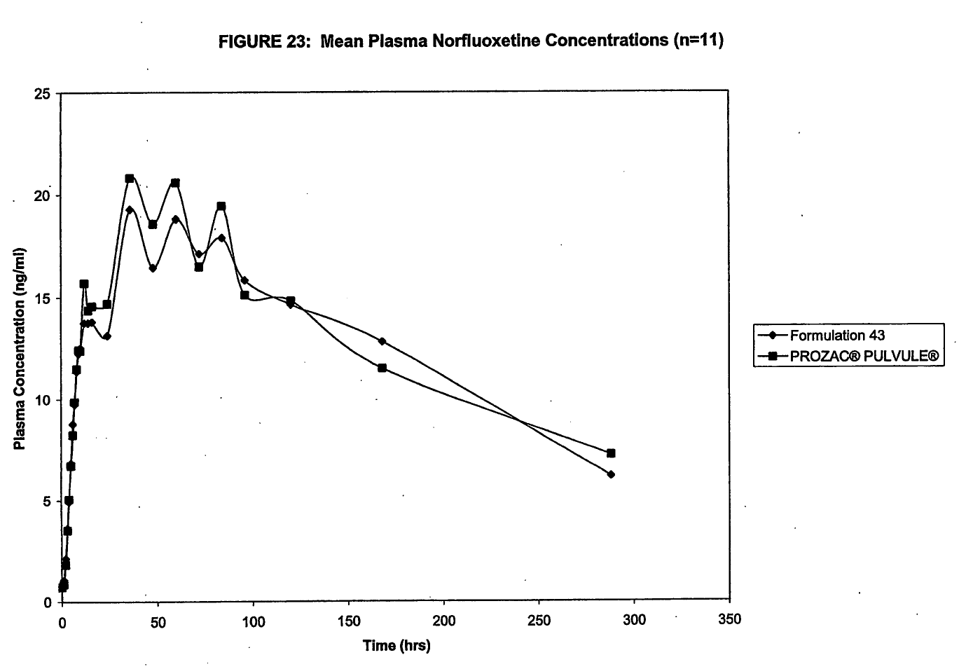

FIG. 23 illustrates the mean plasma Norfluoxetine concentrations (ng/ml) over time after a single dose of one 40 mg Fluoxetine HCl CR Tablet formulated according to Formulation 43 or after a single dose of one 40 mg PROZAC® PULVULE® under fasting conditions.

Table 58 provides the mean pharmacokinetic parameters for plasma Norfluoxetine after a single dose of one 40 mg Fluoxetine HCl CR Tablet formulated according to Formulation 43 or after a single dose of one 40 mg PROZAC® PULVULE® under fasting conditions.

TABLE 58
Mean Pharmacokinetic Parameters for Plasma Norfluoxetine (n = 12)

| | Formulation 43 | PROZAC ® PULVULE ® |
|---|---|---|
| | (Fasting) 1 × 40 mg | (Fasting) 1 × 40 mg |
| Parameter | Mean ± SD | Mean ± SD |
| AUC₍₀₋ₜ₎ (ng · hr/ml) | 3493.60 ± 730.93 | 3752.49 ± 871.15 |
| AUC_{(0-inf)} (ng · hr/ml) | 5190.67 ± 1584.60 | 5176.36 ± 1622.25 |
| Cₘₐₓ (ng/ml) | 20.67 ± 3.55 | 22.63 ± 5.53 |
| Tₘₐₓ (hours) | 54.01 ± 20.79 | 59.14 ± 20.86 |
| t_{1/2} (hours) | 148.84 ± 48.99 | 152.29 ± 60.35 |
| Kₑₗ (hour⁻¹) | 0.005 ± 0.002 | 0.005 ± 0.001 |

Table 59 provides the comparison of plasma Norfluoxetine data after a single dose of one 40 mg Fluoxetine HCl CR Tablets formulated accordin gto Formulation 43 versus after a single dose of one 40 mg PROZAC® PULVULE® under fasting conditions.

TABLE 59
Comparison of Plasma Norfluoxetine Data
(Formulation 43 vs. PROZAC ® PULVULE ®)

| | AUC₍₀₋ₜ₎ | AUC_{(0-inf)} | Cₘₐₓ |
|---|---|---|---|
| 90% Geometric C.I.¹ | 84%-103% | 92%-104% | 86%-99% |
| Ratio of Means² | 93% | 98% | 92% |
| CV³ | 13.87% | 7.36% | 9.28% |

¹90% Geometric Confidence Interval using log-transformed data and PROZAC ® PULVULES ® as the reference calculated based on two-way Formulation 43 vs. PROZAC ® PULVULES ® comparison.
²Calculated using geometric means according to the formula: e^{(Formulation 43 - PROZAC ® PULVULES ®)} × 100% calculated based on two-way Formulation 43 vs. PROZAC ® PULVULES ® comparison.
³Intra-subject coefficient of variation for log-transformed pharmacokinetic parameter, calculated based on two-way Formulation 43 vs. PROZAC ® PULVULES ® comparison.

Based on the 40 mg Fluoxetine HCl CR Tablet formulated according to Formulation 43 to the 40 mg PROZAC® PULVULE® comparison of plasma data for Fluoxetine and its metabolite Norfluoxetine, the 90% geometric confidence intervals for AUC₍₀₋ₜ₎, AUC_{(0-nfP} and Cₘₐₓ were found to be within the 80%-125% range. The results presented herein demonstrate that the test product, 40 mg Fluoxetine HCl CR Tablets formulated according to Formulation 43 is bioequivalent to the reference product, 40 mg PROZAC® PULVULES® (By: Eli Lilly and Company For Dista; Lot Number: 3MA11M; Expiry ate: Dec. 1, 2001) under fasting conditions.

A randomized, pilot study was conducted to compare the rate and extent of absorption of the test product, 40 mg Fluoxetine HCl CR Tablets to the reference product, 40 mg PROZAC® PULVULES® (Eli Lilly and Company for Dista), under fasting conditions. The two (2) study periods were separated by a three (3)-week washout period. Twenty-four (24) blood samples were drawn for drug content analysis at 0.0 (pre-drug), 1.0, 2.0, 3.0, 4.0, 5.0, 6.0, 7.0, 8.0, 9.0, 10.0, 12.0, 14.0, 16.0, 24.0, 36.0, 48.0, 60.0, 72.0, 84.0, 96.0, 120.0, 168.0 and 288.0 hours post-drug administration during each study period. Upon completion of the clinical portion of the study, all plasma samples were delivered to the analytical facility for the determination of Fluoxetine and Norfluoxetine concentrations. Twelve (12) qualified subjects were entered into the study. Pharmaceutical and statistical analyses were conducted on eleven (11) evaluable subjects that completed the study.

Table 60 provides the mean plasma Fluoxetine concentrations (ng/ml) over time after a single dose of one 40 mg Fluoxetine HCl CR Tablet formulated according to Formulation 43 or after a single dose of one 40 mg PROZAC® PULVULES® formulation under fasting conditions.

TABLE 60
Mean Plasma Fluoxetine Concentrations (n = 11)

| | Formulation 43 | PROZAC ® PULVULE ® |
|---|---|---|
| | (1 × 40 mg) | (1 × 40 mg) |
| SAMPLE TIME | (ng/ml) | (ng/ml) |
| (hours) | MEAN VALUE ± SD | MEAN VALUE ± SD |
| 0.00 | 0.09 ± 0.29 | 0.00 ± 0.00 |
| 1.00 | 0.62 ± 0.49 | 1.46 ± 0.83 |
| 2.00 | 3.50 ± 2.49 | 6.92 ± 2.83 |
| 3.00 | 11.65 ± 6.83 | 15.77 ± 5.88 |
| 4.00 | 15.48 ± 6.69 | 23.71 ± 6.05 |
| 5.00 | 22.31 ± 8.73 | 25.13 ± 6.56 |
| 6.00 | 25.41 ± 6.31 | 27.06 ± 5.64 |
| 7.00 | 27.06 ± 6.32 | 28.08 ± 4.46 |
| 8.00 | 25.16 ± 6.51 | 28.02 ± 4.46 |
| 9.00 | 26.25 ± 7.08 | 28.09 ± 5.13 |
| 10.00 | 25.97 ± 6.24 | 26.70 ± 4.36 |
| 12.00 | 25.87 ± 6.61 | 27.53 ± 6.22 |
| 14.00 | 23.29 ± 5.59 | 24.32 ± 6.00 |
| 16.00 | 21.45 ± 5.95 | 21.83 ± 6.09 |
| 24.00 | 17.39 ± 5.62 | 17.37 ± 6.15 |
| 36.00 | 14.49 ± 6.40 | 16.05 ± 6.40 |
| 48.00 | 10.24 ± 5.18 | 10.60 ± 6.25 |
| 60.00 | 9.11 ± 5.45 | 9.11 ± 5.99 |
| 72.00 | 6.37 ± 4.50 | 6.28 ± 4.58 |
| 84.00 | 5.79 ± 4.69 | 6.24 ± 5.02 |
| 96.00 | 4.73 ± 4.00 | 4.37 ± 3.92 |
| 120.00 | 2.95 ± 3.10 | 3.23 ± 3.20 |
| 168.00 | 1.75 ± 2.80 | 1.66 ± 2.19 |
| 288.00 | 0.48 ± 1.21 | 0.45 ± 1.07 |

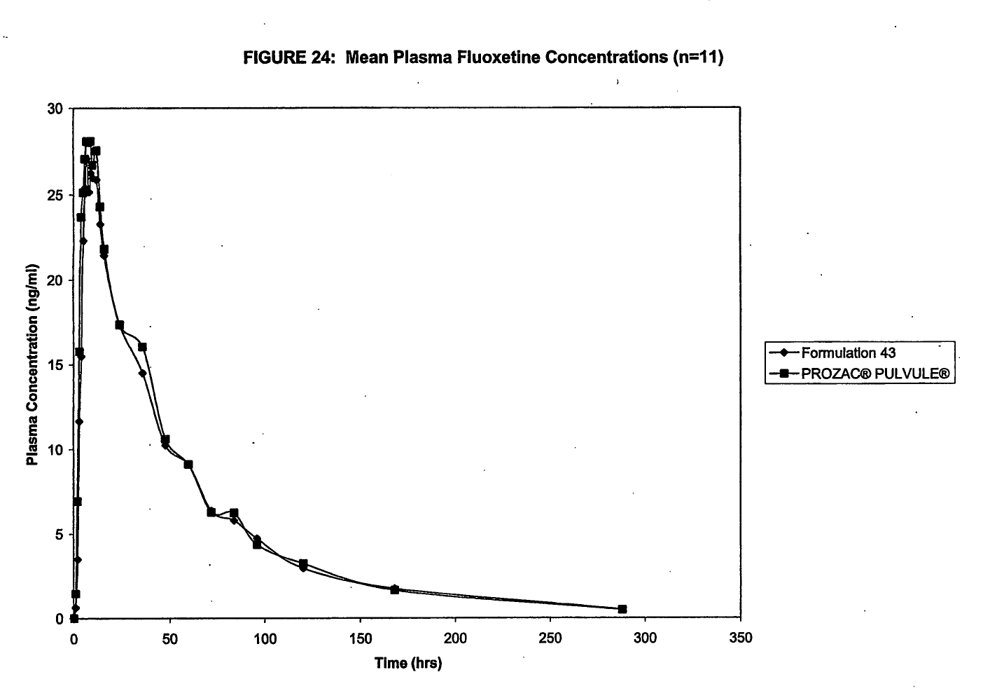

FIG. 24 illustrates the means plasma Fluoxetine concentrations (ng/ml) over time after a single dose of one 40 mg Fluoxetine HCl CR Tablet formulated according to Formulation 43 or after a single dose of one 40 mg PROZAC®PULVULE® under fasting conditions.

Table 61 provides the mean pharmacokinetic parameters for plasma Fluoxetine after a single dose of one 40 mg Fluoxetine HCl CR Tablet formulated according to Formulation 43 or after a single dose of one 40 mg PROZAC® PULVULE® formulation under fasting conditions.

TABLE 61
Mean Pharmacokinetic Parameters for Plasma Fluoxetine (n = 11)

| | Formulation 43 | PROZAC ® PULVULE ® |
|---|---|---|
| | (Fasting) 1 × 40 mg | (Fasting) 1 × 40 mg |
| Parameter | Mean ± SD | Mean ± SD |
| AUC₍₀₋ₜ₎ (ng · hr/ml | 1459.68 ± 880.20 | 1478.01 ± 863.53 |
| AUC_{(0-inf)} (ng · hr/ml) | 1534.26 ± 1033.14 | 1560.25 ± 987.47 |
| Cₘₐₓ (ng/ml) | 29.04 ± 6.39 | 31.39 ± 4.86 |
| Tₘₐₓ (hours) | 8.27 ± 2.72 | 7.27 ± 2.80 |
| t_{1/2} (hours) | 39.17 ± 23.85 | 40.59 ± 23.07 |
| Kₑₗ (hour⁻¹) | 0.021 ± 0.007 | 0.020 ± 0.007 |

Table 62 provides the comparison of plasma Fluoxetine data after a single dose of one 40 mg Fluoxetine HCl CR Tablet formulated according to Formulation 43 versus after a single dose of one 40 mg PROZAC® PULVULE® under fasting conditions.

TABLE 62
Comparison of Plasma Fluoxetine Data
(Formulation 43 vs. PROZAC ® PULVULE ®)

| | AUC₍₀₋ₜ₎ | AUC_{(0-inf)} | Cₘₐₓ |
|---|---|---|---|
| 90% Geometric C.I.¹ | 89%-106% | 89%-105% | 83%-100% |
| Ratio of Means² | 97% | 97% | 91% |
| CV³ | 10.96% | 10.72% | 11.87% |

¹90% Geometric Confidence Interval using log-transformed data and PROZAC ® PULVULES ® as the reference calculated based on two-way Formulation 43 vs. PROZAC ® PULVULES ® comparison.
²Calculated using geometric means according to the formula: e^{(Formulation 43 - PROZAC ® PULVULES ®)} × 100% calculated based on two-way Formulation 43 vs. PROZAC ® PULVULES ® comparison.
³Intra-subject coefficient of variation for log-transformed pharmacokinetic parameter, calculated based on two-way Formulation 43 vs. PROZAC ® PULVULES ® comparison.

Table 63 provides the mean plasma Norfluoxetine concentrations (ng/ml) over time after a single dose of one 40 mg Fluoxetine HCl CR Tablet formulated according to Formulation 43 or after a single dose of one 40 mg PROZAC® PULVULE® under fasting conditions.

TABLE 63
Mean Plasma Norfluoxetine Concentrations (n = 11)

| | Formulation 43 | PROZAC ® PULVULE ® |
|---|---|---|
| | (1 × 40 mg) | (1 × 40 mg) |
| SAMPLE TIME | (ng/ml) | (ng/ml) |
| (hours) | MEAN VALUE ± SD | MEAN VALUE ± SD |
| 0.00 | 0.87 ± 1.04 | 1.86 ± 2.75 |
| 1.00 | 1.11 ± 1.18 | 2.03 ± 2.68 |
| 2.00 | 1.84 ± 1.48 | 3.60 ± 2.61 |
| 3.00 | 4.27 ± 2.10 | 6.12 ± 3.17 |
| 4.00 | 5.73 ± 2.76 | 8.78 ± 4.23 |
| 5.00 | 8.14 ± 3.53 | 11.08 ± 4.19 |
| 6.00 | 10.47 ± 4.90 | 12.55 ± 4.87 |
| 7.00 | 11.78 ± 4.85 | 14.61 ± 5.18 |
| 8.00 | 13.12 ± 4.82 | 15.00 ± 4.94 |
| 9.00 | 12.63 ± 4.77 | 15.46 ± 5.64 |
| 10.00 | 14.56 ± 6.10 | 16.87 ± 5.58 |
| 12.00 | 16.19 ± 6.56 | 18.70 ± 6.24 |
| 14.00 | 16.26 ± 5.97 | 19.28 ± 7.13 |
| 16.00 | 16.74 ± 6.72 | 18.95 ± 6.72 |
| 24.00 | 17.67 ± 7.00 | 19.24 ± 7.49 |
| 36.00 | 21.15 ± 6.47 | 24.39 ± 8.58 |
| 48.00 | 20.80 ± 7.38 | 21.91 ± 7.53 |
| 60.00 | 19.85 ± 7.11 | 24.97 ± 8.18 |
| 72.00 | 20.85 ± 9.54 | 21.17 ± 6.34 |
| 84.00 | 22.72 ± 7.09 | 25.54 ± 8.65 |
| 96.00 | 22.17 ± 7.07 | 22.09 ± 6.46 |
| 120.00 | 20.29 ± 6.83 | 20.61 ± 7.73 |
| 168.00 | 16.04 ± 6.15 | 16.74 ± 6.69 |
| 288.00 | 9.55 ± 4.48 | 10.48 ± 5.18 |

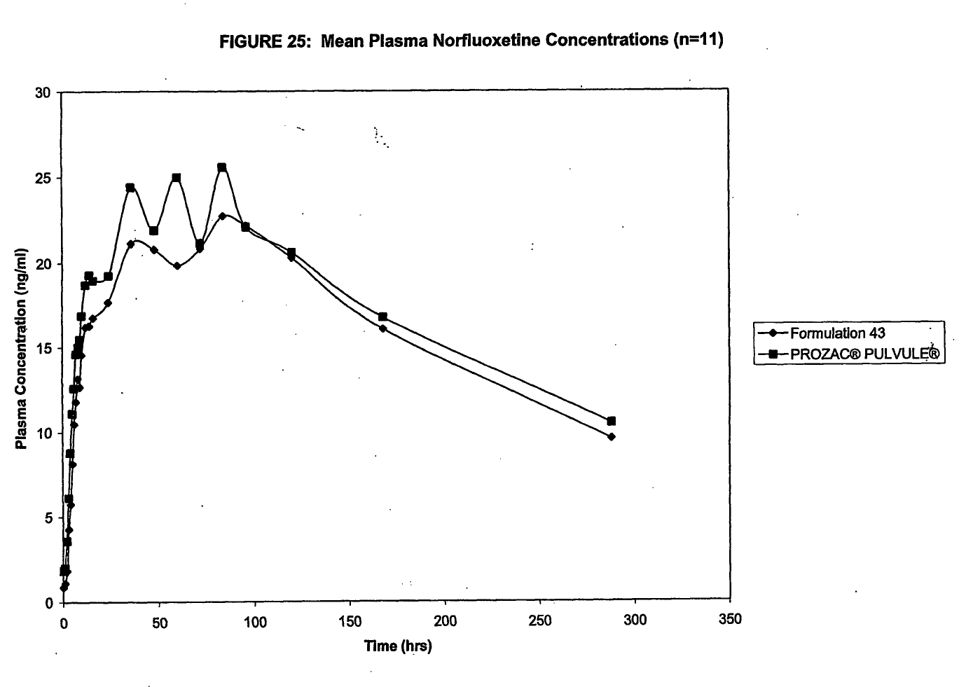

FIG. 25 illustrates the mean plasma Norfluoxetine concentrations (ng/ml) over time after a single dose of one 40 mg Fluoxetine HCl CR Tablet formulated according to Formulation 43 or after a single dose of one 40 mg PROZAC® PULVULE® under fasting conditions.

Table 64 provides the mean pharmacokinetic parameters for plasma Norfluoxetine after a single dose of one 40 mg Fluoxetine HCl CR Tablet formulated according to Formulation 43 or after a single dose of one 40 mg PROZAC® PULVULE® under fasting conditions.

TABLE 64
Mean Pharmacokinetic Parameters for Plasma Norfluoxetine (n = 11)

| | Formulation 43 | PULVULE ® PROZAC ® |
|---|---|---|
| | (Fasting) 1 × 40 mg | (Fasting) 1 × 40 mg |
| Parameter | Mean ± SD | Mean ± SD |
| AUC₍₀₋ₜ₎ (ng · hr/ml | 4606.15 ± 1582.52 | 5083.98 ± 1692.52 |
| AUC_{(0-inf)} (ng · hr/ml) | 7249.74 ± 2916.95 | 8348.86 ± 4502.44 |
| Cₘₐₓ (ng/ml) | 25.69 ± 6.88 | 27.36 ± 8.49 |
| Tₘₐₓ (hours) | 68.87 ± 20.83 | 64.19 ± 24.01 |
| t_{1/2} (hours) | 164.75 ± 64.02 | 183.18 ± 85.51 |
| Kₑₗ (hour⁻¹) | 0.005 ± 0.002 | 0.004 ± 0.001 |

Table 65 provides the comparison of plasma Norfluoxetine data after a Single dose of one 40 mg Fluoxetine HCl CR Tablet formulated according to Formulation 43 versus after a single dose, of one 40 mg PROZAC® PULVULE® under fasting conditions.

TABLE 65
Comparison of Plasma Norfluoxetine data
(Formulation 43 vs. PROZAC ® PULVULE ®)

| | AUC₍₀₋ₜ₎ | AUC_{(0-inf)} | Cₘₐₓ |
|---|---|---|---|
| 90% Geometric C.I.¹ | 82%-98% | 79%-102% | 91%-100% |
| Ratio of Means² | 90% | 90% | 95% |
| CV³ | 11.32% | 15.00% | 6.51% |

¹90% Geometric Confidence Interval using log-transformed data and PROZAC ® PULVULES ® as the reference calculated based on two-way Formulation 43 vs. PROZAC ® PULVULES ® comparison.
²Calculated using geometric means according to the formula: e^{(Formulation 43 - PROZAC ® PULVULES ®)} × 100% calculated based on two-way Formulation 43 vs. PROZAC ® PULVULES ® comparison.
³Intra-subject coefficient of variation for log-transformed pharmacokinetic parameter, calculated based on two-way Formulation 43 vs. PROZAC ® PULVULES ® comparison.

Based on the 40 mg Fluoxetine HCl CR Tablet formulated according to Formulation 43 to the 40 mg PROZAC® PULVULE® comparison of the plasma data for Fluoxetine, the 90% geometric confidence intervals for AUC₍₀₋ₜ₎, AUC_{(0-infinity)} and Cₘₐₓ were found to be within the 80%-125% range. Based on the comparison of the 40 mg Fluoxetine HCl CR Tablet formulated, according to Formulation 43 to the 40 mg PROZAC® PULVULE® comparison of plasma data for Norfluoxetine, the 90% geometric confidence intervals for AUC₍₀₋ₜ₎ and Cₘₐₓ were found to be within the 80%-125% range.

The results presented herein demonstrate that the test product, 40 mg Fluoxetine HCl CR Tablets formulated according to Formulation 43, is bioequivalent to the reference product, 40 mg PROZAC® PULVULES® (Eli Lily and Company for Dista; Lot Number: 3A11M; Expiry Date: Dec. 1, 2001) under fasting conditions.

Table 66 provides a list of the equipment used in the manufacturing process of SSRI CR Tablets formulated according to one embodiment of the present invention.

TABLE 66
Equipment Used in the Manufacturing Process of SSRI CR Tablets

| Process | Equipment |
|---|---|
| Wet granulation process | High shear mixer |
| Drying of wet mass | Tray dryer |
| Size reduction of dry granules | Cone mill |
| Preparation of bulk blend | V-blender |
| Tabletting process | Rotary Press |
| Coating process | Perforated coater |

The equipment used to manufacture the experimental batch and the pivotal biobatch formulated according to one embodiment of the present invention, although different in size, operate on the same principles as those used in the pilot batches (see Table 67). Commercial batches formulated to one embodiment of the present invention are manufactured using the same equipment used for the pivotal biobatch.

TABLE 67
Comparison of Critical Equipment Used in the Manufacturing
Process of SSRI CR Tablets

| Lab Scale Equipment | Scale up Equipment | Process Stage |
|---|---|---|
| High Shear Granulator | High Shear Granulator | Mixing and Granulation |
| Model: Aeromatic | Model: PMA 600 | |
| fielder | | |
| Tray Drying Oven | Tray Drying Oven | Drying the granules |
| Blue M electric | Model: O'Hara 400 | |
| Cone Mill | Cone Mill | Sizing of granules |
| Model: Model 197S | Model: KEK 220 | |
| V-Blender | V-Blender | Blending of granules |
| Model: 16 qt. | Model: 40 cu. ft. | |
| Rotary Tablet Press | Rotary Tablet Press | Manufacture of tablets |
| Model: Riva Pren | Model: Manesty | |
| | Unipress | |
| Polisher/De-duster | Polisher/De-duster | Removes loose particles |
| Model: FMC AV 100 | Model: MG2 GTS/E | from tablets |
| | Metal Detector | Rejects tablets with |
| | Model: LOMA | metal contaminant |
| Mixer | Propeller Mixer: | Preparation of Coating |
| Model: Craframo | ID# DTR-1/DTR-9 | Suspension |
| Mixer | Mixer | Mixing of coating |
| Model: Craframo | Model: Craframo | Solution |
| Tablet Coater | Tablet Coater | Coating of tablets |
| Model: Labcoat 11 | Model: O'Hara | |
| system | FastCoat60″ | |

The manufacture of SSRI CR Tablets, in one embodiment, is based on the wet granulation process. The manufacturing process can be divided into ten (10) unit operations as follows.
1. Weighing of active ingredients and excipients;
2. Blending of active ingredient and retardant excipient (polymer) in a high shear mixer;
3. Granulation of active ingredient and retardant excipient blend with alcoholic solution of Povidone in the high shear mixer;
4. Drying of wet granulation in a tray dryer;
5. Size reduction of dry granules in a cone mill;
6. Blending of dry granules with lubricants in a V-blender.
7. Tabletting of final blend in a Rotary press;
8. Preparation of coating solution;
9. Coating of tablets;
10. Packaging.

A brief description of various processes in the manufacture of SSRI CR Tablets formulated according to one embodiment of the present invention is as follows:
A) Wet granulation process: The weighed materials were transferred to the high shear mixer and materials were dry blended with samples removed for in-process quality control testing for potency in order to determine blend uniformity (homogeneity). Once blend uniformity was determined to be appropriate alcohol was added to the dry blend and granulated under high shear. The wet granules were discharged into trays and loaded into the tray dryer. The granules were dried for 10 hours at 60° C. Samples were removed for in-process quality control testing for residual of isopropyl alcohol and loss on drying. The tray dryer was unloaded based on whether specifications were met.
B) Preparation of bulk blend: The dried granules were passed through a 2 mm round screen in a cone mill for the purpose of size reduction. The size reduced granules were transferred into a V-blender. Magnesium stearate was added to the granules in the V-blender and blended. Samples were taken from the lubricated-blend for in-process quality control testing for potency determination and blending uniformity. The samples were also used to measure flowability, compressibility, granule size and distribution of the granules
C) Tabletting Process: The amount of granules required to give the potency of 20 mg active ingredient was calculated after which the hopper of a tablet press was charged. Compression force and fill weight were set by adjusting the relevant controls on the tablet-press. A few tablets were compressed by manual turning of the drive shaft. Physical specifications were checked and when ten tablets met the required specifications of weight, hardness, thickness, and friability automated tabletting was started.
D) Manufacture of Coating Suspension: The coating suspension was prepared by mixing OPADRY® II White in purified water to produce a suspension with the help of a propeller mixer. Samples were taken in order to measure total solids for quality control release testing.
E) Coating process: The amount of tablets to be coated were accurately weighed and charged into the coating pan. The tablets were then coated by spray coating with the coating suspension. Samples were taken to test for appearance, identification, potency, content uniformity, impurity levels, moisture content and dissolution for the purpose of quality control release testing.
F) Packaging process: The coated tablets are quarantined and released for packaging after quality control testing.

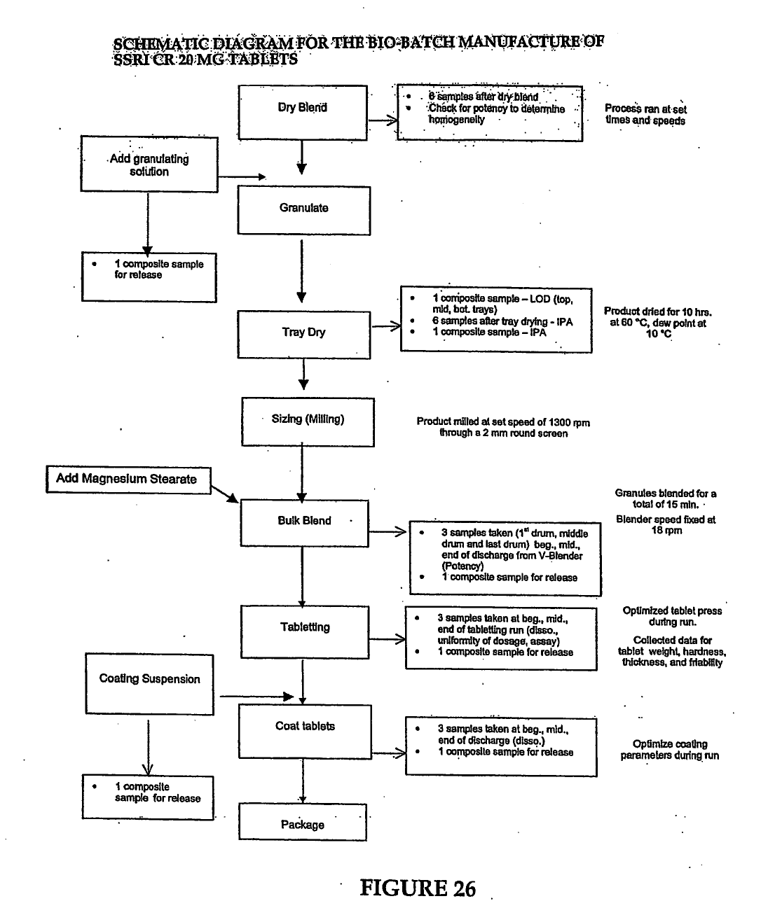

FIG. 26 illustrates a schematic diagram for the bio-batch manufacture of SSRI CR Tablets formulated according to one embodiment of the present invention.

The following parameters were identified as critical to successful formulation during the development of SSRI CR Tablets formulated according to one embodiment of the present invention, and in-process testing procedures were set to check these parameters during the relevant procedures. The tests were carried out and shown to conform to the set standards during manufacturing, as documented in the QA release testing data, before progressing to the next stage.
1. Homogeneity of blend during initial dry mixing: this was shown by content uniformity as evidenced from potency determination with low standard deviation;
2. Drying time and temperature: Adequate drying was: checked by determination of loss on drying and possible volatile impurities.
3. Milling and lubrication in V-blender: Potency determination showed homogeneity of blend in the V-blender, while flowability and compressibility of the blend as evidenced by Carr Index was an indicator of adequate lubrication. Particle size analysis of granules showed that the required size reduction was achieved by milling.
4. Tabletting: Tablets were within the specified limits for weight variability, hardness, thickness and friability. Assay showed that content uniformity and impurities all conformed to standards, while dissolution showed that the release rates were also within set limits.
5. Coating: The coated tablets were within specified limits for weight gain after the coating process. As expected, dissolution data of the coated tablets showed that the coating had no significant effect on the dissolution rate of the uncoated tablet.

Table 68 provides the in-process and finished product testing specifications established for the biobatches and proposed for the commercial batches of SSRI CR Tablets formulated according to one embodiment of the present invention.

TABLE 68
Quality Standard Data for 20 mg SSRI Coated CR Tablets

| Tests and methods | Specifications | Results |
|---|---|---|
| DESCRIPTION | White oblong coated tablet | White oblong |
| STM: 0006.00, Rev. 03 | | coated tablet |
| IDENTIFICATION | HPLC retention time of | HPLC retention |
| STM: 0022.27 | major peak in sample | time of major |
| | conforms with that of the | peak in sample |
| | Standard | conforms with |
| | | that of the |
| | | Standard |
| POTENCY | 90.0-110.0% | 101.0% |
| STM: 0022.07 | | |
| CONTENT UNIFORMITY | Min: 85.0% of LC | 102.7% |
| <0022.27> | _{Max}: 115.0% of LC | |
| | % RSD < 6.0% | 1.2% |
| DISSOLUTION | 1 Hour: 10-30% Range | 23% |
| STM: 0021.14 | 4 Hour: 46-66% Range | 54% |
| | 8 Hour: 70-90% Range | 80% |
| | 12 Hour: NLT 80% Range | 93% |
| IMPURITIES | [Lu 14-017]: NMT 0.1% | 0.0% |
| STM: 0022.13 | [Lu 29-215]: NMT 0.25% | 0.07% |
| | [Lu 11-305]: NMT: 0.1% | 0.02% |
| | [Lu 29-075]: NMT 0.25% | 0.07% |
| | Single Unknown Impurity: | None > 0.1% |
| | NMT 0.1% | |
| | Total Impurities: | 0.22% |
| | NMT 0.5% | |
| Moisture Content | NMT 5% | 1% |
| STM: 0009.01 | | |

While the foregoing provides a detailed description of a preferred embodiment of the invention, it is to be understood that this description is illustrative only of the principles of the invention and not limitative. Furthermore, as many changes can be made to the invention without departing from the scope of the invention, it is intended that all material contained herein be interpreted as illustrative of the invention and not in a limiting sense.

1. A modified release pharmaceutical composition for oral administration, suitable for once daily dosing, comprising
(a) from about 4.0% (w/w) to about 8.0% (w/w) of a form of at least one selective serotonin re-uptake inhibitor (SSRD selected from the group consisting of Fluoxetine, Fluvoxamine, Paroxetine, Sertraline, Venlafaxine, Citalopram, racemic mixtures thereof, enantiomers thereof, pharmaceutically acceptable salts thereof and combinations thereof;
(b) from about 10% (w/w) to about 40% (w/w) of at least one release rate controlling polymer:
(c) from about 40% (w/w) to about 60% (w/w) of at least one diluent;
(d) from about 0% (w/w) to about 5% (w/w) of at least one binder;
(e) from about 0% (w/w) to about 5.0% (w/w) of at least one lubricant;
(f) from about 0% (w/w) to about 6.0% (w/w) of at least one surfactant;
(g) from about 0% (w/w) to about 6.0% (w/w) of at least one solubilizing agent;
(h) from about 0% (w/w) to about 6.0% (w/w) of at least one bioavailability enhancer; and
(i) from about 0% (w/w) to about 6.0% (w/w) of at least one acidifying agent.

2. The pharmaceutical composition of claim 1 wherein the form of the at least one selective serotonin re-uptake inhibitor is selected from the group consisting of Fluoxetine HCl. Fluvoxamine maleate, Paroxetine HCl. Sertraline HCl. Venlafaxine HCl. Citalopram HBr, Escitalopram oxalate and combinations thereof.

3. The pharmaceutical composition of claim 1 or 2 wherein the at least one release rate controlling polymer is a solid sustained release pharmaceutically-acceptable polymer selected from the group consisting of at least one hydrophilic water-soluble polymer at least one hydrophobic water-insoluble polymer and combinations thereof.

4. The pharmaceutical composition of claim 3 wherein the at least one hydrophilic polymer is selected from the group consisting of cellulose derivatives, dextrans, starches, carbohydrates, base polymers, natural or hydrophilic gums, xanthans, alginates, gelatins, polyacrylic acids, polyvinyl alcohol, polyvinyl pyrrolidone, carbomers and combinations thereof.

5. The pharmaceutical composition of claim 4 wherein the cellulose derivatives are selected from the group consisting of cellulose ethers, cellulose esters and combinations thereof, and alkyl cellulose derivatives, hydroxyalkyl cellulose derivatives and combinations thereof.

6. The pharmaceutical composition of claim 5 wherein the alkyl and hydroxyalkyl cellulose derivatives are selected from the group consisting of methyl cellulose, ethylcellulose, hydroxylmethylcellulose, hydroxyl ethylcellulose, hydroxypropyl cellulose, hydroxypropyl methylcellulose, methylhydroxy ethylcellulose, methylhydroxy ethylcellulose, methylhydroxy propylcellulose, sodium carboxymethyl cellulose and combinations thereof.

7. The pharmaceutical composition of any one of claims 1 to 6 further comprising at least one other pharmaceutically acceptable excipient selected from the group consisting of at least one granulating aid, at least one colourant, at least one flavourant, at least one pH adjuster, at least one anti-adherent at least one glidant, at least one solubility enhancer, at least one surface active agent and combinations thereof.

8. The pharmaceutical composition of any preceding claim wherein the at least one diluent is selected from the group consisting of lactose, microcrystalline cellulose, mannitol and combinations thereof.

9. The pharmaceutical composition of any preceding claim wherein the at least one binder is polyvinyl pyrrolidone.

10. The pharmaceutical composition of any preceding claim wherein the at least one lubricant is selected from the group consisting of magnesium stearate, stearic acid and combinations thereof.

11. The pharmaceutical composition of any one of claims 1 to 10 wherein the at least one surfactant is selected from the group consisting of a bile salt, sodium lauryl sulphate (SLS), polyoxyethylene/polyoxypropylene block copolymers, polyethylene glycol hydrogenated castor oils, polyethylene glycols, saturated polyglycolized glycerides from hydrogenated vegetable oils, saturated polyglycolized glycerides, water soluble derivatives of natural source vitamins, sucrose stearate, mannitol, mono- and diglycerides, lauroyl macrogol glycerides, stearoyl macrogol glycerides, Vitamin E d-α-tocopheryl polyethylene glycol 1000 succinate propylene glycol monoesters of medium chain fatty acids and combinations thereof.

12. The pharmaceutical composition of any preceding claim wherein the at least one acidifying agent is L-tartaric acid.

13. The pharmaceutical composition of any preceding claim wherein the form of the at least one selective serotonin re-uptake inhibitor is incorporated in a matrix.

14. The pharmaceutical composition of claim 13 wherein the matrix is selected from the group consisting of a normal release matrix and a modified release matrix.

15. The pharmaceutical composition of claim 14 wherein the modified release matrix is selected from the group consisting of a sustained release matrix and a controlled release matrix.

16. The pharmaceutical composition of claim 14 or 15 further comprising a coating selected from the group consisting of a film coating and a modified release coating.

17. The pharmaceutical composition of claim 16 wherein the film coating is present in a concentration from about 0% (w/w) to about 5% (w/w) of the core.

18. The pharmaceutical composition of claim 16 wherein the modified release coating is selected from the group consisting of a delayed-release coating, an extended-release coating and combinations thereof.

19. The pharmaceutical composition of claim 18 wherein the modified release coating is an enteric coating.

20. The pharmaceutical composition of claim 18 wherein the extended release coating is selected from the group consisting of a sustained-release coating, a controlled-release coating, and combinations thereof.

21. The pharmaceutical composition of according to any one of claims 18 to 20 wherein the core is selected from the group consisting of a tablet, a spheroid, a bead, a microsphere, a seed, a pellet, and an ion-exchange resin bead.

22. The pharmaceutical composition of claim 20 wherein the pharmaceutical composition is a dosage form selected from the group consisting of a tablet, a capsule, a caplet, a sachet, and a troche.

23. The pharmaceutical composition of any one of claims 18-22 wherein the modified release coating is present in an amount of about 2% (w/w) to about 25% (w/w).

24. The pharmaceutical composition of claim 23 wherein the at least one hydrophobic polymer is selected from the group consisting of ethylcellulose, at least one acrylic polymer and combinations thereof.

25. The pharmaceutical composition of claim 24 wherein the at least one acrylic polymer is selected from the group consisting of acrylic acid copolymers, methacrylic acid copolymers, methyl methacrylate copolymers, ethoxyethyl methacrylates, cyanoethyl methacrylate, aminoalkyl methacrylate copolymer, poly (acrylic acid), poly (methacrylic acid, methacrylic acid alkylamide copolymer, poly (methyl methacrylate), poly (methacrylic acid) (anhydride), methyl methacrylate, polymethacrylate, methyl methacrylate copolymer, poly (methyl methacrylate), poly (methyl methacrylate) copolymer, polyacrylamide, aminoalkyl methacrylate copolymer, poly (methacrylic acid anhydride), glycidyl methacrylate copolymers and combinations thereof.

26. The pharmaceutical composition of claim 24 or 25 wherein the enteric coating comprises at least one methacrylic acid copolymer.

27. The pharmaceutical composition of claim 26 wherein the methacrylic acid copolymer is selected from the group consisting of methacrylic acid copolymer Type, A, methacrylic acid copolymer Type B, methacrylic acid copolymer Type C and combinations thereof.

28. The pharmaceutical composition of any one of claims 18-27 wherein the modified release film coating further comprises at least one plasticizer.

29. The pharmaceutical composition of claim 28 wherein the hydrophobic polymer is ethylcellulose and the at least one plasticizer is selected from the group consisting of dibutyl sebacate, diethyl phthalate triethyl citrate, tributyl citrate, triacetin, and combinations thereof.

30. The pharmaceutical composition of claim 28 or 29 wherein the at least one hydrophobic polymer is at least one acrylic polymer and the at least one plasticizer is selected from the group consisting of triethyl citrate, tributyl citrate, dibutyl phthalate, polyethylene glycol, propylene glycol, diethyl phthalate, castor oil, triacetin and combinations thereof.

31. The modified pharmaceutical composition of any preceding claim wherein the composition comprises:
(a) from about 4.0% (w/w) to about 8.0% (w/w) of the form of the at least one SSRI selected from the group consisting of Fluoxetine. Fluvoxamine, Paroxetine Sertraline, Venlafaxine, Citalopram, racemic mixtures thereof, enantiomers thereof, pharmaceutically acceptable salts thereof and combinations thereof;
(b) from about 10.0% (w/w) to about 40.0% (w/w) of hydroxypropyl methylcellulose 2208;
(c) from about 40.0% (w/w) to about 60.0% (w/w) of lactose anhydrous;
(d) from about 6.0% (w/w) to about 10.0% (w/w) of microcrystalline cellulose;
(e) from about 0.0% (w/w) to about 4.0% (w/w) of polyvinyl pyrrolidone;
(f) from about 0.0% (w/w) to about 2.0% (w/w) of magnesium stearate;
(g) from about 0.0% (w/w) to about 2.0% (w/w) of stearic acid;
(h) from about 0.0% (w/w) to about 2.0% (w/w) of a carbomer;
(i) from about 0.0% (w/w) to about 3.0% (w/w) of a bile salt;
(j) from about 0.0% (w/w) to about 3.0% (w/w) of sodium lauryl sulphate;
(k) from about 0.0% (w/w) to about 6.0% (w/w) of a poloxamer;
(l) from about 0.0% (w/w) to about 6.0% (w/w) of a polyethylene glycol hydrogenated castor oil;
(m) from about 0.0% (w/w) to about 6.0% (w/w) of a polyethylene glycol;
(n) from about 0.0% (w/w) to about 6.0% (w/w) of a saturated polyglycolized glyceride;
(o) from about 0.0% (w/w) to about 6.0% (w/w) of a water soluble derivative of a natural source vitamin;
(p) from about 0.0% (w/w) to about 6.0% (w/w) of sucrose stearate;
(q) from about 0.0% (w/w) to about 6.0% (w/w) of mannitol;
(r) from about 0.0% (w/w) to about 6.0% (w/w) of L-tartaric acid; and combinations thereof.

32. The pharmaceutical composition of any preceding claim wherein the form of the at least one SSRI is present in the pharmaceutical composition in an amount effective to treat at least one condition selected from the group consisting of depression, major depressive disorder, obsessive compulsive disorder, panic disorder, social anxiety disorder, generalized anxiety disorder, posttraumatic stress disorder and combinations thereof.

33. A modified release pharmaceutical composition comprising:

| | Ingredients | % (w/w) |
|---|---|---|
| (a) | Citalopram HBr | 4.0-8.0 |
| (b) | Hydroxypropyl methylcellulose | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinyl pyrrolidone (KOLLIDON ® K90F) | 0.5-3.5 |
| (f) | Magnesium stearate | 0.5-2.0 |

34. A modified release pharmaceutical composition comprising:

| | Ingredients | % (w/w) |
|---|---|---|
| (a) | Paroxetine HCl | 4.0-8.0 |
| (b) | Hydroxypropyl methylcellulose | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinyl Pyrrolidone (KOLLIDON ® K90F) | 0.5-3.5 |
| (f) | Magnesium stearate | 0.5-2.0 |

35. A modified release pharmaceutical composition comprising:

| | Ingredients | % (w/w) |
|---|---|---|
| (a) | Fluoxetine HCl | 4.0-8.0 |
| (b) | Hydroxypropyl methylcellulose | 10.0-40.0 |
| | (METHOCEL ® K4M Premium CR) | |
| (c) | Lactose Anhydrous (DT) | 40.0-60.0 |
| (d) | Microcrystalline Cellulose (AVICEL ® PH 101) | 6.0-10.0 |
| (e) | Polyvinyl pyrrolidone (KOLLINDON ® K90F) | 0.5-3.5 |
| (f) | Magnesium stearate | 0.5-2.0 |

36. The modified pharmaceutical composition of any one of claims 33 to 35 wherein the pharmaceutical composition is a matrix tablet.

37-214. (canceled)
